SEQUENCE LISTING (10 sequences)

SEQ ID NO: 1 - protein, 5 aa, Unknown
  Description of Unknown NFAT antagonist peptide motif sequence
VIVIT

SEQ ID NO: 2 - DNA, 62 bp, Artificial Sequence
  Description of Artificial Sequence Synthetic primer
gaattcatgtatccgtatgacgtaccagagtacgccatgtccgagcccaaggcgattgatcc

SEQ ID NO: 3 - DNA, 80 bp, Artificial Sequence
  Description of Artificial Sequence Synthetic primer
cgttaccgtcattatcgtcgttataggtcccgttcggctagggtttgggagaggaccctgagctgtcatgcactcgatcg

SEQ ID NO: 4 - DNA, 36 bp, Artificial Sequence
  Description of Artificial Sequence Synthetic primer
ctgccggcgtcgggccttccttcagtagtcttgatc

SEQ ID NO: 5 - DNA, 61 bp, Artificial Sequence
  Description of Artificial Sequence Synthetic primer
gaagttatcagtcgacatggactacaaagacgatgacgacaagggcaggaagtgtccacaa

SEQ ID NO: 6 - DNA, 58 bp, Artificial Sequence
  Description of Artificial Sequence Synthetic primer
ctttagtcacttaaactacaatgttttagatttttacagatcttcgaaagatctggta

SEQ ID NO: 7 - protein, 5 aa, Unknown
  Description of Unknown NFAT antagonist peptide motif sequence
VIVIT

SEQ ID NO: 8 - protein, 6 aa, Artificial Sequence
  Description of Artificial Sequence Synthetic consensus sequence
PXIXIT

SEQ ID NO: 9 - protein, 4 aa, Unknown
  Description of Unknown “KDEL” motif peptide
KDEL

SEQ ID NO: 10 - protein, 11 aa, Artificial Sequence
  Description of Artificial Sequence Synthetic peptide
RRRRRRRRRRR

(19) United States
(12) Patent Application Publication
(10) Pub. No.: US 2013/0195866 A1
(43) Pub. Date: Aug. 1, 2013

(54) METHODS TO INHIBIT NEURODEGENERATION

(71) Applicants: Brian J. Bacskai, Andover, MA (US); Bradley T. Hyman, Swampscott, MA (US); Kishore Kuchibhotla, Bethel, CT (US); Hai-Yan Wu, Roscoe, IL (US)

(72) Inventors: Brian J. Bacskai, Andover, MA (US); Bradley T. Hyman, Swampscott, MA (US); Kishore Kuchibhotla, Bethel, CT (US); Hai-Yan Wu, Roscoe, IL (US)

(21) Appl. No.: 13/574,006

(22) PCT Filed: Jan. 19, 2011
(86) PCT No.: PCT/US11/21625

§ 371 Date: Mar. 11, 2013

Related U.S. Application Data
(60) Provisional application No. 61/296,158, filed on Jan. 19, 2010.

(51) Int. Cl. A61K 38/08

(52) U.S. Cl. CPC A61K 38/08
USPC 424/135.1; 514/44 R; 514/17.8; 424/172.1; 435/375; 424/152.1; 424/133.1

(57) ABSTRACT

Disclosed herein are methods, and compositions for inhibiting neurodegeneration, e.g., in neuronal cells. The methods and compositions the invention can be used to treat a neurodegenerative disorder, e.g., Alzheimer's disease, Parkinson's disease, Huntington's disease, and frontotemproal dementia. In some embodiments, the methods and compositions can be used to inhibit neurodegeneration, e.g., caused by tau-mediated synaptic neurodegeneration, encephalitis, brain trauma, or any disorder suffering from weakening synapses.

### CROSS-REFERENCE TO RELATED APPLICATIONS

This International application paragraphs the benefit of priority under 35 U.S.C. §119(e) of U.S. Provisional Application Nos. 61/296,158, filed Jan. 19, 2010, the contents of each of which are incorporated herein by reference in their entirety.

### GOVERNMENT SUPPORT

This invention was made with government support under Grant No. AG08487, EB000768, and EY13399 awarded by National Institutes of Health (NIH). The government has certain rights in the invention.

### FIELD OF THE INVENTION

The invention relates generally to methods, and compositions for inhibiting neurodegeneration, and more particularly relates to methods, and compositions for treating a neurodegenerative disorder, e.g., Alzheimer's disease, Parkinson's disease, Huntington's disease and frontotemproal dementia. In some embodiments, the methods and compositions can be used to inhibit neurodegeneration, e.g., caused by tau-mediated synaptic neurodegeneration, encephalitis, brain trauma, or any disorder suffering from weakening synapses.

### BACKGROUND OF THE INVENTION

Alzheimer's disease (AD) is a devastating neurodegenerative disorder which is clinically characterized by deterioration of memory and cognitive function, progressive impairment of daily living activities, and several neuropsychiatric symptoms. Cummings, J. L., 351 N Engl J. Med. 56 (2004). Amyloid β (Aβ) peptide, the major component of senile plaques, accumulates in the brain of Alzheimer's disease (AD) patients (Walsh and Selkoe, 2004; Yaari and Corey-Bloom, 2007). The amyloid hypothesis (Hardy and Allsop, 1991; Hardy and Selkoe, 2002) suggests that Aβ induces neurodegeneration, but the key molecular mechanisms that link Aβ to neuronal damage remain a critical gap in understanding AD and designing effective therapeutics.

Exposure of neurons to synthetic or naturally secreted Aβ peptides induces a reduction of dendritic spines and synaptic dysfunction in culture and causes memory deficits in learned behavior in normal rats (Pike et al., 1993; Lorenzo and Yankner, 1994; Geula et al., 1998; Shankar et al., 2007, 2008). In vivo studies using longitudinal multiphoton microscopy reveal a direct toxic effect on neurites surrounding Aβ deposits, including dendritic simplification, loss of dendritic spines, and neuritic dystrophies (Spires et al., 2005; Meyer-Luehmann et al., 2008). However, the connection between extracellular Aβ and the intracellular signaling pathways that cause local disruption of neuronal processes, synaptic dysfunction, and neurodegeneration are unknown, thus hindering the development of an effective strategy for treatment of Alzheimer's disease.

Elevated intracellular calcium has been observed in neurons exposed to Aβ in several model systems, and disrupted calcium homeostasis has been suggested to play a central role in AD pathogenesis (Palotás et al., 2002; Mattson, 2004; Smith et al., 2005; Stutzmann, 2005; Bezprozvanny and Mattson, 2008; Busche et al., 2008). For example, Hyman B T et al. have previously shown that neurites in amyloid precursor protein (APP)-overexpressing transgenic mice have significantly elevated intracellular calcium ([Ca²⁺]ᵢ) compared with age-matched nontransgenic controls (Kuchibhotla et al., 2008). In neurons, Ca²⁺ signaling is tightly controlled to ensure proper functioning of numerous Ca²⁺-dependent events, including processes influenced by the serine/threonine phosphatase calcineurin (CaN) (Mulkey et al., 1994; Wang and Kelly, 1996; Halpain et al., 1998; Berridge et al., 2000).

CaN is the only Ca²⁺-activated protein phosphatase in neurons, and it is involved in many facets of neuronal physiology, including synaptic plasticity, and learning and memory (Klee et al., 1979; Winder and Sweatt, 2001). In mice, both genetic and pharmacological upregulation of the expression of CaN can induce synaptic dysfunction and memory impairment, whereas CaN inhibition strengthens memory in spatial learning tasks (Malleret et al., 2001; Winder and Sweatt, 2001; Mansuy, 2003). While CaN activity has been shown previously to play a role in AD pathogenesis, CaN as a therapeutic target for AD treatment may produce undesirable side effects because CaN participates in a number of cellular processes and Ca²⁺-dependent signal transduction pathways. Hence, in order to develop a targeted and effective approach for treatment of AD, it is imperative to uncover the downstream effectors of CaN involved in Alzheimer's disease.

AD is the leading cause of dementia in the elderly. As the incidence and prevalence of AD rise steadily with increasing longevity, AD threatens to become a catastrophic burden on health care, particularly in developed countries [Alzheimer's Disease Education & Referral Center: http://www.nia.nih.gov/Alzheimers/AlzheimersInformation/GeneralInfo]. However, there are very few therapeutic drugs or interventions effective for treatment of AD. As such, there is a strong need for developing a novel therapeutic strategy for treatment of AD.

### SUMMARY OF THE INVENTION

Aspects of the present invention stem from the discovery that even in the absence of amyloid precursor proteins (APPs) or amyloid-beta (Aβ), increased CaN-mediated NFAT activation is sufficient to produce similar phenotypes as Aβ-induced morphological deficits in neurons, e.g., dendritic spine loss, dendritic simplification, and neuritic dystrophies. It was also discovered that increased levels of an active form of CaN and NFATc4 are detected in the nuclear fraction from the cortex of patients with AD. Further, intracortical injection of a NFAT antagonist (e.g., a peptide comprising an amino acid sequence of VIVIT (SEQ ID NO: 7) to an in vivo model of AD inhibits plaque-associated neurodegenerative changes.

Accordingly, provided herein are methods, and compositions for inhibiting neurodegeneration. In one aspect, the method includes contacting a population of neuronal cells with an effective amount of a nuclear factor of activated T cells (NFAT) antagonist. The method described herein can be performed in vitro, ex vivo, or in vivo. In one embodiment, the method is performed in vivo in a subject, wherein the subject is diagnosed with or pre-disposed to a neurodegenerative disorder. In some embodiments, the neurodegenerative disorder is selected from the group consisting of: Alzheimer's disease, Parkinson's disease, Huntington's disease, frontotemproal dementia, encephalitis, brain trauma, tau-associated neurodegenerative disorder, amyloid-beta-associated neurodegenerative disorder, inflammation-associated neurodegenerative disorder, and any disorder suffering from weakening synapses.

In various embodiments, the NFAT antagonist can be selected from the group consisting of a small molecule, a nucleic acid, a protein, a peptide, and an intrabody. In one embodiment, the NFAT antagonist is a peptide comprising an amino acid sequence of VIVIT (SEQ ID NO: 7). In certain embodiments, the VIVIT-containing peptide can further include a sequence encoding a nuclear localization signal. In some embodiments, the NFAT peptide antagonist can be expressed by a vector, e.g., a viral vector.

In some embodiments, the effective amount of a NFAT antagonist is sufficient to decrease NFAT activity of one or more neuronal cells by at least about 5%, as compared to neuronal cells in the absence of the NFAT antagonist. In one embodiment, the NFAT antagonist decreases calcineurin-mediated NFAT activity. In some embodiments, the effective amount is sufficient to increase dendritic spine density of one or more neuronal cells by at least about 5%, as compared to neuronal cells in the absence of the NFAT antagonist. In other embodiments, the effective amount is sufficient to decrease neuritic dystrophies of one or more neural cells by at least about 5%, as compared to neuronal cells in the absence of the NFAT antagonist. In one embodiment, the effective amount of a NFAT antagonist is about 2 μM.

Neurodegeneration is involved in many neurodegenerative disorders, e.g., Alzheimer's disease. In certain embodiments, a NFAT antagonist can be introduced in vivo to a population of neuronal cells. The population of neuronal cells can be present in a subject, e.g., diagnosed with or pre-disposed to a neurodegenerative disorder such as Alzheimer's disease. Accordingly, another aspect of the invention provides a method of treating Alzheimer's disease (AD) in a subject in need thereof, the method comprising contacting a population of neuronal cells in the subject with an effective amount of a nuclear factor of activated T cells (NFAT) antagonist. In some embodiments, the method further comprises a step of diagnosing a subject with AD prior to the contacting. In various embodiments, the subject can be a mammal, e.g., a human.

In some embodiments, the population of neuronal cells in the subject can be contacted with a NFAT antagonist, e.g., by injection. The NFAT antagonist can be selected from the group consisting of a small molecule, a nucleic acid, a protein, and a peptide. For example, the NFAT antagonist can be a peptide comprising an amino acid sequence of VIVIT (SEQ ID NO: 7). In some embodiments, the VIVIT-containing peptide can further include a sequence encoding a nuclear localization signal. In other embodiments, the NFAT peptide antagonist can be expressed by a vector, e.g., a viral vector.

In some embodiments, the effective amount of a NFAT antagonist used in the method described herein is sufficient to decrease NFAT activity of one or more neuronal cells by at least about 5%, as compared to neuronal cells in the absence of the NFAT antagonist. In one embodiment, the NFAT antagonist decreases calcineurin-mediated NFAT activity. In some embodiments, the effective amount is sufficient to increase dendritic spine density of one or more neuronal cells by at least about 5%, as compared to neuronal cells in the absence of the NFAT antagonist. In other embodiments, the effective amount is sufficient to decrease neuritic dystrophies of one or more neural cells by at least about 5%, as compared to neuronal cells in the absence of the NFAT antagonist. In one embodiment, the effective, amount of a NFAT antagonist is about 10 mg/kg. In one embodiment, the effective amount of a NFAT antagonist is about 2 μM.

### BRIEF DESCRIPTION OF THE DRAWINGS

FIGS. 1A to 1E show abnormal morphologies in neurons from Tg cultures at 14 DIV. FIGS. 1A and 1B show representative images of a wild-type GFP-labeled neuron at 14 DIV with intricately branched dendritic arbors. FIG. 1B is a close-up image of the area outlined in FIG. 1A. FIGS. 1C and 1D show representative images of Tg neurons exhibiting simplified dendritic complexity and localized dendritic dystrophies. FIG. 1D is a close-up image of the area outlined in FIG. 1C. FIG. 1E shows that the percentage of neurons with dendritic dystrophies at 14 and 21 DIV was increased in Tg neurons compared with wild type (14 DIV: wild-type cultures, 2.0±0.2%; Tg cultures, 14.0±2.0%, p<0.0001; 21 DIV: wild-type cultures, 3.0±0.2%; Tg cultures, 24.0±5.0%; p<0.0001, wild-type vs Tg; p=0.0003, 14 vs 21 DIV). n=50 cells from each condition.

FIGS. 2A to 2F show abnormal morphologies in neurons from Tg cultures at 21 DIV. FIGS. 2A and 2B show representative images of wild-type or Tg GFP-labeled mature neurons (21 DIV). FIG. 2C shows sholl analysis of total number of branch point on basal dendrites of neuron from wild-type and Tg cultures, indicating that decreased complexity in Tg neurons starting at 30 μm from the cell body. n=25 cells from each condition. FIGS. 2D and 2E show representative GFP-labeled dendritic segments studded with mature spines from wild-type and Tg neurons, indicating a loss of spines on Tg dendrites. FIG. 2F shows quantitative results of spine densities determined in neurons without apparent dystrophies at 21 DIV in wild-type and Tg cultures. The results confirmed the observation shown in FIGS. 2D and 2E. n=4 culture per experiment and 400 spines from each condition. *p<0.05; **p<0.01; data represent mean±SD.

FIG. 3 shows the level of neuron viability of wild-type and Tg neurons at 7 DIV and 14 DIV detected by ToxiLight Bioassay. Data represent the mean±SEM.

FIG. 4A to 4D show levels of amyloid-beta in conditioned media from Tg and wild-type cultures, and levels of Ca²⁺ for various indicated conditions. FIGS. 4A and 4B shows analysis of Aβ 40 and Aβ42 in medium from Tg2576 cultures. Both Aβ40 and Aβ42 levels from Tg2576 cultured medium at different dates were measured using ELISA. Both levels of Aβ40 and Aβ42 increased during neuron maturation; Aβ40 levels increased 25-fold from 0 DIV to 14 DIV, then slightly decreased to 14-fold at 21 DIV; Aβ42 levels increased 5-fold from 0 DIV to 14 DIV, then slightly decreased to 3-fold at 21 DIV. FIG. 4C shows that different Aβ species were detected in Tg cultured medium collected from 14 DIV cultures by immunoprecipitation. A total of 1.3 ml medium were immunoprecipitated with 5 μg 6E10 antibody and immunoblotted with 6E10 antibody for determination of Aβ. Arrows indicated visible bands which sizes are corresponded to different Aβ species as indicated. FIG. 4D shows [Ca²⁺]ᵢ measurements of various indicated conditions. Calcium concentrations were determined in 13DIV wild type neurons treated with either wtCM or TgCM for 24 h and incubated with Indo-1/AM the next day. Wild type neurons treated with TgCM showed significantly higher calcium levels (115 nM±1, n=510 cells) compared with cells treated with wtCM (83 nM±1, n=450 cells). This increase in calcium concentration was partially prevented when Aβ peptides were depleted of TgCM with 3D6 antibody (98±1 nM, n=340 cells). Treatment with wtCM did not change resting calcium levels compared with untreated cultures (Untreated; 84±1; n=170 cells). The symbol “*” indicated a p-value of less than 0.0001, ANOVA and post hoc student test.

FIGS. 5A to 5G show that Aβ induces NFATc4 and (calcineurin) CaN-aberrant nuclear localization in Tg neurons in culture and AD postmortem brains. FIG. 5A shows quantification results of NFATc4 nuclear immunoreactivity from immunostaining images of NFATc4 in Tg or wild-type neurons at 14 DIV, which were labeled for NFATc4, MAP2, and Hoechst nuclear counterstain (images not shown). n=40 cells from 3 different experiments. The symbol “*” indicates a p-values of less than 0.05; data represent mean±SD. FIG. 5B shows quantification results of CaN nuclear immunoreactivity from immunostaining images of CaN in Tg or wild-type neurons at 14 DIV, which were labeled for CaN and Hoechst nuclear counterstain (images not shown). Compared with wild-type neurons, Tg neurons show higher immunoreactivity of CaN in the nuclei. n=40. The symbol “**” indicates a p-value of less than 0.01; data represent mean±SD. FIGS. 5C and 5D shows western blot analysis of NFATc4 and HDAC in the brains of AD patients. Subcellular fractions were prepared from brains of AD patients or controls and analyzed by immunoblotting for NFATc4 and cytoplasmic or nuclear control proteins GAPDH and HDAC1, respectively. FIG. 5E shows quantification results of NFATc4 immunoreactivity from the western blot image in FIG. 5C. NFATc4 was detected in all three compartments but substantially enriched in nuclei in AD brains (n=6 for each condition). The symbol “*” indicates a p-value of less than 0.05; data represent mean±SD. FIGS. 5F and 5G show quantification results of immunoreactivity from each subcellular fraction for both full-length CaN (60 kDa) and CaNCA (45 kDa), respectively. Inset in FIG. 5G shows representative blots of CaN in nuclear fraction. The symbol “*” indicates a p-value of less than 0.05; data represent mean±SEM.

FIGS. 6A to 6G show that conditioned medium induces NFATc4-aberrant nuclear translocation in wild-type cultured neurons. FIGS. 6A and 6B show a schematic representation of AKAP79 inhibitory peptide for CaN and AAV2 viral vectors (AAV-CMV/CBA-WPRE) with AKAP79 inhibitory peptide, respectively. FIG. 6C shows quantification results of NFATc4-aberrant nuclear translocation induced by TgCM in wild-type cultured neurons. The ratio of nucleus to cytoplasm is shown. TgCM causes an increase in the nucleus/cytoplasm ratio of NFATc4 that is dependent on the presence of Aβ (because anti-Aβ antibody 3D6 prevents the effect). Inhibition of CaN activation by AKAP79 peptide also prevented the increase in the nucleus/cytoplasm ratio of NFATc4 (n>40 cells). Data represent mean±SD. The symbol of “*” indicates a p-value of less than 0.05. FIG. 6D shows a representative immunoblot indicating separation of oligomeric Aβ from TgCM by size-exclusion chromatography. sAPP separated in fractions 6 and 7 (sAPP fraction, immunolabeled by 6E10) and Aβ separated in fractions 18 and 19 (Aβ fraction, immunolabeled by 82E1). Arrowheads showed estimated molecular mass. FIGS. 6E and 6F show quantification results of Aβ40 and Aβ42 in SEC-separated fractions form TgCM and wtCM by ELISA, respectively. Fractions 18 and 19 of TgCM contained 451.7 pM and 582.0 pM of Aβ40 and 28.2 pM and 34.4 pM of Aβ42, respectively. Fraction 18 and 19 of wtCM contained 9.1 pM and 5.0 pM of Aβ40 and 0.0 pM and 0.4 pM of Aβ42, respectively. FIG. 6G shows quantification of NFATc4-aberrant nuclear translocation induced by different SEC fractions in wild-type cultured neurons. Application of SEC fractions 6-7 (sAPP fraction) from either TgCM or wtCM caused no significant difference on the nucleus/cytoplasm ratio of NFATc4. However, application of SEC fractions 18-19 (Aβfraction) of TgCM onto wild-type neurons for 24 h caused significant increase in translocation of NFATc4 to the nucleus, but no changes were observed in neurons applied with the same SEC fractions of wtCM. Immunodepletion of Aβ from the fractions 18-19 of TgCM with 3D6 prevented the increase in the nucleus/cytoplasm ratio of NFATc4. n>35 cells. Data represent mean±SD; the symbol “*” indicates a p-value of less than 0.05. ITR is short for “Inverted Terminal Repeat.”

FIGS. 7A to 7H show that aberrant neuronal morphologies induced by TgCM or APP overexpression are prevented by Aβ depletion. FIG. 7A shows the percentage of neurons with dendritic dystrophies at 21 DIV at various indicated conditions. Primary cultures were maintained in TgCM for 21 DIV in different culture conditions, as indicated. The percentage of neurons with dendritic dystrophies at 21 DIV is increased in the presence of TgCM, and this beading can be prevented by immunodepletion of Aβ. FIG. 7B and FIG. 7C show representative GFP-labeled mature neurons and Sholl analysis of branching on dendrites of neurons from indicated conditions, respectively, indicating that TgCM reduced branching and that this is rescued by immunodepletion of Aβ with 3D6. FIG. 7D shows images and quantification results of spine densities determined in neurons without apparent dystrophies in each experimental condition. The results indicate that a decrease in spine density with TgCM could be rescued with 3D6 treatment. FIGS. 7E to 7H show that Aβ depletion prevents APP overexpression-induced morphological abnormalities. In Tg cultures at 21 DIV, dendritic dystrophies (FIG. 7E), dendritic attenuation (FIGS. 7F and 7G) and spine loss (FIG. 7H) are prevented by Aβ depletion with 3D6. Sixty cells were analyzed per experimental condition in each experiment. The symbol “*” indicates a p-value of less than 0.05; the symbol “*” indicates a p-value of less than 0.01; and the symbol “*” in FIG. 7G indicates a p-value of less than 0.05 (Tg with 3D6 vs Tg with boiled 3D6). Data represent mean±SD.

FIGS. 8A to 8I show that CaN inhibition by AKAP79 inhibitory peptide prevents TgCM-induced morphological abnormalities. Dendritic dystrophies (FIG. 8A), dendritic attenuation (FIGS. 8B and 8C), and spine loss (FIG. 8D) in wild-type cultured neurons growing in TgCM are significantly prevented by CaN inhibitory peptide AKAP79. The symbol “*” indicates a p-value of less than 0.05 in FIG. 8C (TgCM with AKAP79 vs TgCM with vector). The symbol “*” indicates a p-value of less than 0.01. Data are mean±SD from three independent experiments in triplicate. FIGS. 8E to 8H show that CaN inhibition by AKAP79 inhibitory peptide prevents APP overexpression-induced morphological abnormalities. In Tg cultures at 21 DIV, dendritic dystrophies (FIG. 8E), dendritic attenuation (FIGS. 8F and 8G), and spine loss (FIG. 8H) are prevented by CaN inhibitory peptide AKAP79. The symbol “*” indicates a p-value of less than 0.05 in FIG. 8G (Tg with AKAP79 vs Tg with vector). Data are mean±SD from three independent experiments, each in triplicate. FIG. 8I shows that CaN inhibition or Aβ immunodepletion prevents Tg CM induced morphological abnormalities. A decreased spine density was observed when primary neurons were treated with TgCM for 24 h compared with wtCM treated cells. The spine density was restored when neurons were transfected with AKAP79 or cultured in depleted TgCM or TgCM containing either 1 μM FK506 or 2 μM VIVIT (SEQ ID NO: 7).

FIGS. 9A to 9I show that inhibition of CaN-NFAT interaction by VIVIT (SEQ ID NO: 7) prevents TgCM-induced morphological abnormalities. FIG. 9A shows analysis of endogenous NFATc4 distribution. The repartition of NFATc4 was analyzed by measuring the ratio between nuclear and cytoplasmic signals. The symbol “*” indicates a p-value of less than 0.001. ANOVA and post-hoc Student's test. (The symbol “*” indicates a p-value of less than 0.05). n=40 cells for each condition from two independent experiments. FIGS. 9B to 9E show images and quantification results of neurodegenerative morphologies observed in wild-type cultures neurons growing in TgCM. Dendritic dystrophies (FIG. 9B), dendritic attenuation (FIGS. 9C and 9D), and spine loss (FIG. 9E) in wild-type cultured neurons growing in TgCM are significantly prevented by VIVIT (SEQ ID NO: 7) (catalog #480401; Calbiochem). The symbol “*” indicates a p-value of less than 0.05 in FIG. 9D (TgCM with VIVIT (SEQ ID NO: 7) vs TgCM with DMSO). Data are mean±SD from three independent experiments in triplicate. FIGS. 9F to 9I show images and quantification results of morphological abnormalities observed in each indicated condition. Inhibition of CaN-NFAT interaction by VIVIT (SEQ ID NO: 7) prevents APP overexpression-induced morphological abnormalities. In Tg cultures at 21 DIV, dendritic dystrophies (FIG. 9F), dendritic attenuation (FIGS. 9G and 9H), and spine loss (FIG. 9I) are prevented by VIVIT (SEQ ID NO: 7). The symbol “*” indicates a p-value of less than 0.05 in FIG. 9H (Tg with VIVIT (SEQ ID NO: 7) vs Tg with DMSO). Data are mean±SD from three independent experiments in triplicate.

FIGS. 10A to 10H show wild-type cultured neurons overexpressing a constitutively active CaN construct, CaNCA, develop abnormal morphology that is a phenocopy of the AD effect. FIG. 10A shows the results of NFAT reporter activity of neuronal cells under various indicated conditions. Wild-type cultured neurons were infected with AAV-CaNwt or AAV-CaNCA with or without AAV-flag-AKAP79 for twenty-four hours, and then the cell extracts were assayed for luciferase activity. The symbol “*” indicates a p-value of less than 0.05; Data are mean±SD (n=3 experiments each). FIGS. 10B to 10G show images and quantification results of neurodegenerative morphologies observed in wild-type cultured neurons overexpressing CaNCA or a vector. Overexpression of CaNCA, but not CaNwt or vector control, into wild-type cultures induces dendritic dystrophies (FIGS. 10B and 10C; arrows in FIG. 10B indicated local swelling of dendrites), simplification of dendritic arborization (FIGS. 10D and 10E), and spine loss (FIGS. 10F and 10G). Neurons expressing CaNCA displayed significantly more neurons with dendritic dystrophies, simplified dendritic complexity, and spine loss than either CaNwt or vector-expressing neurons. The symbol “*” indicates a p-value of less than 0.05; The symbol “*” in FIG. 10E indicates a p-value of less than 0.05 (CaNwt vs CaNCA). Data are mean±SD from three independent experiments, each in triplicate. FIG. 10H shows spine density of 15 DIV primary neurons transfected with wtCaN+GFP (with DMSO), CaNCA+GFP (with DMSO or VIVIT (SEQ ID NO: 7)) and CaNCA+GFP+AKAP79. Neurons were transfected at 10DIV and analyzed at 14DIV. A decreased spines density spines is observed when CaCA is overexpressed (15.2±1.1) compared with wtCaN (25.9±2.1). This effect was blocked by cotransfection with AKAP79 (25.6±1.0) or by the addition of VIVIT (SEQ ID NO: 7) in the medium (22±1.7). The symbol “**” indicates a p-value of less than 0.001 (ANOVA) and less than 0.05 (post-hoc Student's test). n≧15.

FIG. 11 shows the quantitative analysis of spine densities from live imaging of neurons without apparent dystrophie (image not shown). Overexpression of CaNCA, but not CaNwt, in the intact mouse brain induces abnormal morphologies, resulting in a decrease in spine density.

FIG. 12A to 12D show that abnormal morphologies are prevented by overexpression of CaN inhibitory peptide AKAP79 in APP/PS1 mouse brain. FIGS. 12A, 12B, and 12C show quantification of spine density, dendritic dystrophies, and neurite curvature, respectively, near Aβ deposits in vector- or AKAP79-expressing APP/PS1 mouse brain. FIG. 12A shows that AKAP79 expression decreases dystrophy size (vector, 16.4±6.1 μm2; AKAP79, 12.8±3.6; p=0.029; n=45 from 4 animals). FIG. 12B shows that AKAP79 expression increases spine density (vector, 17.8±3.0/100 μm; AKAP79, 28.3±6.1; p<0.001; n>400 spines). FIG. 12C shows that AKAP79 expression decreases abnormal neurite curvature near plaques (n=4 animals for each condition and total >400 spines). FIG. 12D shows quantification of numbers of axonal dystrophies per single Aβ deposits from postmortem sections. Values represent mean±SD. The symbol “*” indicates a p-value of less than 0.05; while the symbol “**” indicates a p-value of less than 0.01.

FIG. 13A to 13E shows quantification of spine density in vivo under various conditions. FIG. 13A shows that the overexpression of VIVIT-GFP and Nls-VIVIT (co-injected with GFP) in neurons surrounding amyloidplaques improves spine density compared with GFP injected transgenic animals. In APP/PS mice, the spine density was evaluated for neurites that were less than 90 μm far from an amyloid plaque. n=4 APP/PS mice injected with either GFP or VIVIT-GFP and n=3 littermates injected with GFP and APP/PS injected with nls-VIVIT (more than 75 neurites were analyzed for each condition and more than 4000 spines were counted). ANOVA (P<0.001) and post-hoc Student T-Test (P<0.05). FIG. 13B shows that in APP/PS injected with an AAV-GFP vector, a linear correlation was observed between the spine density and the distance of the neurite from amyloid plaque. FIG. 13C shows that no correlation could be found anymore when the AAV-VIVIT vector was injected. FIG. 13D shows that the linear relationship observed in FIG. 13B is partially abolished when the nls-VIVIT vector was overexpressed. FIG. 13E contains all the results pooled from FIGS. 13B to 13D. n=4 APP/PS mice injected with either GFP or VIVIT-GFP and n=3 APP/PS injected with nls-VIVIT.

### DETAILED DESCRIPTION OF THE INVENTION

Alzheimer's disease (AD) is a progressive neurodegenerative disorder and the leading cause of dementia in the elderly. As the incidence and prevalence of AD rise steadily with increasing longevity, AD threatens to become a catastrophic burden on health care. Yet there are no effective therapeutic inventions for AD treatment.

In accordance with aspects of the invention, activation of calcineurin (CaN)-mediated nuclear factor of activated T cells (NFAT) can produce similar phenotypes of amyloid-beta (Aβ)-induced neurodegenerative alterations in neurons, e.g., dystrophic neuritis, dendritic simplification, and dendritic spine loss, in both in vitro cultures and in in vivo adult mouse brains. The inventors have demonstrated that inhibition of CaN-NFAT interaction, e.g., with a peptide comprising VIVIT (SEQ ID NO: 7), reduces neuritic dystrophies and/or improves dendritic spine density in neurons in vitro and in an in vivo mouse AD model. Accordingly, some embodiments of the invention are generally related to methods and compositions for inhibiting neurodegeneration, e.g., in vitro, ex vivo or in vivo. Another aspect of the invention relates to methods and compositions for treating a neurodegenerative disorder, e.g., Alzheimer's disease.

One aspect of the invention provides a method for inhibiting neurodegeneration, i.e., any condition in which neuronal structure or function is reduced, including death of neurons. Many neurodegenerative diseases including Parkinson's, Alzheimer's, and Huntington's occur as a result of neurodegenerative processes. By “neurodegenerative disorder” is meant any disease or disorder caused by or associated with the deterioration of neurons. Exemplary neurodegenerative disorders are polyglutamine expansion disorders (e.g., HD, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, and Tabes dorsalis.

Methods of the invention are also useful for the treatment of neuronal damage caused by encephalitis, brain injury, inflammation-induced neurodegeneration or tau-induced synaptic neurodegeneration.

In some embodiments, the methods of the invention can be used for treatment of tau-induced synaptic neurodegeneration.

In some embodiments, the methods of the invention can be used for treatment of neurodegeneration caused by encephalitis or brain injury.

In one embodiment, the methods of the invention are used for treatment of amyloid-beta-induced neurodegeneration.

In one embodiment, the methods of the invention are used for treatment of frontotemproal dementia.

In some embodiments, the methods of the invention can be used for any condition, where synapse strengthening is in need.

In one aspect, the method for inhibiting neurodegeneration includes comprising a population of neuronal cells with an effective amount of a nuclear factor of activated T cells (NFAT) antagonist. As used herein, the term “contacting” refers to any suitable means for delivering, or exposing, an agent to at least one cell. Exemplary delivery methods include, but are not limited to, direct delivery to cell culture medium, delivery to an in vitro scaffold in which cells are seeded, e.g., via perfusion or injection, or other delivery method well known to one skilled in the art. In one embodiment, the contacting is in vitro, e.g., a NFAT antagonist is added to the cell culture medium in which neuronal cells are cultured. In still another embodiment, NFAT antagonist is injected into a biocompatible gel (e.g., peptide gel, hydrogel) in which neuronal cells are encapsulated. In some embodiments, the contacting can be ex vivo. The term “ex vivo” refers to a condition where biological materials, typically cells, are obtained from a subject or a suitable alternate source, such as, a suitable donor, and are modified, such that the modified cells can be used to treat a pathological condition which will be improved by the long-term or constant delivery of the therapeutic benefit produced by the modified cells. For example, neuronal cells, e.g., neuronal stem cells, obtained from a subject or an alternative source can be treated with a NFAT antagonist for inhibition of neurodegeneration, and then re-introduced into the subject. A benefit of ex vivo therapy is the ability to provide the patient the benefit of the treatment, without exposing the patient to undesired collateral effects from the treatment. The term “treatment” or “treated” in reference to exposing cells to an agent, e.g., treatment of neuronal cells with a NFAT antagonist, is used herein interchangeably with the term “contacting”. In some embodiments, the contacting can be in vivo, e.g., in a subject diagnosed with or predisposed to a neurodegenerative disorder, e.g., Alzheimer's disease. In some embodiments, the in vivo contacting can be performed by injection, e.g., intracortical injection. Other forms of administration can also be employed in methods of the invention, e.g., systemic, oral, or parenteral administration. One of skill in the art can determine an appropriate administration method known in the art according to various embodiments of the invention.

In some embodiments, the population of neuronal cells described herein can be contacted more than once with at least one NFAT antagonist. In some embodiments, the neuronal cells can be contacted with one or more NFAT antagonists at least twice, at least three times, at least four times, or at least five times. A different NFAT antagonist or a combination thereof can be used in each cell treatment.

In some embodiments, the neuronal cells can be contacted with at least one additional agent prior to, concurrent with, or after administration. Such agents can be any therapeutic agents for treatment of neurodegeneration or a neurodegenerative disorder, cytokines, an additional NFAT antagonist or a mixture thereof.

The neuronal cells can be contacted with a NFAT antagonist via different means, based upon the contacting condition. In one embodiment, the contacting condition is in vivo, e.g., contacting the neuronal cells in the brain of a subject. In such embodiments, the contacting can be performed by injection. In one embodiment, the injection is intracortical. Alternatively, the neuronal cells can be contacted with a NFAT antagonist by intracranial injection. In one embodiment, a catheter-based approach is used for the purpose of the invention. The use of a catheter precludes more invasive methods of delivery wherein the opening of the brain would be necessitated. As one skilled in the art would appreciate, optimum time of recovery would be allowed by the more minimally invasive procedure. A catheter approach can involve the use of such techniques as the NOGA catheter or similar systems. The NOGA catheter system facilitates guided administration by providing electromechanic mapping of the area of interest, as well as a retractable needle that can be used to deliver targeted injections or to bathe a targeted area with a therapeutic. Any methods of the invention can be performed through the use of such a system to deliver injections. One of skill in the art will recognize alternate systems that also provide the ability to provide targeted treatment through the integration of imaging and a catheter delivery system that can be used with the methods of the invention. One of skill in the art will also recognize other useful methods of delivery or implantation which can be utilized with the methods of the invention.

In embodiments of the invention, contacting neuronal cells with a NFAT antagonist can result in amelioration of at least one symptom associated with neurodegeneration, e.g., a decrease in cognitive function. In one embodiment, at least one symptom of neurodegeneration is alleviated by at least about 5%, at least about 10%, at least about 15%, at least about 20%, at least about 30%, at least about 40%, or at least about 50%. In one embodiment, at least one symptom is alleviated by more than 50%. In one embodiment, at least one symptom is alleviated by at least about 80%, at least about 90% or greater, as compared to the severity of symptoms in the absence of a NFAT antagonist.

Neuronal Cells

The term “neuronal cells” or “neurons” as used herein refers to cells that express one or more neuron-specific markers. Examples of such markers can include, but are not limited to, neurofilament, microtubule-associated protein-2, tau protein, neuron-specific Class III β-tubulin, and NeuN. In some embodiments, neuronal cells can include cells that are post-mitotic and express one or more neuron-specific markers.

In the brain, there are both neurons and support (non-neuronal) cells such as glia cells. The term “population” as used herein refers to more than one cell with the same phenotypic characteristics. The phrase “a population of neuronal cells” as used herein refers to a collection of cells, in which one or more cells express at least one neuron-specific markers, for example, at least about 10% neuronal cells, at least about 20% neuronal cells, at least about 30% neuronal cells, at least about 40% neuronal cells, at least about 50% neuronal cells, at least about 60% neuronal cells, at least about 70% neuronal cells, at least about 80% neuronal cells, at least about 90% neuronal cells, at least about 95%, about 98%, about 99% or 100% neuronal cells. Other cells that can be present in a population of neuronal cells can include, but are not limited to, non-neuronal cells, e.g., glia cells, or any cells in the brain tissue. Glia cells include oligodendrocytes, microglia, and astrocytes, each of which is needed to optimize brain function.

Nuclear Factor of Activated T Cells (NFAT) Antagonist and Viral Vectors

The NFAT family consists of five members: NFAT1 (also known as NFATp or NFATc2), NFAT2 (also known as NFATc or NFATc1), NFAT3 (also known as NFATc4), NFAT4 (also known as NFATx or NFATc3), and NFAT5. Four of these proteins, except NFAT5, are regulated by calcium signaling. Each protein can have two or more alternatively spliced forms; splicing results in variation at the amino (N) and carboxyl (C) termini with the core region being conserved. Vihma H et al., Genomics 2008. November 92 (5) 279-291. The conserved core region of NFAT proteins consists of two tandem domains: a regulatory domain, which is also known as the NFAT-homology region (NHR); and the Rel-homology region (RHR), which binds DNA. The NHR is moderately conserved among NFAT proteins and contains a transactivation domain. The NHR contains many serine residues that are phosphorylated in resting T cells. It also includes the docking sites for calcineurin and the NFAT kinases, which regulate the activation of NFAT proteins by determining the phosphorylation status of the serines. The RHR domain shares structural homology with REL proteins and confers the DNA-binding specificity that characterizes NFAT family members. NFAT3 has a similar domain structure, but it is mainly expressed outside the immune system. Macian F. 5 Nature Reviews: Immunology 472 (2005).

NFAT is one of the substrates for calcineurin. Ca²⁺ binds calmodulin, which in turn activates the calmodulin-dependent phosphatase calcineurin. NFAT proteins are dephosphorylated by activated calcineurin, which leads to their nuclear translocation and the induction of NFAT-mediated gene transcription. These NFAT transcription factors tend to reside in the cytosol in a highly phosphorylated state when intracellular Ca²⁺ levels are low, but are bound tightly by activated CaN and dephosphorylated when Ca²⁺ levels rise. CaN-mediated dephosphorylation of NFATs reveals a nuclear import sequence (or a nuclear localization sequence) which permits transport into the nucleus. Once in the nucleus, NFATs interact with specific DNA binding elements to regulate gene expression in conjunction with other transcription factors (e.g. AP1, MEFs, and NFκB). Conversely, a nuclear export sequence is exposed and NFATs are shuttled back to the cytosol upon re-phosphorylation by a variety of protein kinases (e.g. glycogen synthase kinase 3β). Macian F. 5 Nature Reviews: Immunology 472 (2005), and Abdul H. M. et al., 2 Mol Cell Pharmacol 7 (2010).

The term “antagonist” as used herein refers to a molecule which is capable of decreasing one or more of the biological activities of a target molecule, such as an NFAT. Antagonists may, for example, act by inhibiting a target molecule and/or mediating signal transduction. In some embodiments, the NFAT inhibitor can decrease the expression of NFAT. In some embodiments, the NFAT inhibitor can inhibit NFAT activation, e.g., by inhibiting dephosphorylation and/or nuclear translocation of NFAT. In some embodiments, the NFAT inhibitor can inhibit interaction of CaN with NFAT, e.g., by blocking the CaN-binding domain of NFAT. In some embodiments, the NFAT inhibitor can compete with NFAT protein for CaN.

A NFAT antagonist can include any molecule that acts as antagonist against NFAT activation. Such NFAT antagonists include, but are not limited to, a small molecule, a peptide, a protein, an antibody, and a nucleic acid. For example, NFAT inhibitors can be derived using NFAT and/or CaN nucleic acid or amino acid sequences. The nucleotide and amino acid sequences of these molecules are known in the art and can be found in NCBI Entrez Gene database. See, for example, NFATc4 has been assigned a NCBI accession number for different species such as human, mouse and rat. By way of example, the NCBI accession numbers for the nucleotide and amino acid sequences of human NFATc4 are NM_{—}001136022 and NP_{—}001129494, respectively. One of ordinary skill can find the amino acid and nucleotide sequences of various NFAT family members and/or CaN to design a NFAT inhibitor accordingly.

In embodiments of the invention, general inhibitors of NFAT can be used, i.e., antagonists that inhibit more than one NFAT. In one embodiment, the NFAT antagonist is specific NFAT1. In one embodiment, the NFAT antagonist is specific NFAT2. In one embodiment, the NFAT antagonist is specific NFAT3. In one embodiment, the NFAT antagonist is specific NFAT4. In one embodiment, the NFAT antagonist is specific NFAT5.

In one embodiment, the NFAT antagonist is a direct inhibitor of NFAT. As used herein, the phrase “direct inhibitor” refers to an inhibitor that physically interacts with NFAT and e.g. physically disrupts the interaction between NFAT and calcineurin, or alternatively the direct inhibitor inhibits NFAT binding to downstream effectors or genes. In one embodiment, the direct inhibitor inhibits binding of NFAT to calcineurin. In certain embodiments, the direct inhibitor inhibits binding of NFAT to calcineurin by at least about 5%, at least about 10%, at least about 20%, at least about 30%, at least about 40%, at least about 50%, at least about 60%, at least about 70%, at least about 80%, at least about 90%, at least about 95%, about 96%, about 97%, about 98%, about 99% or about 100%. Assays to measure binding between a NFAT antagonist and NFAT, and its antagonistic effect are well known to those of skill in the art.

In certain embodiments the direct inhibitor inhibits NFAT binding to downstream effectors or genes by at least about 5%, at least about 10%, at least about 20%, at least about 30%, at least about 40%, at least about 50%, at least about 60%, at least about 70%, at least about 80%, at least about 90%, at least about 95%, about 96%, about 97%, about 98%, about 99% or about 100%. Inhibition of binding to downstream effectors or genes inhibits transcriptional activation. Inhibition of transcriptional activation can be assayed using general transcriptional activation reporting assays known to those of skill in the art. For example, transcriptional activation by NFAT can be measured using vectors comprising neuronal gene promoters that are activated by NFAT which are operably linked to reporter genes.

Small Molecules:

In various embodiments, the NFAT inhibitor can be a small molecule, e.g., pyrazolopyrimidine compound NCI3, as described in Sieber M. 37 Eur J. Immunol. 2617 (2007). Small molecules of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In accordance with the invention, inhibition of calcineurin leads to an inactivation of NFAT, e.g. a phosphorylation of NFAT. Accordingly, calcineurin inhibitors can also be used as NFAT inhibitors for the purpose of the invention. Non-limiting examples of calcineurin inhibitors include cyclosporin A (Novartis International AG, Switzerland), tacrolimus (FK506) (Fujisawa Healthcare, Inc., Deerfield, Ill., USA), FK520 (Merck & Co, Rathway, N.J., USA), L685,818 and L732/731 (Merck & Co), ISATX247, (Hoffman-La Roche Ltd), FK523, 15-0-DeMe-FK-520 (Liu, Biochemistry, 31:3896-3902 (1992)), and the ones disclosed in the patent applications. For example, WO2005087798 describes cyclosporine derivative inhibiting calcineurin, and WO2006078724 describes FK506 and FK520 analogs inhibiting calcineurin.

Proteins and Peptides:

NFAT antagonistic peptides can be used for the purpose of the invention. A peptide can be a fragment of the naturally occurring protein, or a mimic or peptidomimetic of NFAT, e.g., a CaN-binding domain of NFAT. Variants of NFAT antagonistic peptides can be generated by mutagenesis (e.g., amino acid substitution, amino acid insertion, or truncation), and identified by screening combinatorial libraries of mutants, such as truncation mutants, for the desired activity.

Calcineurin-mediated dephosphorylation requires docking of calcineurin on NFAT. Interactions between NFAT and calcineurin occur at a specific motif in the N terminus of NFAT, which has the consensus sequence PXIXIT, where X denotes any amino acid. This motif is conserved among different NFAT family members and constitutes the main docking site for calcineurin on NFAT. A high-affinity version of this peptide, VIVIT (SEQ ID NO: 7), was shown to compete with NFAT proteins for calcineurin binding and to block NFAT dephosphorylation in vitro. Macian F. 5 Nature Reviews: Immunology 472 (2005). Accordingly, in some embodiments, the NFAT inhibitor can be a peptide comprising an amino acid sequence of XIXIT, where X denotes any amino acid. In one embodiment, the NFAT inhibitor can be a peptide comprising an amino acid sequence of VIVIT (SEQ ID NO: 7).

The NFAT antagonistic peptide, e.g., a VIVIT-containing peptide, can be produced recombinantly or direct chemical synthesis. Further, the peptide can be produced as a modified peptide, with nonpeptide moieties attached, e.g., by covalent linkage, to the N-terminus and/or C-terminus. In some embodiments, either the carboxy-terminus or the amino-terminus, or both, can be chemically modified. Some common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can change physical, chemical, biochemical, and pharmacological properties, such as: increased nuclear import, enhanced stability, cell permeability, increased potency and/or efficacy, resistance to serum proteases, and desirable pharmacokinetic properties.

In some embodiments, the NFAT antagonistic peptide, e.g., a VIVIT-containing peptide, can further comprise a nuclear localization sequence or signal (NLS). A nuclear localization signal or sequence (NLS) is an amino acid sequence which is used to target the protein to the cell nucleus through the Nuclear Pore Complex and/or to direct a newly synthesized protein into the nucleus via its recognition by cytosolic nuclear transport receptors. Typically, this signal consists of one or more short sequences of positively charged lysines or arginines. NFAT contains two NLS motifs: one in the NHR domain and one in the RHR domain. However, the latter is less efficient in translocating NFAT to the nucleus. Janeway, C. A., et al., Immunology. Garland Publishing. 205 (2001).

In some embodiments, the NFAT antagonistic peptide, e.g., a VIVIT-containing peptide, can be modified at the N-terminal, e.g., with an eleven arginine transduction domain and a three glycine linker sequence, to make the peptide cell-permeable. Any commerically-available NFAT inhibitors, e.g., different variants of VIVIT-containing peptides from Tocris Bioscience or EMD Biosciences, can be also employed in the methods of the invention.

In some embodiments of the invention, the NFAT inhibitor can be a peptide analog or peptide mimetic thereof. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15:29; Veber and Freidinger (1985) TINS p. 392; and Evans et al. (1987) J. Med. Chem. 30:1229, which are incorporated herein by reference) and are usually developed with the aid of computerized molecular modeling. Peptide mimetics that are structurally similar to the NFAT inhibitor or functional variants thereof can be used to produce an antagonistic effect. Generally, peptidomimetics are structurally similar to the paradigm polypeptide (e.g., a VIVIT (SEQ ID NO: 7) peptide) but have one or more peptide linkages optionally replaced by a linkage selected from the group consisting of: —CH2NH—, —CH2S—, —CH2-CH2-, —CH═CH—(cis and trans), —COCH2-, —CH(OH)CH2-, and —CH2SO—. This is accomplished by the skilled practitioner by methods known in the art which are further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference.

In some embodiments, protein inhibitors which prevents NFAT nuclear translocation can be used as NFAT inhibitors. In some embodiments, these protein inhibitors can bind calcineurin, resulting in inhibiting NFAT nuclear translocation. Such protein inhibitors include, but are not limited to, AKAP79 (a scaffold protein that prevents calcineurin substrate interactions), CABIN protein (which blocks calcineurin activity), a calcineurin B homolog, CHP, and MCIP1,2,3 proteins which have the ability to prevent NFAT2 phosphorylation and nuclear import (Crabtree et al., 2002).

Intrabodies:

In another embodiment, the invention employs intrabodies to inhibit NFAT activation or its interaction with CaN, e.g, inhibit NFAT dephosphorylation. As used herein, the term “intrabody” is an antibody that works within the cell to bind to an intracellular protein. In some embodiments, intrabodies refer to antibodies that have been modified for intracellular localization. The term “intrabodies” can apply to several types of protein targeting: the antibody may remain in the cytoplasm, or it may have a nuclear localization signal, or it may undergo co-translational translocation across the membrane into the lumen of the endoplasmic reticulum, provided that it is retained in that compartment, e.g., through a KDEL sequence.

In some embodiments, intrabodies can include whole antibodies or antigen-binding fragments thereof including, for example, Fab, F(ab′)₂, Fv and single chain Fv fragments. Suitable antibodies include any form of antibody, e.g., murine, human, chimeric, or humanized and any type antibody isotype, such as IgG1, IgG2, IgG3, IgG4, IgM, IgA1, IgA2, IgAsec, IgD, or IgE isotypes.

Because antibodies ordinarily are designed to be secreted from the cell, in some embodiments, intrabodies can require special alterations, including the use of single-chain antibodies (scFvs), modification of immunoglobulin VL domains for hyperstability, selection of antibodies resistant to the more reducing intracellular environment, or expression as a fusion protein with maltose binding protein or other stable intracellular proteins. Such optimizations can improve the stability and structure of intrabodies.

NFAT intrabodies can be produced according to well known methods for intrabody production, e.g., the method described in the PCT patent application: WO 2002/086096, and tested for antagonist activity using the methods described herein. For example, antigenic peptides of NFAT which are useful for the generation of intrabodies can be identified in a variety of manners well known in the art. By way of example, useful epitopes can be predicted by analyzing the sequence of the NFAT protein using predictive algorithms known in the art to generate potential antigenic peptides from which synthetic versions can be made and tested for their capacity to generate NFAT-specific intrabodies.

The NFAT intrabodies can be monoclonal or polyclonal. The terms “monoclonal intrabodies” as used herein, refers to a population of intrabody molecules that contain only one species of an antigen binding site capable of immunoreacting with a particular epitope of an antigen, whereas the term “polyclonal intrabodies” refers to a population of intrabody molecules that contain multiple species of antigen binding sites capable of interacting with a particular antigen. Techniques for generating monoclonal and polyclonal intrabodies are well known in the art (See, e.g., Current Protocols in Immunology, Coligan et al., eds., John Wiley & Sons, http://www.does.org/masterli/cpi.html).

Recombinant NFAT intrabodies, such as chimeric and humanized monoclonal intrabodies, comprising both human and non-human portions can be made using standard recombinant DNA techniques, and are also within the scope of the invention.

Recombinant chimeric intrabodies can be further humanized by replacing sequences of the Fv variable region which are not directly involved in antigen binding with equivalent sequences from human Fv variable regions. General reviews of humanized chimeric antibodies are provided by Morrison, S. L., 1985, Science 229:1202-1207 and by Oi et al., 1986, BioTechniques 4:214. Those methods include isolating, manipulating, and expressing the nucleic acid sequences that encode all or part of immunoglobulin Fv variable regions from at least one of a heavy or light chain. Sources of such nucleic acid are well known to those skilled in the art. The recombinant DNA encoding the chimeric intrabody, or fragment thereof, can then be cloned into an appropriate expression vector. Suitable humanized intrabodies can alternatively be produced by CDR substitution U.S. Pat. No. 5,225,539; Jones et al. 1986 Nature 321:552-525; Verhoeyan et al. 1988 Science 239:1534; and Beidler et al. 1988 J. Immunol. 141:4053-4060.

Chimeric and humanized intrabodies in which specific amino acids have been substituted, deleted or added are also within the scope of the invention. In some embodiments, humanized intrabodies have amino acid substitutions in the framework region, such as to improve binding to the antigen. For example, in a humanized intrabody having mouse CDRs, amino acids located in the human framework region can be replaced with the amino acids located at the corresponding positions in the mouse antibody. Such substitutions has been shown improve binding of humanized antibodies to the antigen in some instances. Intrabodies in which amino acids have been added, deleted, or substituted are referred to herein as modified intrabodies or altered intrabodies.

Nucleic Acid Molecules:

In some embodiments, the NFAT inhibitor can be a nucleic acid molecule, for example, a nucleic acid molecule that inhibits the expression of NFAT and/or CaN. In various embodiments, the nucleic acid molecule can be a DNA, RNA, siRNA, shRNA, or an artificial nucleic acid analog. Examples of an artificial nucleic acid include, but are not limited to, peptide nucleic acid, morpholino- and locked nucleic acid, as well as glycol nucleic acid and threose nucleic acid. Given the sequences encoding various NFAT family members and CaN disclosed in the art, a nucleic acid for use in the methods of the invention can be constructed using chemical synthesis and enzymatic ligation reactions using procedures known in the art. For example, a nucleic acid molecule can be chemically or recombinantly synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

In yet another embodiment, the NFAT inhibitory nucleic acid molecules can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup and Nielsen (1996) supra and Perry-O'Keefe et al. (1996) Proc. Natl. Acad. Sci. USA 93:14670-675.

Nucleic acid molecules can be produced by inserting the nucleic acid molecule into a vector and producing multiple copies of the vector and then isolating the nucleic acid sequence that encodes NFAT or CaN or a portion thereof.

In various embodiments, the structure of a NFAT antagonist can be modified for such purposes as enhancing therapeutic efficacy, or stability (e.g., ex vivo shelf life or resistance to proteolytic degradation in vivo). Modified NFAT antagonists can be produced, for instance, by amino acid substitution, deletion, or addition. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a NFAT antagonist results in a functional homolog can be readily determined by assessing the ability of the variant NFAT antagonistic peptide to produce a response (e.g., measurement of NFAT activation by luciferase reporter assay as described in the Examples) in neuronal cells in a fashion similar to the wild-type NFAT antagonistic peptide.

In some aspects of the invention, the NFAT antagonists can be cell-permeable, i.e., the NFAT antagonists can freely cross the cell membrane after contacting a cell. In some embodiments, to facilitate the delivery of the NFAT antagonist from the outside to the inside of neuronal cells, a vector can be used to express the NFAT antagonist into the neuronal cells. In general, as used herein, the term “vector” refers to any genetic element, such as a plasmid, phage, transposon, cosmid, chromosome, virus, virion, etc., that is capable of replication when associated with the proper control elements and that can carry gene sequences and express the respective product into cells. Thus, the term includes cloning and expression vehicles, as well as viral vectors.

In some embodiments, a viral vector can be used to express a NFAT antagonist, e.g., a peptide. Some viral-mediated expression methods employ retrovirus, adenovirus, lentivirus, herpes virus, pox virus, and adeno-associated virus (AAV) vectors, and such expression methods have been used in gene delivery and are well known in the art. For example, U.S. patent application No. 2002/0,193,335 provides methods of delivering a gene therapy vector, or transformed cell, to neurological tissue; U.S. patent application No. 2002/0,187,951 provides methods for treating or preventing a neurodegenerative disease in a mammal by administering a lentiviral vector to a target cell in the brain or nervous system of the mammal; U.S. patent application No. 2002/0,107,213 discloses a gene therapy vehicle and methods for its use in the treatment and prevention of neurodegenerative disease; U.S. patent application No. 2003/0,099,671 discloses a mutated rabies virus suitable for delivering a gene to a subject; and U.S. Pat. No. 6,310,196 describes a DNA construct which is useful for immunization or gene therapy; U.S. Pat. No. 6,436,708 discloses a gene delivery system which results in long-term expression throughout the brain has been developed; U.S. Pat. No. 6,140,111 which disclose retroviral vectors suitable for human gene therapy in the treatment of a variety of disease; and Kaspar B K et al. (2002) Mol Ther. 5:50-6, Suhr S T et al (1999) Arch Neurol. 56:287-92, Wong, P. C. et al. (2002) Nat Neurosci 5, 633-639) describes neuronal specific promoters such as Thy1 which can be employed in the methods of the invention.

Retroviral Gene Delivery:

Retroviruses provide a convenient platform for gene delivery. A selected gene can be inserted into a vector and packaged in retroviral particles using techniques known in the art. The recombinant virus can then be isolated and delivered to cells of the subject either in vivo or ex vivo. A number of retroviral systems have been described. See, e.g., U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Burns et al. (1993) Proc. Natl. Acad. Sci. USA 90:8033-37; Boris-Lawrie and Temin (1993) Curr. Opin. Genet. Develop. 3:102-09.

Replication-defective murine retroviral vectors are widely used gene transfer vectors. Murine leukemia retroviruses include a single stranded RNA molecule complexed with a nuclear core protein and polymerase (pol) enzymes, encased by a protein core (gag), and surrounded by a glycoprotein envelope (env) that determines host range. The genomic structure of retroviruses includes gag, pol, and env genes and 5′ and 3′ long terminal repeats (LTRs). Retroviral vector systems exploit the fact that a minimal vector containing the 5′ and 3′ LTRs and the packaging signal are sufficient to allow vector packaging, infection and integration into target cells, provided that the viral structural proteins are supplied in trans in the packaging cell line. Fundamental advantages of retroviral vectors for gene transfer include efficient infection and gene expression in most cell types, precise single copy vector integration into target cell chromosomal DNA and ease of manipulation of the retroviral genome.

Adenoviral Delivery:

In one embodiment of the invention, a nucleotide sequence encoding a NFAT antagonist is inserted into an adenovirus-based expression vector. Unlike retroviruses, which integrate into the host genome, adenoviruses persist extrachromosomally thus minimizing the risks associated with insertional mutagenesis (Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76).

The adenovirus genome is a linear double-stranded DNA molecule of approximately 36,000 base pairs with the 55-kDa terminal protein covalently bound to the 5′ terminus of each strand. Adenoviral (“Ad”) DNA contains identical Inverted Terminal Repeats (“ITRs”) of about 100 base pairs with the exact length depending on the serotype. The viral origins of replication are located within the ITRs exactly at the genome ends.

Adenoviral vectors have several advantages in gene therapy. They infect a wide variety of cells, have a broad host-range, exhibit high efficiencies of infectivity, direct expression of heterologous genes at high levels, and achieve long-term expression of those genes in vivo. The virus is fully infective as a cell-free virion so injection of producer cell lines is not necessary. With regard to safety, adenovirus is not associated with severe human pathology, and the recombinant vectors derived from the virus can be rendered replication defective by deletions in the early-region 1 (“E1”) of the viral genome. Adenovirus can also be produced in large quantities with relative ease. For all these reasons vectors derived from human adenoviruses, in which at least the E1 region has been deleted and replaced by a gene of interest, have been used extensively for gene therapy experiments in the pre-clinical and clinical phase.

Adenoviral vectors for use with the present invention can be derived from any of the various adenoviral serotypes, including, without limitation, any of the over 40 serotype strains of adenovirus, such as serotypes 2, 5, 12, 40, and 41. The adenoviral vectors used herein are replication-deficient and contain the gene of interest under the control of a suitable promoter, such as any of the promoters discussed below with reference to adeno-associated virus. For example, U.S. Pat. No. 6,048,551, incorporated herein by reference in its entirety, describes replication-deficient adenoviral vectors that can be used to include a NFAT antagonist under the control of the Rous Sarcoma Virus (RSV) promoter.

Other recombinant adenoviruses of various serotypes, and comprising different promoter systems, can be created by those skilled in the art. See, e.g., U.S. Pat. No. 6,306,652, incorporated herein by reference in its entirety.

Moreover, “minimal” adenovirus vectors as described in U.S. Pat. No. 6,306,652 will find use with the present invention. Such vectors retain at least a portion of the viral genome required for encapsidation (the encapsidation signal), as well as at least one copy of at least a functional part or a derivative of the ITR. Packaging of the minimal adenovirus vector can be achieved by co-infection with a helper virus or, alternatively, with a packaging-deficient replicating helper system.

Other useful adenovirus-based vectors for delivery of a NFAT antagonist include the “gutless” (helper-dependent) adenovirus in which the vast majority of the viral genome has been removed. Wu et al. (2001) Anesthes. 94:1119-32. Such “gutless” adenoviral vectors produce essentially no viral proteins, thus allowing gene therapy to persist for over a year after a single administration. Parks (2000) Clin. Genet. 58:1-11; Tsai et al. (2000) Curr. Opin. Mol. Ther. 2:515-23. In addition, removal of the viral genome creates space that can be used to insert control sequences that provide for regulation of transgene expression by systemically administered drugs (Burcin et al. (1999) Proc. Natl. Acad. Sci. USA 96:355-60), adding both safety and control of virally driven protein expression. These and other recombinant adenoviruses will find use with the present methods.

Adeno Associated Virus (AAV):

One viral system that has been used for gene delivery is AAV. AAV is a parvovirus which belongs to the genus Dependovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions, facilitating production, storage and transportation.

The AAV genome is a linear single-stranded DNA molecule containing approximately 4681 nucleotides. The AAV genome generally comprises an internal non-repeating genome flanked on each end by inverted terminal repeats (ITRs). The ITRs are approximately 145 base pairs (bp) in length. The ITRs have multiple functions, including serving as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins is expressed from the AAV rep region, Rep 78, Rep 68, Rep 52, and Rep 40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus or vaccinia) in order to form AAV virions in the wild. In the absence of co-infection with a helper virus, AAV establishes a latent state in which the viral genome inserts into a host cell chromosome, but infectious virions are not produced. Subsequent infection by a helper virus rescues the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

Adeno-associated virus (AAV) has been used with success in gene therapy. AAV has been engineered to deliver genes of interest by deleting the internal nonrepeating portion of the AAV genome (i.e., the rep and cap genes) and inserting a heterologous gene (in this case, the gene encoding the anti-inflammatory cytokine) between the ITRs. The heterologous gene is typically functionally linked to a heterologous promoter (constitutive, cell-specific, or inducible) capable of driving gene expression in the patient's target cells under appropriate conditions.

Recombinant AAV virions comprising a NFAT antagonistic nucleic acid or peptide/protein can be produced using a variety of art-recognized techniques. In one embodiment, a rAAV vector construct is packaged into rAAV virions in cells co-transfected with wild-type AAV and a helper virus, such as adenovirus. See, e.g., U.S. Pat. No. 5,139,941.

Alternatively, plasmids can be used to supply the necessary replicative functions from AAV and/or a helper virus. In one embodiment of the present invention, rAAV virions are produced using a plasmid to supply necessary AAV replicative functions (the “AAV helper functions”). See e.g., U.S. Pat. Nos. 5,622,856 and 5,139,941, both incorporated herein by reference in their entireties. In another embodiment, a triple transfection method is used to produce rAAV virions. The triple transfection method is described in detail in U.S. Pat. Nos. 6,001,650 and 6,004,797, which are incorporated by reference herein in their entireties. The triple transduction method is advantageous because it does not require the use of an infectious helper virus during rAAV production, enabling production of a stock of rAAV virions essentially free of contaminating helper virus. This is accomplished by use of three vectors for rAAV virion production: an AAV helper function vector, an accessory function vector, and a rAAV expression vector. One of skill in the art will appreciate, however, that the nucleic acid sequences encoded by these vectors can be provided on two or more vectors in various combinations. Vectors and cell lines necessary for preparing helper virus-free rAAV stocks are commercially available as the AAV Helper-Free System (Catalog No. 240071) (Stratagene, La Jolla, Calif.).

The AAV helper function vector encodes AAV helper function sequences (i.e., rep and cap) that function in trans for productive rAAV replication and encapsidation. Preferably, the AAV helper function vector supports efficient rAAV virion production without generating any detectable replication competent AAV virions (i.e., AAV virions containing functional rep and cap genes). An example of such a vector, pHLP19, is described in U.S. Pat. No. 6,001,650. The rep and cap genes of the AAV helper function vector can be derived from any of the known AAV serotypes. For example, the AAV helper function vector may have a rep gene derived from AAV-2 and a cap gene derived from AAV-6. One of skill in the art will recognize that other rep and cap gene combinations are possible, the defining feature being the ability to support rAAV virion production.

The accessory function vector encodes nucleotide sequences for non-AAV-derived viral and/or cellular functions upon which AAV is dependent for replication (the “accessory functions”). The accessory functions include those functions required for AAV replication, including, without limitation, genes involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In one embodiment, the accessory function plasmid pLadeno5 can be used. See U.S. Pat. No. 6,004,797. This plasmid provides a complete set of adenovirus accessory functions for AAV vector production, but lacks the components necessary to form replication-competent adenovirus.

Unlike stocks of rAAV vectors prepared using infectious helper virus, stocks prepared using an accessory function vector (e.g. the triple transfection method) do not contain contaminating helper virus because no helper virus is added during rAAV production. Even after purification, for example by CsCl density gradient centrifugation, rAAV stocks prepared using helper virus still remain contaminated with some level of residual helper virus. When adenovirus is used as the helper virus in preparing a stock of rAAV virions, contaminating adenovirus can be inactivated by heating to temperatures of approximately 60° C. for 20 minutes or more. This treatment effectively inactivates only the helper virus since AAV is extremely heat stable, while the helper adenovirus is heat labile. Although heat inactivating of rAAV stocks may render much of the contaminating adenovirus non-infectious, it does not physically remove the helper virus proteins from the stock. Such contaminating viral protein can elicit undesired immune responses in subjects and are to be avoided if possible. Contaminating adenovirus particles and proteins in rAAV stocks can be avoided by use of the accessory function vectors disclosed herein.

Recombinant AAV Expression Vectors:

Recombinant AAV expression vectors can be constructed using standard techniques of molecular biology. rAAV vectors comprise a transgene of interest (e.g. a sequence comprising VIVIT (SEQ ID NO: 7)) flanked by AAV ITRs at both ends. rAAV vectors are also constructed to contain transcription control elements operably linked to the transgene sequence, including a transcriptional initiation region and a transcriptional termination region. The control elements are selected to be functional in a mammalian target cell.

The nucleotide sequences of AAV ITR regions are known. See, e.g., Kotin (1994) Human Gene Therapy 5:793-801; Berns “Parvoviridae and their Replication” in Fundamental Virology, 2nd Edition, (B. N. Fields and D. M. Knipe, eds.) for the AAV-2 sequence. AAV ITRs used in the vectors of the invention need not have a wild-type nucleotide sequence, and may be altered, e.g., by the insertion, deletion or substitution of nucleotides. Additionally, AAV ITRs may be derived from any of several AAV serotypes, including without limitation, AAV-1, AAV-2, AAV-3, AAV-4, AAV-5, AAV-6, AAV-7 and AAV-8, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

Suitable transgenes for delivery in AAV vectors can be less than about 5 kilobases (kb) in size. In one embodiment, a NFAT antagonist, e.g., a peptide comprising an amino acid sequence of VIVIT (SEQ ID NO: 7), can be delivered with AAV vectors. In other embodiments, a DNA sequence encoding a VIVIT peptide (SEQ ID NO: 7), and optionally a nuclear localization sequence (NLS) can be delivered with AAV vectors. The selected polynucleotide sequence is operably linked to control elements that direct the transcription thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, neuron-specific enolase promoter, a GFAP promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.).

The AAV expression vector harboring a transgene of interest bounded by AAV ITRs can be constructed by directly inserting the selected sequence(s) into an AAV genome that has had the major AAV open reading frames (“ORFs”) excised. Other portions of the AAV genome can also be deleted, so long as enough of the ITRs remain to provide replication and packaging functions. Such constructs can be designed using techniques well known in the art. See, e.g., U.S. Pat. Nos. 5,173,414 and 5,139,941; International Publication Nos. WO 92/01070 and WO 93/03769; Lebkowski et al. (1988) Molec. Cell. Biol. 8:3988-96; Vincent et al. (1990) Vaccines 90 (Cold Spring Harbor Laboratory Press); Carter (1992) Current Opinion in Biotechnology 3:533-39; Muzyczka (1992) Current Topics in Microbiol. and Immunol. 158:97-129; Kotin (1994) Human Gene Therapy 5:793-801; Shelling and Smith (1994) Gene Therapy 1:165-69; and Zhou et al. (1994) J. Exp. Med. 179:1867-75.

AAV ITR-containing DNA fragments can be ligated at both ends of a selected transgene using standard techniques, such as those described in Sambrook et al., supra. For example, ligations can be accomplished in 20 mM Tris-Cl pH 7.5, 10 mM MgCl2, 10 mM DTT, 33 μg/ml BSA, 10 mM-50 mM NaCl, and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration).

Suitable host cells for producing rAAV virions of the present invention from rAAV expression vectors include microorganisms, yeast cells, insect cells, and mammalian cells. Such host cells are preferably capable of growth in suspension culture, a bioreactor, or the like. The term “host cell” includes the progeny of the original cell that has been transfected with an rAAV virion. Cells from the stable human cell line, 293 (readily available through the American Type Culture Collection under Accession Number ATCC CRL1573) are preferred in the practice of the present invention. The human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a particularly convenient platform in which to produce rAAV virions.

Other Viral Vectors for Delivery:

Additional viral vectors useful for delivering the nucleic acid molecules and/or expressing a NFAT antagonist include those derived from the pox family of viruses, including vaccinia virus and avian poxvirus. By way of example, vaccinia virus recombinants expressing a NFAT antagonist can be constructed as follows. DNA carrying the NFAT antagonist is inserted into an appropriate vector adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter and the gene into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can be used to express a NFAT antagonist. Recombinant avipox viruses expressing immunogens from mammalian pathogens are known to confer protective immunity when administered to non-avian species. The use of avipox vectors in human and other mammalian species is advantageous with regard to safety because members of the avipox genus can only productively replicate in susceptible avian species. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors, can also be used for gene delivery. Michael et al. (1993) J. Biol. Chem. 268:6866-69 and Wagner et al. (1992) Proc. Natl. Acad. Sci. USA 89:6099-6103. Members of the Alphavirus genus, for example the Sindbis and Semliki Forest viruses, may also be used as viral vectors for delivering and expressing a NFAT antagonist. See, e.g., Dubensky et al. (1996) J. Virol. 70:508-19; WO 95/07995; WO 96/17072.

### Effective Amount of a NFAT Antagonist

In methods of the invention, a population of neuronal cells is contacted with an effective amount of at least one NFAT antagonist. The phrase “effective amount” as used herein refers to an amount of a compound, material, or composition which is effective for producing some desired effect in at least a sub-population of cells. For example, a population of neuronal cells is contacted with an amount of a NFAT antagonist described herein sufficient to produce a statistically significant, measurable response as described in Examples 6 and 8, when compared to neuronal cells in the absence of a NFAT antagonist. In some embodiments, the effective amount is sufficient to decrease NFAT activity of one or more neuronal cells by at least about 5%, e.g., by at least about 10%, at least about 20%, at least about 30%, at least about 40%, at least about 50%, at least a bout 60%, at least about 70%, at least about 80%, at least about 90%, at least about 95%, at least about 96%, about 98%, about 99%, or 100%, as compared to neuronal cells in the absence of the NFAT antagonist. As used herein, the term “NFAT activity” refers to level of NFAT dephosphorylation and/or nuclear translocation of NFAT. In some embodiments, the NFAT activity refers to calcineurin-mediated NFAT activity, i.e., binding of calcineurin (CaN) to NFAT, which in turns activates the transcriptional factor NFAT and its nuclear translocation. In vitro determination of NFAT dephosphorylation level and nuclear translocation of NFAT are well recognized by a skilled artisan, e.g., by western blot analysis, luciferase reporter assay and/or immunohistochemistry as described in the Examples. NFAT activity can also be determined by measuring transcriptional activation of genes, e.g., using a vector comprising a reporter, and an NFAT promoter.

In some embodiments, the effective amount is sufficient to reduce neuodengerative morphologies that occur in neuronal cells. Various established in vitro and in vivo assays can be used to determine an effective amount of the NFAT antagonist for inhibiting neurodegeneration in neuronal cells. For example, multiphoton imaging enables quantitatively determination of morphological changes associated with neurodegeneration, e.g., neuritic dystrophies, neurite curvature, and as spine density, in a living subject, e.g., a mouse, as described in the Examples. Exemplary measurable responses are dendritic spine density and neuritic dystrophies, which can be determined by immunochemistry for in vitro characterization or by multiphoton imaging for in vivo characterization as described herein. In accordance with aspects of the invention, treatment of neuronal cells with an effective amount of a NFAT antagonist, e.g., a VIVIT peptide (SEQ ID NO: 7), reduces such Aβ-associated neurodegenerative alterations in vivo. Thus, enhanced dendritic spine density and/or reduced neuritic dystrophies observed in treated neuronal cells can be indicative of treatment efficacy.

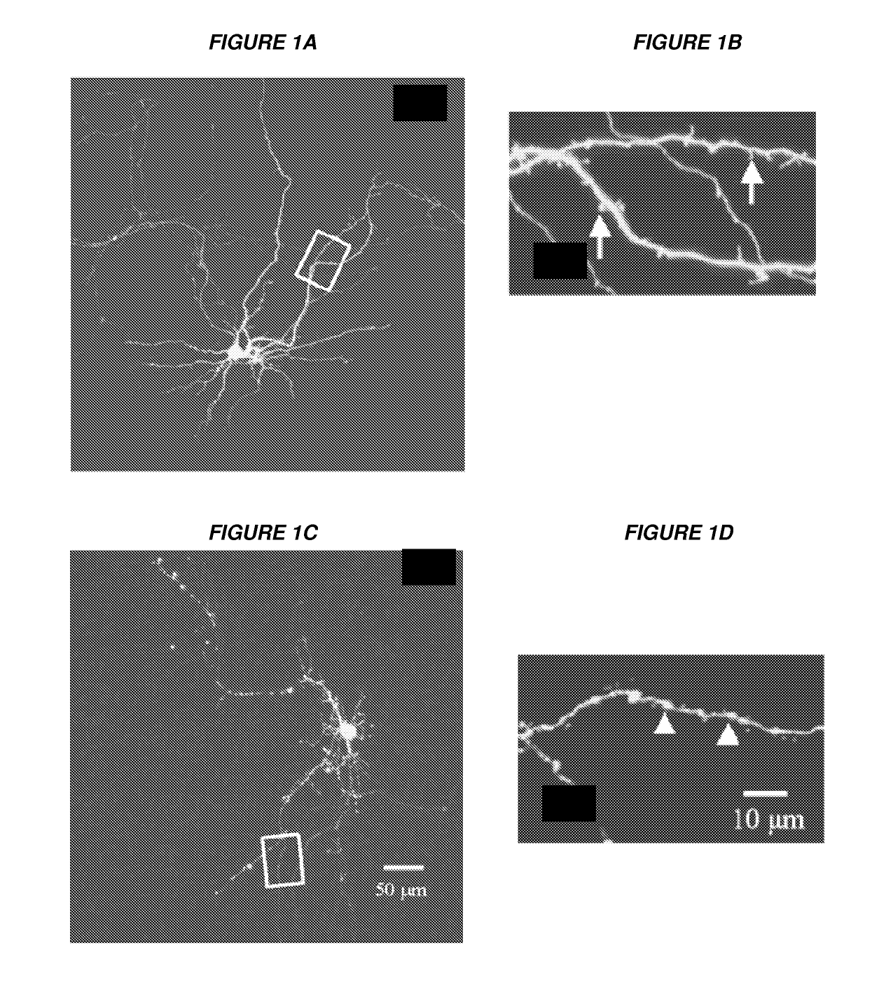

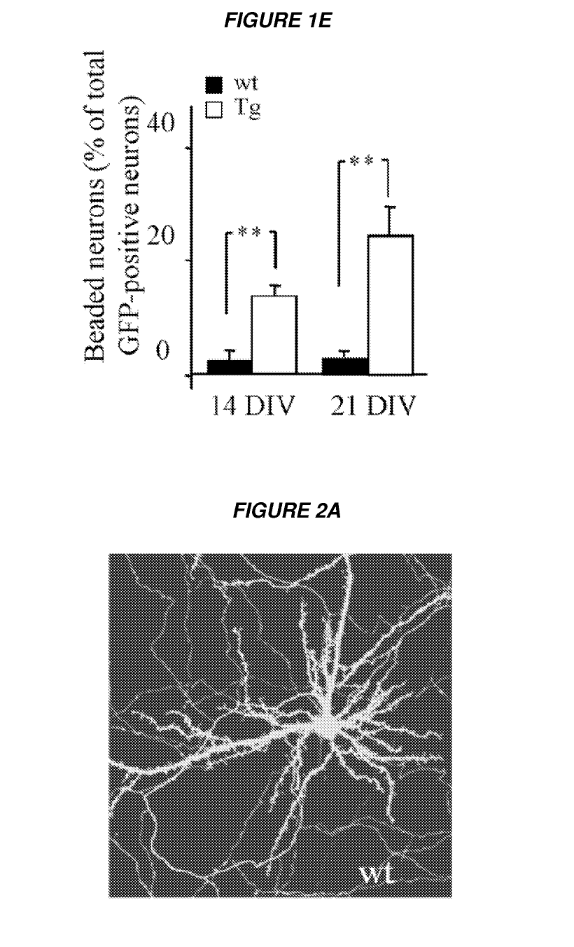

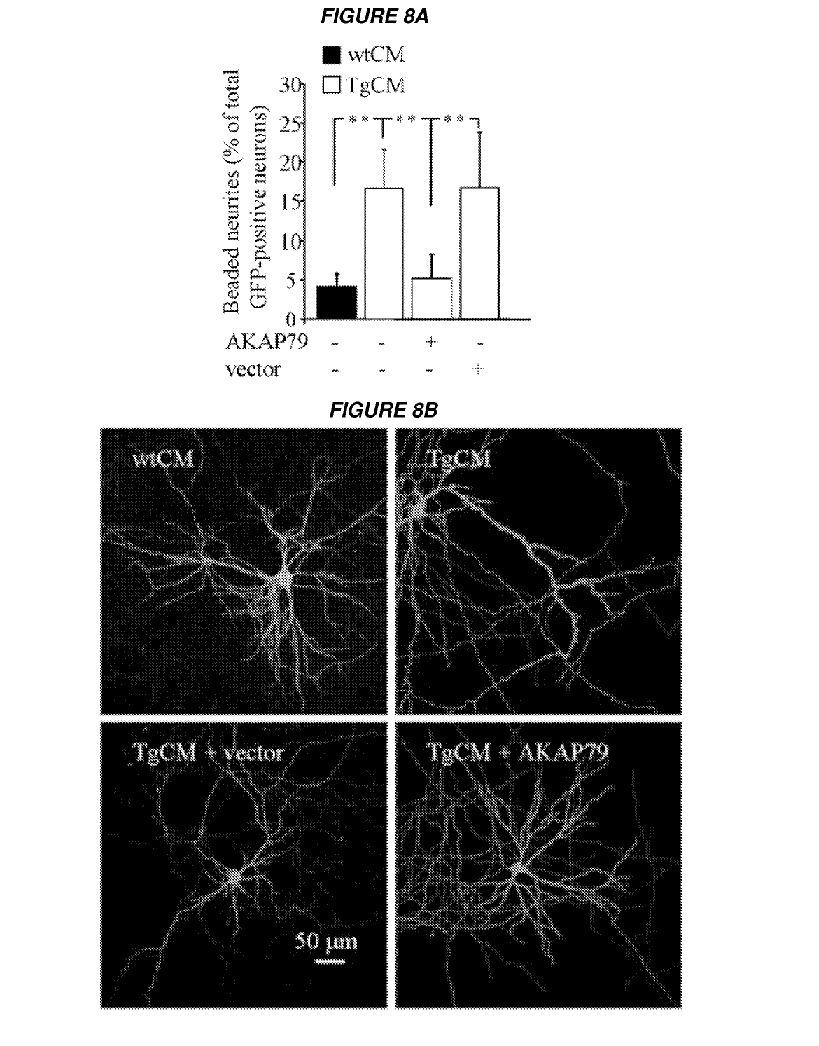

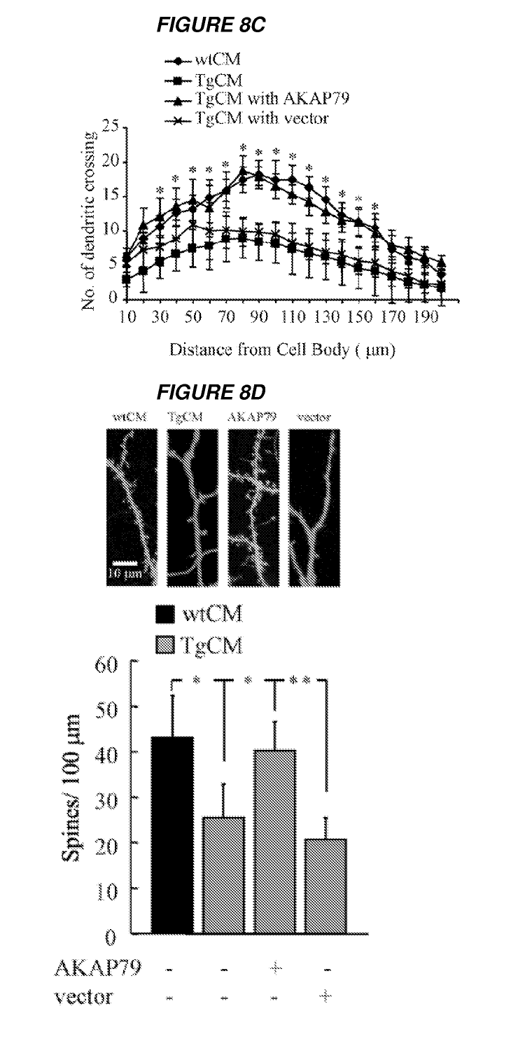

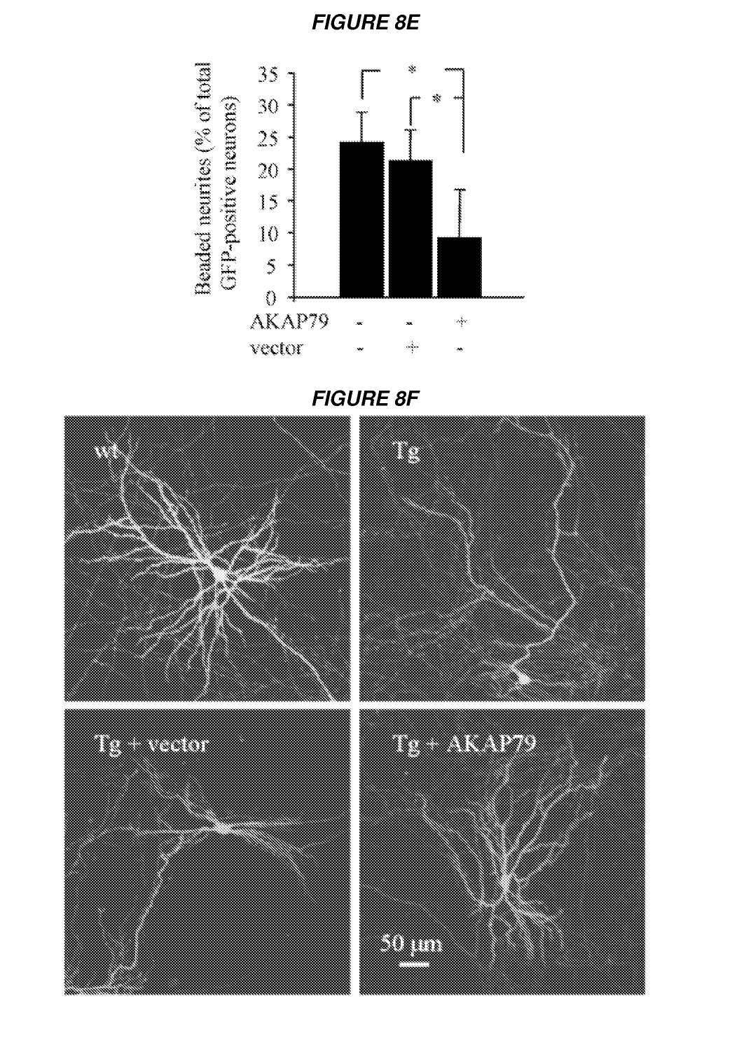

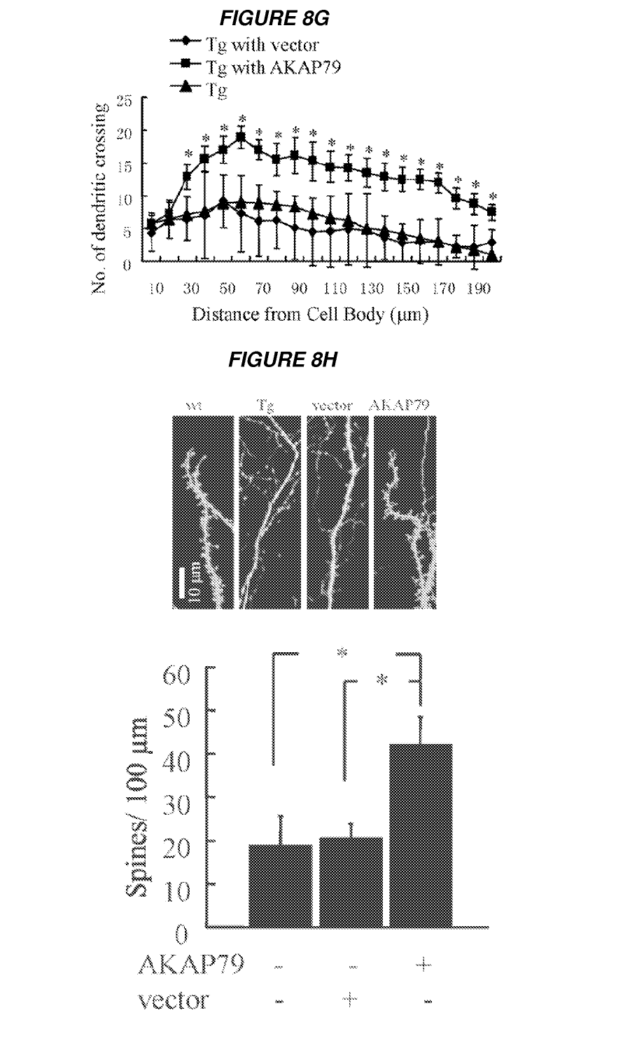

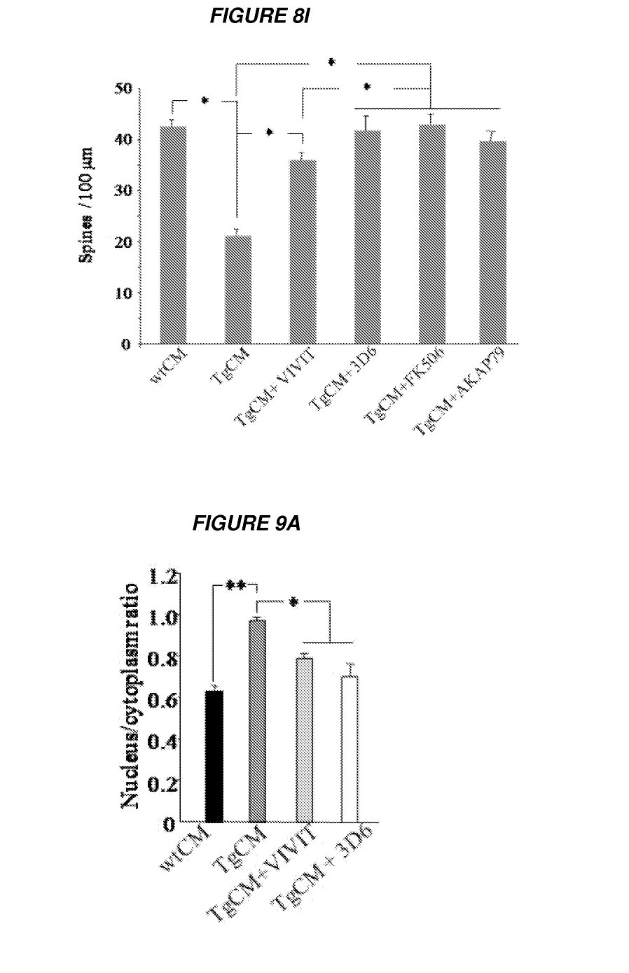

Accordingly, in some embodiments, the effective amount of a NFAT antagonist is sufficient to decrease neuritic dystrophies of one or more neuronal cells by at least about 5%, e.g., by at least about 10%, at least about 20%, at least about 30%, at least about 40%, at least about 50%, at least a bout 60%, at least about 70%, at least about 80%, at least about 90%, at least about 95%, at least about 96%, about 98%, about 99%, or 100%, as compared to neuronal cells in the absence of the NFAT antagonist. As used herein, the term “neuritic dystrophy” means a distortion of the shapes of neurites (that is, axons and dendrites), which can be visualized microscopically in the brains of a subject. A skilled practitioner, e.g., a pathologist, can readily identify neurodegenerative morphologies. As shown in FIGS. 1A to 1D, a wild-type neuronal cell displays branched dendritic arbors studded with protrusions that include dendritic spines and filopodia, while a neurodegenerative neuronal cell exhibits focal neuritic swellings. As demonstrated in the Examples (see FIGS. 8E to 8F, FIGS. 9F to 9G and FIG. 12A), neuritic dystrophy can be determined microscopically in vitro or in vivo by areas of swelling in a neurons's dendrite.

In some embodiments, the effective amount of a NFAT antagonist is sufficient to decrease increase dendritic spine density of one or more neuronal cells by at least about 5%, e.g., by at least about 10%, at least about 20%, at least about 30%, at least about 40%, at least about 50%, at least a bout 60%, at least about 70%, at least about 80%, at least about 90%, at least about 95%, at least about 96%, about 98%, about 99%, or 100%, as compared to neuronal cells in the absence of the NFAT antagonist. Dendritic spine density is a measurement of the number of small membranous protrusions from a neuron's dendrite. It can be microscopically determined in vitro or in vivo, as shown in FIGS. 8H, 9I and 12B.

In some embodiments, the effective amount of a NFAT antagonist is about 0.1 mg/kg to about 100 mg/kg. In some embodiments, the effective amount of a NFAT antagonist can be present in an amount of about 0.5 mg/kg to about 100 mg/kg, about 1 mg/kg to about 75 mg/kg, about 3 mg/kg to about 50 mg/kg, about 5 mg/kg to about 25 mg/kg, or about 5 mg/kg to about 15 mg/kg. In some embodiments, the effective amount of a NFAT antagonist is about 10 mg/kg.

In some embodiments, the effective amount of a NFAT antagonist is about 0.01 μM to about 100 μM. In some embodiments, the effective amount of a NFAT antagonist can be present in an amount of about 0.05 μM to about 50 μM, about 0.05 μM to about 25 μM, about 0.05 μM to about 10 μM, about 0.05 μM to about 5 μM, or about 1 μM to about 3 μM. In one embodiment, the effective amount of a NFAT antagonist, e.g., a VIVIT-containing peptide, is about 2 μM.

Compositions of the Invention

Another aspect of the invention encompasses compositions comprising an effective amount of at least one NFAT antagonist described herein. In one embodiment, the composition further comprises at least one additional agent that inhibits neurodegeneration, e.g., a AKAP79 peptide, or FK506.

Since methods of the invention can be used in vitro, in one embodiment, the composition further comprises a cell culture medium. As used herein, the term “cell culture medium” refers to any nutrient medium in which cardiac stem cells can be cultured in vitro. Examples of nutrients essential to cell metabolism and proliferation, e.g., amino acids, lipids, carbohydrates, vitamins and mineral salts can be included in the cell culture medium. In one embodiment, cell culture medium also comprises any substance essential to cell differentiation. One of skill in the art can determine an appropriate formulation of cell culture medium for culturing neuronal cells, based on the cell condition (e.g., morphology, viability, growth rate and cell density).

In some embodiments, a vector can be used to express and deliver the NFAT antagonist into the cells. For example, a viral vector as described herein with an expression cassette can encode a NFAT antagonist sequence. In such embodiments, the composition of the invention can comprise a concentration of viral vectors from about 10⁴ viral genomes/ml to about 10²⁰ viral genomes/ml, from about 10⁵ viral genomes/ml to about 10¹⁸ viral genomes/ml, from about 10⁶ viral genomes/ml to about 10¹⁵ viral genomes/ml, or from about 10¹⁰ viral genomes/ml to about 10¹⁵ viral genomes/ml. In some embodiments, the composition can comprise a concentration of viral vectors from about 1×10¹² viral genomes/ml to about 1×10¹³ viral genomes/ml. Depending on the use of compositions of the invention, a skilled artisan can determine an appropriate concentration of the viral vectors in a composition. For example, for cell culture compositions, e.g., comprising a cell culture medium, lower concentrations of viral vectors, e.g., 1×10⁵ viral genomes/ml-1×10⁸ viral genomes/ml can be selected for a culturing purpose. For therapeutic administration purpose, the composition of the invention can comprise higher concentrations of viral vectors, e.g., about 1×10¹⁰ viral genomes/ml to about 1×10¹⁵ viral genomes/ml. The precise determination of an effective dose can be based on individual factors, including their plaque size, age, and amount of time since neurodegeneration. Therefore, dosages can be readily adjusted for each individual patient by those skilled in the art.

For administration to a subject in need thereof, e.g., a subject diagnosed with or predisposed to Alzheimer's disease, the NFAT antagonist and/or viral expression vector encoding the NFAT antagonist can be provided in a pharmaceutically acceptable composition. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further comprise one or more pharmaceutically carriers (additives) and/or diluents. As used herein, the term “pharmaceutically-acceptable carrier” means a pharmaceutically-acceptable material, composition or vehicle, such as a liquid, diluent, excipient, manufacturing aid or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include, but are not limited to, gelatin, buffering agents, such as magnesium hydroxide and aluminum hydroxide, pyrogen-free water, isotonic saline, Ringer's solution, pH buffered solutions, bulking agents such as polypeptides and amino acids, serum component such as serum albumin, HDL and LDL, and other non-toxic compatible substances employed in pharmaceutical formulations. Preservatives and antioxidants can also be present in the formulation. The terms such as “excipient”, “carrier”, “pharmaceutically acceptable carrier” or the like are used interchangeably herein.

Pharmaceutically acceptable carriers can vary in a composition of the invention, depending on the administration route and formulation. For example, the pharmaceutically acceptable composition of the invention can be delivered via injection. These routes for administration (delivery) include, but are not limited to, subcutaneous or parenteral including intravenous, intracortical, intracranial, intramuscular, intraperitoneal, and infusion techniques. In one embodiment, the pharmaceutical acceptable composition is in a form that is suitable for intracortical injection. In another embodiment, the pharmaceutical composition is formulated for intracranial injection. Other forms of administration can be also be employed, e.g., oral, systemic, or parenteral administration.

The NFAT antagonist and/or the composition thereof can be formulated in pharmaceutically acceptable compositions which comprise a therapeutically-effective amount of the compound, formulated together with one or more pharmaceutically acceptable carriers (additives) and/or diluents. The compounds can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

When administering a pharmaceutical composition of the invention parenterally, it will be generally formulated in a unit dosage injectable form (solution, suspension, emulsion). The pharmaceutical formulations suitable for injection include sterile aqueous solutions or dispersions. The carrier can be a solvent or dispersing medium containing, for example, water, cell culture medium, buffers (e.g., phosphate buffered saline), polyol (for example, glycerol, propylene glycol, liquid polyethylene glycol, and the like), and suitable mixtures thereof. In some embodiments, the pharmaceutical carrier can be a buffered solution (e.g. PBS).

In some embodiments, the pharmaceutical composition can be formulated in an emulsion or a gel. In such embodiments, at least one NFAT antagonist or viral vector encoding a NFAT antagonist can be encapsulated within a biocompatible gel, e.g., hydrogel and a peptide gel. The gel pharmaceutical composition can be implanted to the brain near the degenerating neuronal cells, e.g., the cells in proximity to the amyloid plaque.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation. With respect to compositions of the invention, however, any vehicle, diluent, or additive used should have to be biocompatible or inert with the NFAT antagonist or a vector encoding the NFAT antagonist.

The compositions can be isotonic, i.e., they can have the same osmotic pressure as blood and lacrimal fluid. The desired isotonicity of the compositions of the invention can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions.

Viscosity of the compositions can be maintained at the selected level using a pharmaceutically acceptable thickening agent. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

In some embodiment, the neuronal cells transduced with a vector encoding a NFAT antagonist can be included in the compositions of the invention and stored frozen. In such embodiments, an additive or preservative known for freezing cells can be included in the compositions. A suitable concentration of the preservative can vary from 0.02% to 2% based on the total weight although there may be appreciable variation depending upon the preservative or additive selected. One example of such additive or preservative can be dimethyl sulfoxide (DMSO) or any other cell-freezing agent known to a skilled artisan. In such embodiments, the composition will be thawed before use or administration to a subject, e.g., neuronal stem cell therapy.

Typically, any additives (in addition to the active NFAT antagonist) can be present in an amount of 0.001 to 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. For any therapeutic composition to be administered to a subject in need thereof, and for any particular method of administration, it is preferred to determine toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan.

The compositions of the invention can be prepared by mixing the ingredients following generally-accepted procedures. For example, an effective amount of a NFAT antagonist or vectors encoding a NFAT antagonist can be re-suspended in an appropriate pharmaceutically acceptable carrier and the mixture can be adjusted to the final concentration and viscosity by the addition of water or thickening agent and possibly a buffer to control, pH or an additional solute to control tonicity. An effective amount of at least one a NFAT antagonist described herein and any other additional agent, e.g., for inhibiting neurodegeneration, can be mixed with the cell mixture. Generally the pH can vary from about 3 to about 7.5. In some embodiments, the pH of the composition can be about 6.5 to about 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by a skilled artisan.

Suitable regimes for initial administration and further doses or for sequential administrations can be varied. In one embodiment, a therapeutic regimen includes an initial administration followed by subsequent administrations, if necessary. In some embodiments, multiple administrations of a NFAT antagonist can be injected to the subject's brain. For example, a NFAT antagonist can be administered in two or more, three or more, four or more, five or more, or six or more injections. In some embodiments, the same NFAT antagonist can be administered in each subsequent administration. In some embodiments, a different NFAT antagonist described herein can be administered in each subsequent administration. Injections can be made in cortex, e.g., somatosensory cortex. In other embodiments, injections can be administered in proximity to a plaque, e.g., amyloid-beta plaque.

The subsequent injection can be administered immediately after the previous injection, or after at least about 1 minute, after at least about 2 minute, at least about 5 minutes, at least about 15 minutes, at least about 30 minutes, at least about 1 hour, at least about 2 hours, at least about 3 hours, at least about 6 hours, at least about 12 hours, at least about 24 hours, at least about 2 days, at least about 3 days, at least about 4 days, at least about 5 days, at least about 6 days or at least about 7 days. In some embodiments, the subsequent injection can be administered after at least about 1 week, at least about 2 weeks, at least about 1 month, at least about 2 years, at least about 3 years, at least about 6 years, or at least about 10 years.

In various embodiments, a dosage comprising a composition of the invention is considered to be pharmaceutically effective if the dosage reduce degree of neurodegeneration, e.g., indicated by changes in neurodegenerative morphologies or improvement in brain or cognitive function, by at least about 5%, at least about 10%, at least about 15%, at least about 20%, at least about 30%, at least about 40%, or at least about 50%. In one embodiment, the brain or cognitive function is improved by more than 50%, e.g., at least about 60%, or at least about 70%. In another embodiment, the brain or cognitive function is improved by at least about 80%, at least about 90% or greater, as compared to a control (e.g. in the absence of the composition described herein).

Selection of Subjects in Need Thereof.

Yet another aspect of the invention relates to the use of methods and compositions described herein to treat Alzheimer's disease (AD) in a subject in need thereof. The inventors have demonstrated increased levels of NFATc4 in the nuclear fraction from the cortex of patients with AD, and the neurodegenerative morphologies in a mouse model of AD can be ameliorated by NFAT inhibition, as compared to in the absence of the NFAT antagonist. Accordingly, the method of treating AD in a subject in need thereof, comprising contacting a population of neuronal cells in the subject with an effective amount of a NFAT antagonist.

The terms “treatment” and “treating” as used herein, with respect to treatment of a disease, means preventing the progression of the disease, or altering the course of the disorder (for example, but are not limited to, slowing the progression of the disorder), or reversing a symptom of the disorder or reducing one or more symptoms and/or one or more biochemical markers in a subject, preventing one or more symptoms from worsening or progressing, promoting recovery or improving prognosis. For example, in the case of treating a neurodegenerative disorder, e.g., AD, therapeutic treatment refers to reduced neurodegenerative morphologies, e.g., reduced neurite dystrophies described herein after administration of the composition of the invention. In another embodiment, the therapeutic treatment refers to alleviation of at least one symptom associated with a neurodegenerative disease, e.g., AD. Measurable lessening includes any statistically significant decline in a measurable marker or symptom, such as assessing the cognitive improvement with neuropsychological tests such as verbal and perception after treatment. In one embodiment, at least one symptom of a neurodegenerative disorder, e.g., AD, is alleviated by at least about 10%, at least about 15%, at least about 20%, at least about 30%, at least about 40%, or at least about 50%. In another embodiment, at least one symptom is alleviated by more than 50%, e.g., at least about 60%, or at least about 70%. In one embodiment, at least one symptom is alleviated by at least about 80%, at least about 90% or greater, as compared to a control (e.g. in the absence of the composition described herein).

In some embodiments, the method of treatment further comprises a step of diagnosing a subject with AD prior to the contacting. Subjects amenable to methods of treatment are subjects that have been diagnosed with Alzheimer's disease. Methods for diagnosing Alzheimer's disease are well known in the art. For example, the stage of Alzheimer's disease can be assessed using the Functional Assessment Staging (FAST) scale, which divides the progression of Alzheimer's disease into 16 successive stages under 7 major headings of functional abilities and losses: Stage 1 is defined as a normal adult with no decline in function or memory. Stage 2 is defined as a normal older adult who has some personal awareness of functional decline, typically complaining of memory deficit and forgetting the names of familiar people and places. Stage 3 (early Alzheimer's disease) manifests symptoms in demanding job situation, and is characterized by disorientation when traveling to an unfamiliar location; reports by colleagues of decreased performance; name- and word-finding deficits; reduced ability to recall information from a passage in a book or to remember a name of a person newly introduced to them; misplacing of valuable objects; decreased concentration. In stage 4 (mild Alzheimer's Disease), the patient may require assistance in complicated tasks such as planning a party or handling finances, exhibits problems remembering life events, and has difficulty concentrating and traveling. In stage 5 (moderate Alzheimer's disease), the patient requires assistance to perform everyday tasks such as choosing proper attire. Disorientation in time, and inability to recall important information of their current lives, occur, but patient can still remember major information about themselves, their family and others. In stage 6 (moderately severe Alzheimer's disease), the patient begins to forget significant amounts of information about themselves and their surroundings and require assistance dressing, bathing, and toileting. Urinary incontinence and disturbed patterns of sleep occur. Personality and emotional changes become quite apparent, and cognitive abulia is observed. In stage 7 (severe Alzheimer's disease), speech ability becomes limited to just a few words and intelligible vocabulary may be limited to a single word. A patient can lose the ability to walk, sit up, or smile, and eventually cannot hold up the head.

Other alternative diagnostic methods for AD include, but not limited to, cellular and molecular testing methods disclosed in US Patent No.: U.S. Pat. No. 7,771,937, U.S. Pat. No. 7,595,167, US 55580748, and PCT Application No.: WO2009/009457, the content of which is incorporated by reference in its entirety. Additionally, protein-based biomarkers for AD, some of which can be detected by non-invasive imaging, e.g., PET, are disclosed in U.S. Pat. No. 7,794,948, the content of which is incorporated by reference in its entirety.

Genes involved in AD risk can be used for diagnosis of AD. One example of other AD risk genes is apolipoprotein E-ε4 (APOE-ε4). APOE-ε4 is one of three common forms, or alleles, of the APOE gene; the others are APOE-e2 and APOE-e3. APOE provides the blueprint for one of the proteins that carries cholesterol in the bloodstream. Everyone inherits a copy of some form of APOE from each parent. Those who inherit one copy of APOE-ε4 have an increased risk of developing AD. Those who inherit two copies have an even higher risk, but not a certainty of developing AD. In addition to raising risk, APOE-ε4 may tend to make symptoms appear at a younger age than usual. Other AD risk genes in addition to APOE-e4 are well established in the art. Some of them are disclosed in US Pat. App. No.: US 2010/0249107, US 2008/0318220, US 2003/0170678 and PCT Application No.: WO 2010/048497, the content of which is incorporated by reference in its entirety. Genetic tests are well established in the art and are available, for example for APOE-e4. A subject carrying the APOE-ε4 allele can, therefore, be identified as a subject at risk of developing AD.

In further embodiments, subjects with Aβ burden are amenable to the methods described herein. Such subjects include, but not limited to, the ones with Down syndrome, Huntington disease, the unaffected carriers of APP or presenilin gene mutations, and the late onset AD risk factor, apolipoprotein E-ε4.

In some embodiments, AD patients that are currently receiving other AD therapeutic treatment can also be subjected to the methods of treatment as described herein.

In some embodiments, a subject who has been diagnosed with an increased risk for developing AD, e.g., using the diagnostic methods and assays described herein or any AD diagnostic methods known in the art, can be subjected to the methods of treatment as described herein.

In some embodiments, the subject selected for the methods described herein can be previously diagnosed with AD and is being under a treatment regimen.

As used herein, a “subject” can mean a human or an animal. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. A Patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female.

In one embodiment, the subject is a mammal. The mammal can be a human, non-human primate, mouse, rat, dog, cat, horse, or cow, but are not limited to these examples. Mammals other than humans can be advantageously used as subjects that represent animal models of stem cell therapy for repair for damaged myocardium. In addition, the methods and compositions described herein can be employed in domesticated animals and/or pets.

Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the paragraphed invention, because the scope of the invention is limited only by the paragraphs. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. Thus for example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

As used herein, the term “administer” or “administration” refers to the placement of a composition into a subject by a method or route which results in at least partial localization of the composition at a desired site such that desired effect is produced. Routes of administration suitable for the methods of the invention include, but are not limited to, injection. Generally, local administration results in more of the composition being delivered to a specific location as compared to the entire body of the subject. In some embodiments, the terms “administration”, “contacting”, and “treating” in relative to exposing neuronal cells to a NFAT antagonist are used interchangeably.

The term “hydrogel” as used herein refers to natural or synthetic polymers that show superabsorbent properties (having even over 99% water) and possess a degree of flexibility similar to natural tissue, due to their significant water content. Examples of hydrogels used as scaffolds in tissue engineering or reservoirs in local drug delivery include, but are not limited to, methylcellulose, hylaronan, and other naturally derived polymers. In one embodiment, the hydrogel is biodegradable.

The term “increase” or “enhance” as used herein generally means an increase by a statistically significant amount. In one embodiment, “increase” or “enhance” refers to an increase by at least 10% as compared to a reference level, for example an increase by at least about 20%, or at least about 30%, or at least about 40%, or at least about 50%, or at least about 60%, or at least about 70%, or at least about 80%, or at least about 90% or up to and including a 100% increase, or any increase between 10-100% as compared to a reference level. The reference level as used herein refers to a control in the absence of, e.g., a NFAT antagonist. In one embodiment, the reference level is measured prior to administration of the composition described herein.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

The present invention may be defined in any of the following numbered paragraphs:
1. A method of treating Alzheimer's disease (AD) in a subject in need thereof, comprising contacting a population of neuronal cells in the subject with an effective amount of a nuclear factor of activated T cells (NFAT) antagonist.
2. The method of paragraph 1, wherein the population of neuronal cells is in proximity to an amyloid-beta deposit.
3. The method of any of paragraph s 1 to 2, wherein the contacting is performed by injection.
4. The method of any of paragraph s 1 to 3, wherein the effective amount is sufficient to decrease NFAT activity of one or more neuronal cells by at least about 5%, as compared to neuronal cells in the absence of the NFAT antagonist.
5. The method of paragraph 4, wherein the NFAT activity is mediated by calcineurin.
6. The method of any of paragraph s 1 to 5, wherein the effective amount is sufficient to increase dendritic spine density of one or more neuronal cells by at least about 5%, as compared to neuronal cells in the absence of the NFAT antagonist.
7. The method of any of paragraph s 1 to 6, wherein the effective amount is sufficient to decrease neuritic dystrophies of one or more neural cells by at least about 5%, as compared to neuronal cells in the absence of the NFAT antagonist.
8. The method of any of paragraph s 1 to 7, wherein the effective amount is about 10 mg/kg.
9. The method of any of paragraph s 1 to 8, wherein the NFAT antagonist is selected from the group consisting of a small molecule, a nucleic acid, a protein, a peptide, and an intrabody.
10. The method of any of paragraph s 1 to 9, wherein the NFAT antagonist is a peptide comprising an amino acid sequence of VIVIT (SEQ ID NO: 7).
The method of any of paragraph s 1 to 10, wherein the peptide further comprises a sequence encoding a nuclear localization signal.
12. The method of any of paragraph s 1 to 11, wherein the peptide is expressed by a vector.
13. The method of paragraph 12, wherein the vector is a viral vector.
14. The method of any of paragraphs 1 to 13, further comprising a step of diagnosing a subject with AD prior to the contacting.
15. The method of any of paragraph s 1 to 13, wherein the subject is a mammal.
16. The method of paragraph 15, wherein the mammal is a human.
17. A method of inhibiting neurodengeneration, comprising contacting a population of neuronal cells with an effective amount of a nuclear factor of activated T cells (NFAT) antagonist.
18. The method of paragraph 17, wherein the effective amount is sufficient to decrease NFAT activity of one or more neuronal cells by at least about 5%, as compared to neuronal cells in the absence of the NFAT antagonist.
19. The method of paragraph 18, wherein the NFAT activity is mediated by calcineurin.
20. The method of any of paragraphs 17 to 19, wherein the effective amount is sufficient to increase dendritic spine density of one or more neuronal cells by at least about 5%, as compared to neuronal cells in the absence of the NFAT antagonist.
21. The method of any of paragraphs 17 to 20, wherein the effective amount is sufficient to decrease neuritic dystrophies of one or more neural cells by at least about 5%, as compared to neuronal cells in the absence of the NFAT antagonist.
22. The method of any of paragraphs 17 to 21, wherein the effective amount is about 2 μM.
23. The method of any of paragraphs 17 to 22, wherein the NFAT antagonist is selected from the group consisting of a small molecule, a nucleic acid, a protein, a peptide and an intrabody.
24. The method of any of paragraphs 17 to 23, wherein the NFAT antagonist is a peptide comprising an amino acid sequence of VIVIT (SEQ ID NO: 7).
25. The method of paragraph 24, wherein the peptide further comprise a sequence encoding a nuclear localization signal.
26. The method of paragraph 24 or -25, wherein the peptide is expressed by a vector.
27. The method of paragraph 26, wherein the vector is a viral vector.
28. The method of any of paragraphs 17 to 27, wherein the contacting is in vitro.
29. The method of any of paragraphs 17 to 27, wherein the contacting is ex vivo.
30. The method of any of paragraphs 17 to 27, wherein the contacting is in vivo.
31. The method of paragraph 30, wherein the in vivo contacting is performed by injection.
32. The method of paragraph 30 or 31, wherein the in vivo contacting is in a subject diagnosed with or predisposed to a neurodegenerative disorder.
33. The method of paragraph 32, wherein the neurodegenerative disorder is Alzheimer's disease.
34. The method of paragraph 30 or 31, wherein the in vivo contacting is in a subject suffering from encephalitis or brain trauma.
35. The method of paragraph 30 or 31, wherein the in vivo contacting is in a subject suffering from tau-mediated synaptic degeneration.
36. The method of paragraph 30 or 31, wherein the in vivo contacting is in a subject suffering from frontotemporal dementia.
37. The method of any of paragraphs 32 to 36, wherein the subject is a mammal.
38. The method of paragraph 37, wherein the mammal is a human.
39. The method of any of paragraphs 17 to 38, wherein the population of neuronal cells is in proximity to an amyloid-beta deposit.

### EXAMPLES

The examples presented herein relate to inhibition of neurodegeneration with a NFAT antagonist, e.g., a VIVIT-containing peptide. In some embodiments, the NFAT antagonist can be administered to the brain for inhibiting neurodegeneration, e.g., in a subject diagnosed with AD or predisposed to AD. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the paragraphs to the invention, but are rather intended to be exemplary of certain embodiments. Any variations in the exemplified methods which occur to the skilled artisan are intended to fall within the scope of the present invention.

### Materials and Methods

Primary Neuronal Cultures.

The animals used for generating cell cultures were transgenic mice expressing human APP. Tg2576 line was generated from transgenic mice overexpressing the 695 aa isoform of human Alzheimer β-amyloid precursor protein containing the double Swedish mutation K670N, M671L with a hamster prion protein gene promoter in B6; SJL F2 mice (Hsiao et al., 1996). Primary neuronal cultures were derived from cerebral cortex of embryonic days 15-19 Tg2576 mice (Charles River Laboratories), as described previously in Wu et al. (2004) with modifications. Briefly, cortices were dissected, gently minced, trypsinized (0.027%, 37° C.; 5% CO₂ for 15 min), and then washed with 1×HBSS. Neurons were seeded to a density of 4×10⁵ viable cells/35 mm culture dishes previously coated with poly-D-lysine (100 μg/ml) for at least 1 h at 37° C. Cultures were maintained at 37° C. with 5% CO₂, supplemented with Neurobasal medium with 2% B27 nutrient, 2 mM L-glutamine, penicillin (100 U/ml), and streptomycin (100 μg/ml). The cultures were used within 28 d in vitro (DIV). To maintain elevated levels of extracellular Aβ, media were not changed. To identify the genotype of the animals, PCR was used on DNA extracted from sample tail taken after dissection of the cerebral cortex.

Adeno-Associated Viral Construction.

Adeno-associated viral (AAV) with an expression cassette of the chicken β-actin promoter driving enhanced green fluorescent protein (GFP), the mouse CaN isoform cDNA corresponding to wild type (CaNwt), the posttranslationally truncated form of CaNA encoding 45 kDa isoform (CaNCA), or AKAP79 peptide, flanked by the AAV inverted terminal repeats, was described previously in Spires et al. (2005). CaNwt, CaNCA (amino acid residues 1-399), and a peptide corresponding to human AKAP79 (hAKAP79) (amino acid residues 60-358) were subcloned into AAV-cytomegalovirus (CMV)/chicken β-actin (CBA)-woodchuck posttranscriptional regulatory element (WPRE) vector. Each construct was amplified by the following primers: (1) hemagglutinin (HA)-CaNwt-V5: 5′-GAATTCATGTATCCGTATGACGTACCAGAGTA-CGCCATGTCCGAGCCCAAGGCGATTGATCC (SEQ ID NO: 2); (2) HA-CaNwt-V5: 3′-GCTAGCTCACGTACTGTCGAGTCCCAGGAGAGGGTTTGGGATCGGCTTGCC-CTGGATATTGCTGCTATTACTGCCATTGC (SEQ ID NO: 3); (3) HA-CaNCA: CTAGTTCTGATGACTTCCTTCCGGGCTGCGGCCGTC (SEQ ID NO: 4); (4) Flag-hAKAP79: 5′-GAAGTTATCAGTCGACATGGACTACAAAGACGATGACGACA-AGGGCAGGAAGTGTCCACAA (SEQ ID NO: 5); and (5) Flag-hAKAP79: 3′-ATG-GTCTAGAAAGCTTCTAGACATTTTTAGATTTTGTAACATCAAATTCACTGATTTC (SEQ ID NO: 6). All the constructs were verified by sequencing.

Immunocytochemistry.

Immunocytochemistry was performed as described previously in Wu et al. (2004). Briefly, after being treated under different experimental conditions, cells were fixed with 4% paraformaldehyde in PBS, pH 7.4, for 15 min and were then membrane permeabilized with 0.5% Triton X-100 in PBS for 5 min. After blocking with 3% bovine serum albumin at 37° C. for 1 h, cells were incubated with primary antibodies: anti-microtubule-associated protein 2 (MAP2) antibody (1: 200; Sigma), or anti-NFATc4 antibody (1:200; Santa Cruz Biotechnology), anti-HA antibody (1:200; Invitrogen), or anti-Flag antibody (1:200; Sigma), at 4° C. overnight. Cells were then incubated with secondary antibodies conjugated to either cyanine 3 (Cy3) (1:500; Jackson ImmunoResearch) or Alexa 488 (1:500; Invitrogen). Fluorescent images were obtained using an LSM 510 Carl Zeiss microscope with a 25× or a 63× water-immersion objective lens. All images were taken at 512×512 pixel resolution. Nuclear NFATc4 staining was determined by overlap of NFATc4 staining with Hoechst nuclear staining. To calculate the NFATc4 ratio of nucleus versus cytoplasm, the intensity of nuclear NFATc4 was divided by the intensity of cytoplasmic NFATc4.

Immunohistochemistry.

Immunohistochemistry was performed as described previously in Spires et al. (2005). Briefly, animals were killed with an overdose of ketamine (30 mg/kg) and xylazine (3 mg/kg), and the brain was fixed in 4% paraformaldehyde in phosphate buffer with 15% glycerol cryoprotectant. Sections of 100 μm were cut on a freezing microtome, washed extensively in 0.1M phosphate buffer, and processed as free-floating slices. Permeabilization and blocking was achieved by incubation of the sections for 1 h at room temperature in 0.1M phosphate buffer, 0.5% Triton X-100 (v/v), and 3% bovine serum albumin. Primary antibodies for GFP (1:1000; Biogenesis), HA (1:100; Invitrogen), Flag (1:100; Sigma), and SMI312 (1:200; Sternberger Monoclonals) were applied overnight at 4° C. in 0.1 M phosphate buffer with 3% bovine serum albumin. After extensive washing, appropriate secondary antibodies conjugated to Cy3 (1:100 dilution), Alexa 488 (1:100 dilution), or Cy5 (1:100 dilution) were applied for 1 h at room temperature. Micrographs of immunostaining were obtained using a 20× objective with an upright Olympus Optical BX51 fluorescence microscope with an Olympus Optical DP70 camera or using a 63× water-immersion objective with a Carl Zeiss confocal microscope.

Preparation of Subcellular Fractionation.

Frozen tissue samples of human brain cortex (250 mg) were homogenized in 0.32M sucrose lysis buffer [0.32M sucrose, 5 mM CaCl₂, 3 mM Mg(acetate)₂, 0.1 mM EDTA, 10 mM Tris-HCl, pH 8.0, and 0.1% Triton X-100], supplemented with complete protease inhibitor cocktail tablets and centrifuged at 800-g for 15 min at 4° C. The supernatants were centrifuged at 100,000×g for 1 h at 4° C., and the resulting supernatants were regarded as the cytosolic fractions. The pellets from the initial centrifugation step were resuspended in 1.8M sucrose buffer containing 1.8 M sucrose, 3 mM Mg(acetate)₂, 1 mM DTT, and 10 mM Tris-HCl, pH 8.0, supplemented with complete protease inhibitor cocktail tablets (Roche Diagnostics). The nuclei were pelleted by centrifugation at 12,400×g for 1 h at 4° C. The pellet was resuspended in 0.32M sucrose buffer and washed by low-speed centrifugation. The final pellet was designated as the nuclear fraction.

For Western blot analyses, equal amounts of protein (20 μg) from each fraction were separated on a 4-20% SDS-PAGE gel. Primary antibodies for CaN (Stressgen) and NFATc4 (Santa Cruz Biotechnology) were used. Primary antibody incubation was followed by rabbit polyclonal horseradish peroxidase linked secondary antibody (1:1000; Bio-Rad). Immunoreactivity was visualized using enhanced chemiluminescence reagent (PerkinElmer Life and Analytical Sciences) and exposure on x-ray film. Anti-glyceraldehyde-3-phosphate dehydrogenase (GAPDH) antibody (Millipore Corporation) and anti-histone deacetylase 1 (HDAC1) antibody (Affinity BioReagents) were used to verify the integrity of the cytoplasmic and nuclear fraction separation.

Separation of Aβ Oligomers by Size Exclusion Chromatography.

Five milliliters of cultured media from 14 DIV wild-type or Tg neuron cultures was collected and centrifuged at 3000×g at 4° C. in Amicon Ultra-15ML 3K (Millipore Corporation) to concentrate proteins approximately fivefold. Concentrated cultured media (750 W) was separated by size exclusion chromatography (SEC) on Superdex 75 10/300 GL column (GE Healthcare) in 50 mM ammonium acetate, pH 8.5, with AKTA purifier 10 (GE Healthcare) (Townsend et al., 2006).

ToxiLight BioAssay.

Cell viability was determined in wild-type or Tg neurons at both 7 and 14 DIV using the ToxiLight BioAssay kit from Lonza. Preparation of cell extracts and the cytotoxicity assay were performed according to the protocol of the manufacturer.

Aβ Assay. Aβ levels were assayed from the medium collected from culture dishes at a different date, using an ELISA. Aβ concentration was determined with a sandwich ELISA kit (Wako) and with BAN50/BA27 for Aβ₄₀ and BAN50/BC05 for Aβ₄₂ in cultured medium and with BNT77/BA27 for Aβ₄₀ and BNT77/BC05 for Aβ₄₂ in fractionated samples by size-exclusion chromatography. Samples was optimized to detect Aβ in the range of 6.25-100 fmol/ml. ELISA signals were reported as the mean±SD of two replica wells in femtomoles of Aβ per milligram of protein (determined with the BCA Protein Assay Reagent kit; Pierce).

Immunoprecipitation Aβ from Cultured Medium.

Cultured medium of wild-type or Tg cultures at 14 DIV were collected. Medium was centrifuged at 1200 rpm for 5 min at 4° C. to remove cellular debris. Supernatants (1.3 ml) for each condition were precleared with 50 μl of protein G Sepharose (Sigma) for 1 h at 4° C. with gentle shaking and then centrifuged at 15,000 rpm at 4° C. for 5 min. Supernatants were incubated with 5 μg of anti-6E10 antibody or 5 μg of anti-GFP (Abeam) as control. After an overnight incubation at 4° C., protein G Sepharose (60 μl) was added and incubated at 4° C. for 2 h. Beads were isolated by centrifugation (15,000 rpm, 4° C. for 5 min) and subsequently washed three times with TBS buffer before the addition of 2× Laemli's sample buffer.

[Ca²⁺]ᵢ Measurements.

Primary neurons were plated on 35 mm glass-bottomed dishes (MatTek) and maintained in a standard Neurobasal medium without Phenol Red. Calcium levels were evaluated in wild-type neurons treated for 24 h with wild-type or Tg conditioned media as well as with transgenic conditioned media that was pre-depleted with 3D6 antibody. In all cases, calcium imaging was performed using Indo-1 as described previously in Bacskai et al. (2003). Briefly, Indo-1/AM (Invitrogen) was dissolved with 20% pluronic F-127 (Invitrogen) in DMSO and then added to the culture dishes at a final concentration of 1 μM Indo-1/AM and 0.02% pluronic F-127 for 45 min. Cells were imaged with an Olympus Optical Fluoview 1000 MPE with pre-chirp optics and a fast acousto-optical modulator mounted on an Olympus BX61WI upright microscope and an Olympus Optical 20× dipping objective (numerical aperture, 0.95). A mode-locked titanium/sapphire laser (MaiTai; Spectra Physics) generated two-photon fluorescence with 750 nm excitation, and the emitted light was discriminated into two channels with interference filters corresponding to 390 nm, 65 nm bandpass and 495 nm, 20 nm bandpass (Chroma Technology) for ratiometric imaging. The capture settings remained unchanged for the entire experiment. Fluorescence emission ratios were calculated in the neuronal cell bodies. To convert the calculated ratios into calcium concentrations, primary neurons were incubated with Indo-1/AM and treated with either calcium-free or 39 μM calcium buffers in the presence of 20 μM ionomycin for 15 min. The calcium-free and calcium-saturated ratios were then measured and used as the Rₘᵢₙ and Rₘₐₓ. These ratios along with the K_{D} of Indo-1 for calcium of 250 nM (Grynkiewicz et al., 1985) were used for calculation of calcium concentration.

Luciferase reporter assay. CaNwt, CaNCA, or NFAT-TA-Luc (Clontech) was subcloned into AAV serotype 2 vector. These plasmids (1 μg) were used as primary neurons at 3 DIV. After 24 h, the cells were harvested and luciferase activities were measured with a luminometer using a reagent kit (Luciferase Assay System with Reporter Lysis Buffer; Promega). The background luciferase activity was subtracted from all experiments.

Experimental Animals.

C57BL/6J wild-type mice and double transgenic mice (B6C3APPswe/PS1dE9 line; The Jackson Laboratory) overexpressing mutant human APP and mutant human Presenilin 1 (PS1), as well as transgenic mice expressing human Swedish mutated APP (Tg2576 line) were used. These mice were housed in the animal facility, and C57BL/6J wild-type and APP/PS1 mice were used at the age of 5-6 months for intracortical injection. All experiments were performed in accordance with animal protocols approved by the Institutional Animal Care and Use Committee.

Intracortical Injections and Surgery.

Intracortical injections and surgery were performed as described previously (Spires et al., 2005; Kuchibhotla et al., 2008; Meyer-Luehmann et al., 2008). Briefly, mice were anesthetized with ketamine (10 mg/kg) and xylazine (1 mg/kg) and placed in a stereotaxic apparatus. The surgical site was sterilized with betadyne and isopropyl alcohol, and a 2-3 mm incision was made in the scalp along the midline between the ears. Burr holes were drilled in the skull, 0.5 mm posterior from bregma, and 0.5 mm lateral to the midsagittal line. Using a Hamilton syringe, 3 μl of virus (titer, 4.2×10¹² viral genomes/ml) was injected 1.2 mm deep in somatosensory cortex at a rate of 0.25 μl/min. After one injection in each burr hole, the scalp was sutured, and the mouse recovered from anesthesia on a heating pad.

Three to 4 weeks after injection, APP/PS1 transgenic mice received an intraperitoneal injection of methoxy-XO₄ (10 mg/kg), a fluorescent compound that crosses the blood-brain barrier and binds to amyloid plaques (Klunk et al., 2002). A cranial window of 6 mm in diameter was installed under anesthesia (10 mg/kg ketamine and 1 mg/kg xylazine). At the same time, Texas Red dextran (70,000 Da molecular weight; 12.5 mg/ml in sterile PBS; Invitrogen) was injected into a lateral tail vein to provide a fluorescent angiogram.

Multiphoton Imaging.

Images of GFP-filled neuronal processes and, in the case of APP/PS1 mice, amyloid pathology, and blood vessels were obtained using a Bio-Rad 1024ES multiphoton microscope (Bio-Rad), mounted on an Olympus Optical BX50WI upright microscope. A wax ring was placed on the edges of the cover-slip of the cortical window and filled with distilled water to create a well for an Olympus Optical 20× dipping objective (numerical aperture, 0.95). A mode-locked titanium/sapphire laser (MaiTai; Spectra Physics) generated two-photon fluorescence with 800 nm excitation, and detectors containing three photomultiplier tubes (Hamamatsu) collected emitted light in the range of 380-480, 500-540, and 560-650 nm (Bacskai et al., 2003). GFP-filled neuronal processes were sampled ˜100 μm below the surface of the brain around somatosensory cortex. At the end of imaging sessions, mice were allowed to recover and placed singly in their home cage.

Labeling of Fibrillar Amyloid Deposits.

After immunohistochemistry, fibrillar amyloid deposits (neuritic plaques) were stained for 8 min in a solution of thioflavin S (2 μg/ml) in 0.1 M PBS and then rinsed with ddH2O.

Statistical Analyses.

Data are presented as mean±SD. Comparisons were made between groups by one- or two-way ANOVA, followed by post hoc Bonferroni's test for comparison among means. Differences with a p value of <0.05 were considered statistically significant.

### Example 1

### Abnormal Morphologies in Neurons from Tg2576 Cultures

Neuritic abnormalities have been seen surrounding plaques in human AD and in aged APP-overexpressing mouse models of Alzheimer's Disease (AD), but studying these lesions is difficult because they are distributed apparently randomly throughout the cortical mantle, occur only in aged animals, and the location of new lesions cannot be predicted. Presented herein is a tractable model for neuronal abnormalities associated with AD. It was first sought to examine whether mature primary neurons from transgenic embryos expressing APP with the familial Swedish mutation (Tg2576 line) develop any of the morphological phenotypes associated with neurodegeneration in the intact (aged) transgenic brain and in human AD, including dendritic spine loss, diminished dendritic complexity, and neuritic dystrophies. To assess neuronal morphology, cultured neurons were transfected with GFP to label individual neurons. During the course of 21 days in vitro (DIV), GFP-positive neurons from Tg2576 (Tg) cultures progressively develop a neurodegenerative phenotype when compared with neurons from wild-type mice. In wild-type neurons at 14 DIV, GFP fluorescence demonstrated intricately branched dendritic arbors studded with protrusions that included mature spines and few fillopodia (FIGS. 1A and 1B). However, in Tg neurons, the maturation is accompanied by an increase in the number of neurons with focal neuritic swellings (FIGS. 1C and 1D). The Tg neurons exhibit simplified dendritic complexity and localized dendritic dystrophies. The difference is marked; for example, at 14 DIV (FIG. 1E), the number of neurons with beaded neurites in Tg cultures is about sevenfold higher than those in wild-type cultures. In addition, the number of neurons with dystrophic neuritis increased with time (FIG. 1E). In Tg cultures, GFP-positive neurons with dystrophies are found in ˜14% of neurons at 14 DIV and in ˜24% at 21 DIV.

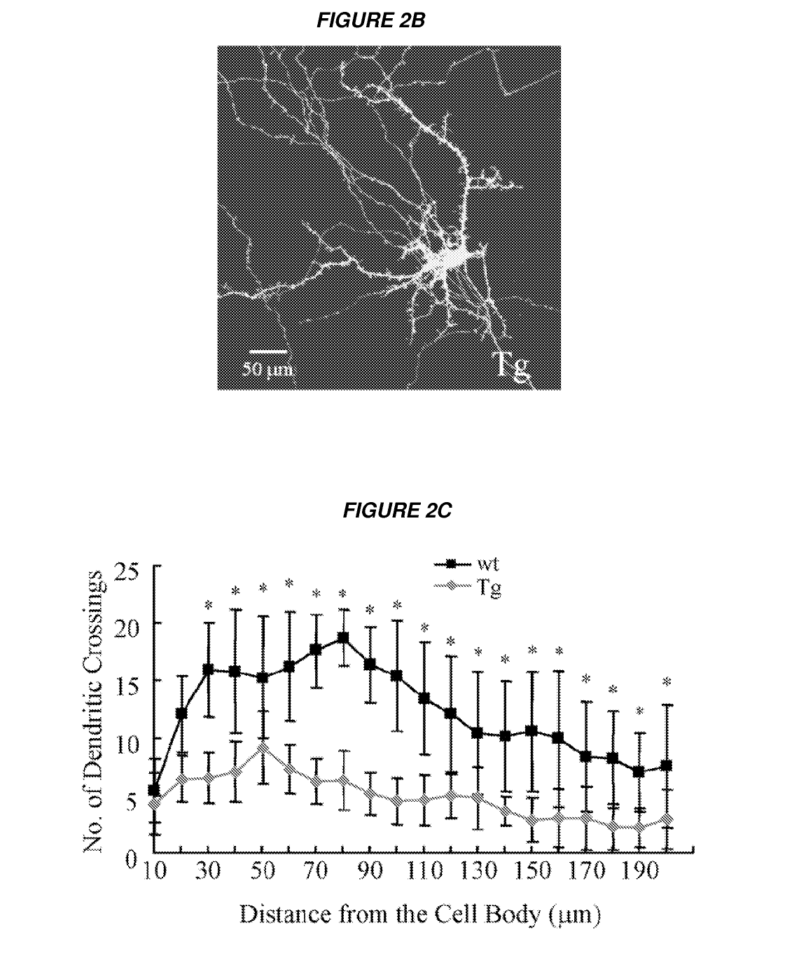

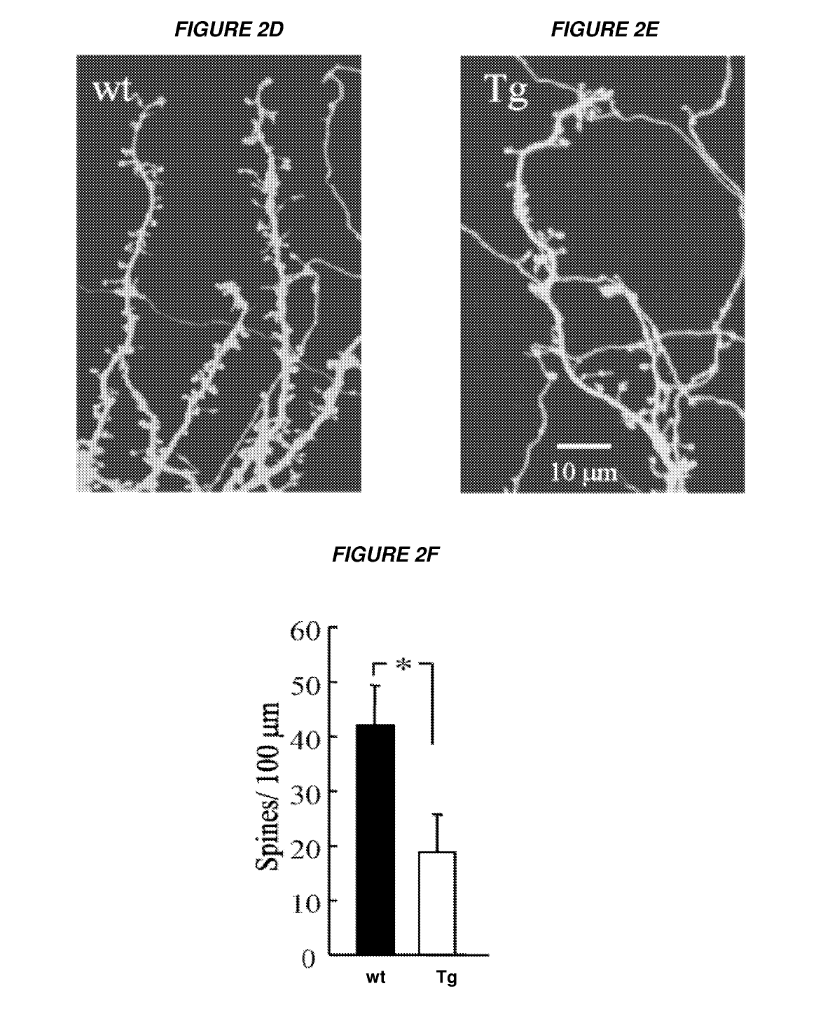

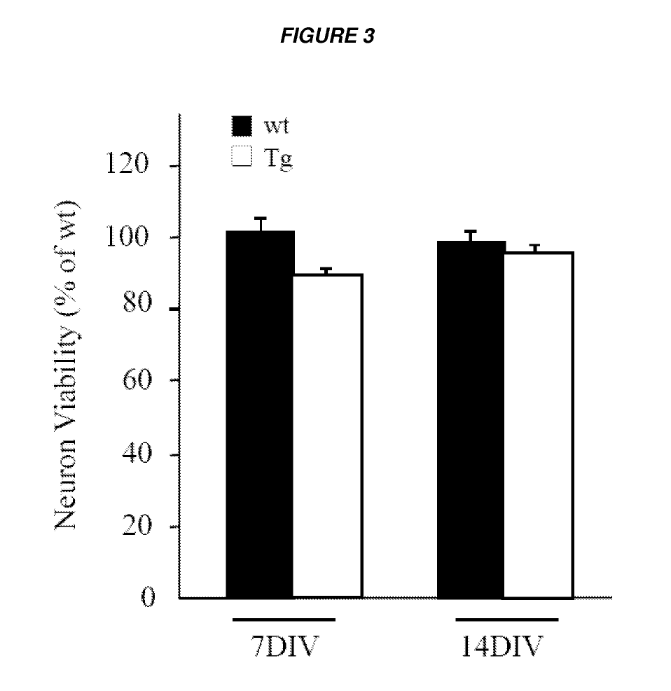

Because some morphological changes other than dendritic dystrophies, such as decreased dendritic branches and spine density, are also observed in the early stages of neurodegeneration, dendritic branching and the density of dendritic spines of GFP-positive neurons from Tg and wild-type cultures were compared. As measured by Sholl analysis, neurons in Tg cultures had reduced dendritic complexity at all points farther than 30 μm from the cell body compared with wild-type cultures (FIGS. 2A to 2C). These effects are not attributable solely to processes with morphological changes like dystrophies. For example, comparison of spine density in neurons (excluding neurons with dystrophies) between Tg and wild-type cultures showed that the mean spine density of Tg neurons is markedly lower than that of wild-type neurons (FIGS. 2D to 2F). These effects do not appear to be attributable to toxicity; no typical apoptotic nuclei (chromatin condensation) appeared in Tg neurons compared with wild-type neurons at 14 DIV, when both the Tg and wild-type neurons were labeled with anti-microtubule-associated protein 2 (MAP2) and Hoechst nuclear counterstain (data not shown). ToxiLight assay for cell death showed no significant difference in cell viability between wild-type and Tg neurons at either 7 or 14 DIV (FIG. 3). Together, these findings indicate that neurons with abnormal morphologies are limited in wild-type cultures, whereas in Tg cultures, neurons develop dystrophies, profound dendritic simplification, and loss of dendritic spines

### Example 2

### Conditioned Media (CM) from Tg Culture Contains Oligomeric Aβ and Induces Elevation of [Ca²⁺]ᵢ

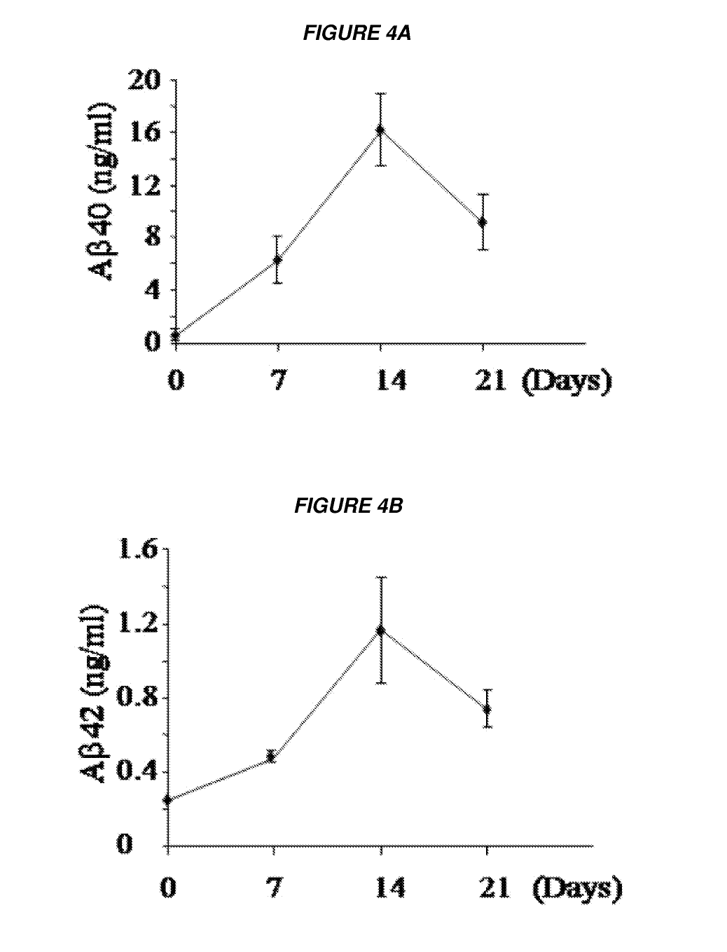

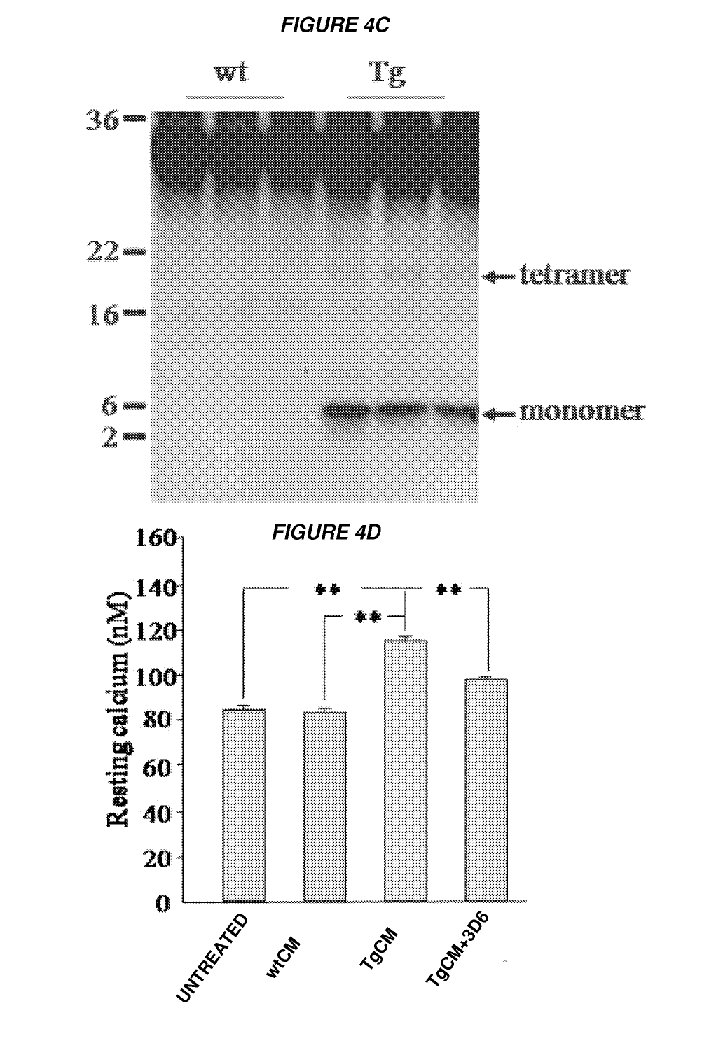

Primary cortical neurons derived from Tg embryos at 14 DIV produced high levels of two major types of human Aβ peptides, Aβ₄₀ and Aβ₄₂; the concentration of Aβ₄₀ was 16 ng/ml and of Aβ₄₂ was 1.2 ng/ml as determined by ELISA (FIGS. 4A and 4B). Moreover, Western blot analysis of immunoprecipitation revealed the presence of readily detectable SDS-stable small oligomers in CM of Tg cultures at 14 DIV, similar to those reported to be synaptotoxic (Shankar et al., 2008) (FIG. 4C).

Neurites in the region near senile plaques have been recently observed to contain high [Ca²⁺]ᵢ (Kuchibhotla et al., 2008). To examine whether Aβ is capable of inducing an increase in [Ca²⁺]ᵢ, the effect of Tg neuronal culture CM on calcium homeostasis of wild-type neurons was assessed. Wild-type cortical neurons were cultured in standard NB/B27 serum-free medium, and, at 14 DIV, the medium was replaced with diluted 1:2 CM from wild-type (wtCM) or Tg (TgCM) cultures and further incubated for 24 h. As shown in FIG. 4D, wild-type neurons treated with TgCM for 24 h exhibited elevated levels of [Ca²⁺]ᵢ compared with neurons maintained in wild-type CM for 24 h. There was no significant difference in [Ca²⁺]ᵢ levels between untreated neurons and neurons treated with wtCM for 24 h, indicating that the elevated levels of [Ca²⁺]ᵢ is specifically resulted from TgCM. To determine whether Aβ caused the elevation of the [Ca²⁺]ᵢ, [Ca²⁺]ᵢ was compared between neurons applying TgCM with or without immunodepletion using 3D6 antibody, a high-titer monoclonal Aβ antibody (Johnson-Wood et al., 1997). 3D6 immunodepletion completely prevented the elevation of [Ca²⁺]ᵢ (FIG. 4D).

### Example 3

### CaN-NFATc4-Aberrant Nuclear Localization in Neurons from Tg Cultures and Human AD Postmortem Brain

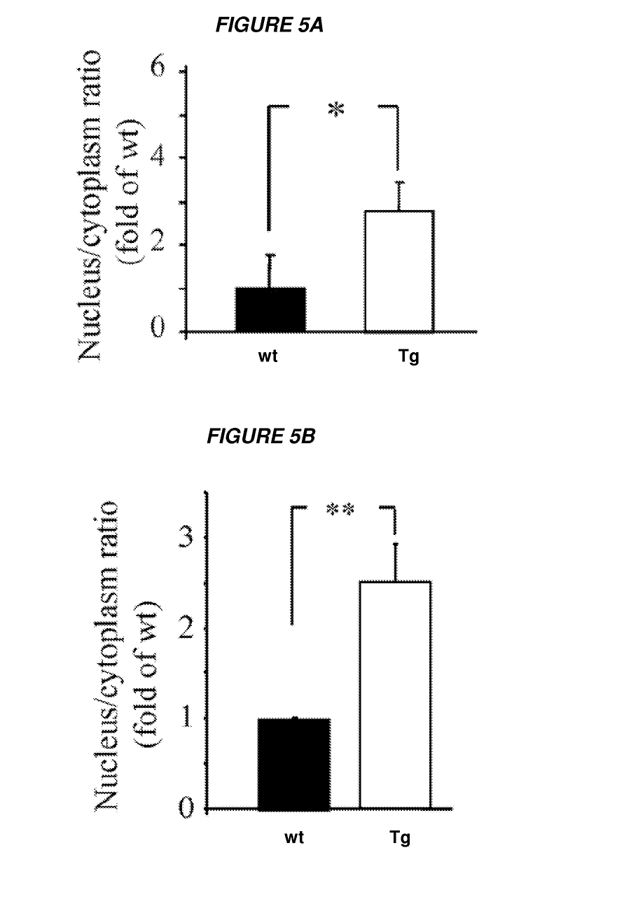

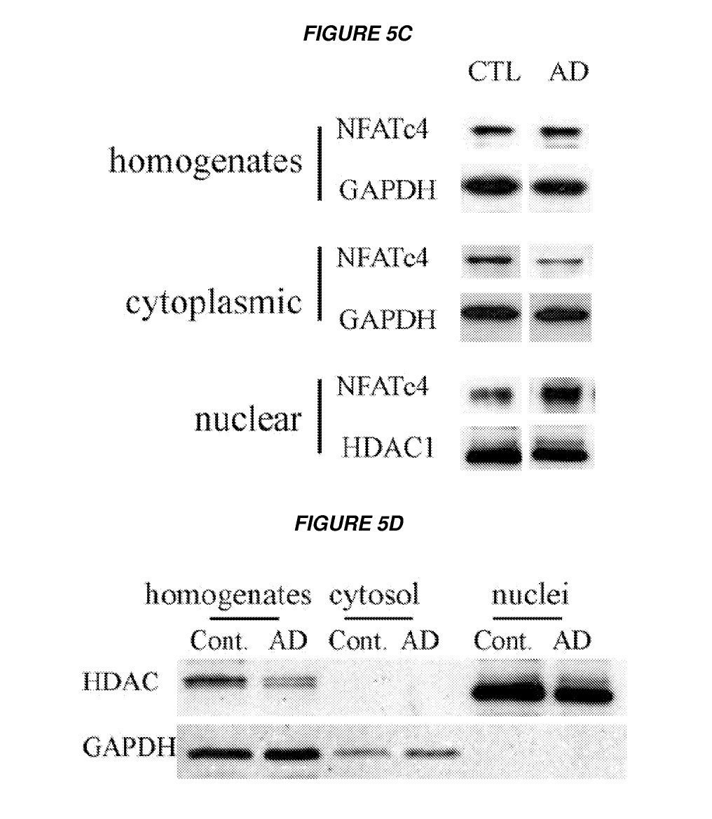

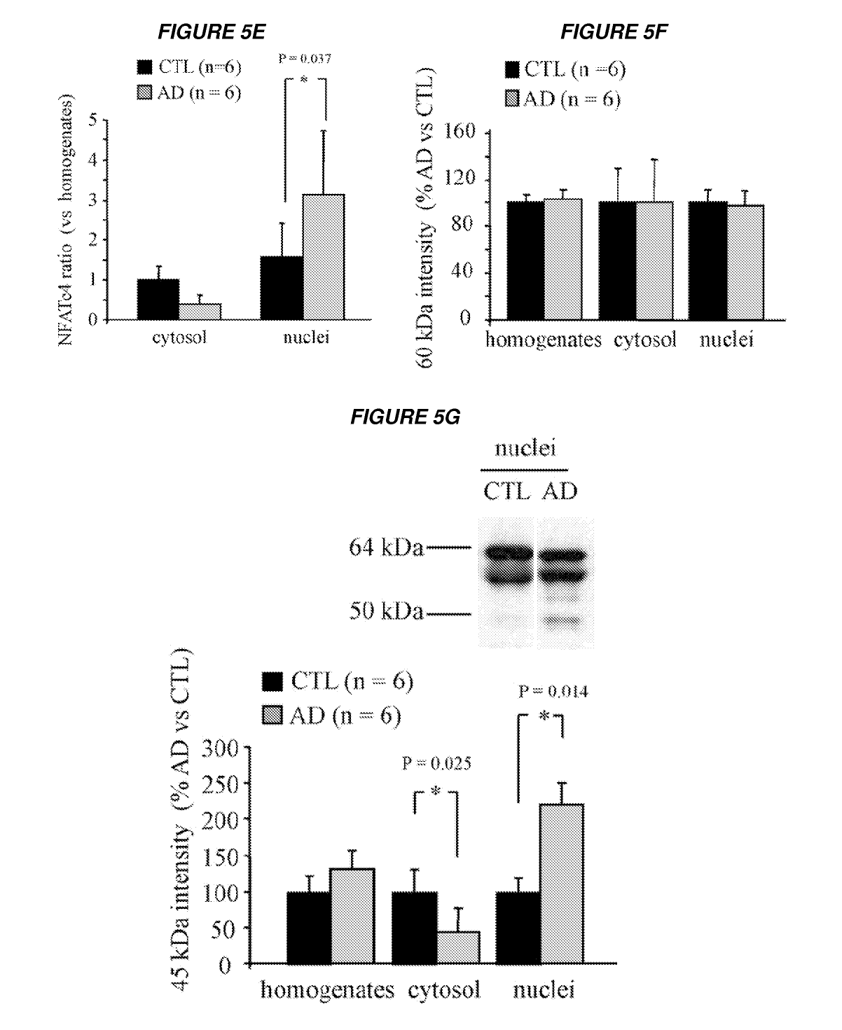

Because Aβ in TgCM induces elevated [Ca²⁺]ᵢ in cultured neurons and Hyman B T et al. have previously shown that neurites with abnormal morphologies in both APP/PS1 and APP transgenic mice were strongly associated with [Ca²⁺]ᵢ overload (Kuchibhotla et al., 2008), it was sought to examine Ca²⁺-mediated pathways as the link between exposure to Aβ and a neurodegenerative phenotype. Because CaN is the most calcium-sensitive protein phosphatase in the brain (Klee et al., 1979), CaN activity was examined in neurons from Tg cultures to determine if it is upregulated. NFATc4, the nuclear factor of activated T cells, is a well known CaN substrate. Activation of the predominant neuronal NFATc4 isoform, which is abundantly expressed in cortical neurons, can be determined by its nuclear translocation after CaN-mediated dephosphorylation. Cultured neurons at 14 DIV were stained with an antibody against endogenous NFATc4, a Hoechst counterstain for identification of nuclei, and MAP2, a neuron marker. Compared with wild-type neurons, Tg neurons show higher immunoreactivity of NFATc in the nuclei. The NFATc4 immunofluorescence intensity was measured, and the ratio of the intensity (nucleus vs cytoplasm) was compared between neurons from wild-type and Tg cultures. Very little colocalization of Hoechst nuclear staining and NFATc4 was observed in wild-type neuron cultures, indicating that inactive NFATc4 is mostly located in the cytoplasm, whereas in Tg culture, NFATc4 staining was enhanced in nuclei. (Quantification result is shown in FIG. 5A; NFATc4 and MAP2 images used for quantification of nucleus/cytoplasm ratio of NFATc4 are not shown.) Similarly, CaN is known to translocate to the nucleus when activated, and Tg neuron cultures showed higher immunofluorescence intensity of CaN in the nucleus compared with wild-type neurons. (Quantification result is shown in FIG. 5B; Hoechst and CaN images used for quantification of nucleus/cytoplasm ratio of CaN are not shown.) These data show that CaN and NFAT are activated in Tg neurons.

To determine whether the aberrant nuclear localization of CaN-NFATc4 observed in Tg neurons could be relevant to the AD human condition, total homogenates, cytosol, and nuclei were prepared from frozen human AD or control cortex tissue samples and analyzed by immunoblotting with antibodies against endogenous NFATc4 or CaN. The human sample information is shown in Table 1.

TABLE 1
Human Sample Information

| Group | N | Male/Female | Age (years) | Dementia |
|---|---|---|---|---|
| Control | 1 | F | 77 | No |
| Control | 2 | F | 78 | No |
| Control | 3 | M | 104 | No |
| Control | 4 | F | 87 | No |
| Control | 5 | F | 63 | No |
| Control | 6 | F | 97 | No |
| AD | 1 | F | 72 | Yes |
| AD | 2 | F | 58 | Yes |
| AD | 3 | F | 63 | Yes |
| AD | 4 | F | 84 | Yes |
| AD | 5 | F | 78 | Yes |
| AD | 6 | M | 57 | Yes |

NFATc4 immunoreactivity in the nuclear fraction prepared from AD samples was markedly increased compared with that observed in control samples, but no changes were observed in either the total homogenate or cytoplasmic fractions (FIGS. 5C to 5E). Immunoblot analysis with CaN showed similar intensity of full-length CaN in all three compartments between AD and control (FIG. 5F). In contrast, a consistent 2.2-fold increase in the level of a 45 kDa fragment of CaN, a posttranslationally truncated form of CaN (CaNCA), which is constitutively active, was observed in the nuclear fraction in AD samples compared with control samples (FIG. 5G). This proteolytic product removes the autoinhibitory C-terminal domain and has been observed as a mechanism of CaN activation in stroke, trauma, and glaucoma (Morioka et al., 1999; Wu et al., 2004; Burkard et al., 2005; Huang et al., 2005). Together, the aberrant accumulation of nuclear NFATc4 and constitutively active form of CaN indicate aberrant activation of CaN-NFATc4 signaling in AD. These results confirm the observation of truncated CaN in AD by Liu et al. (2005) but appear to contrast with those of Celsi et al. (2007) who observed diminished CaN immunoreactivity in neurons in AD, perhaps because the latter study did not distinguish full-length from truncated forms.

### Example 4

### Conditioned Media of Tg Culture and Aβ Oligomers Isolated by SEC Induce NFATc4 Nuclear Translocation in Wild-Type Neurons

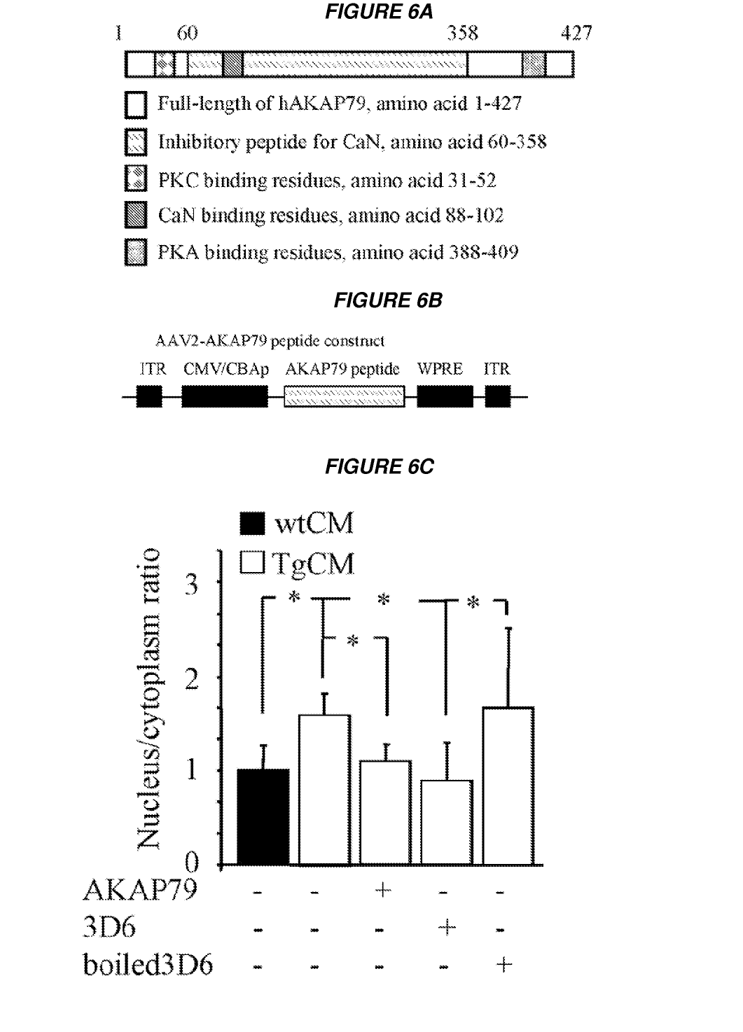

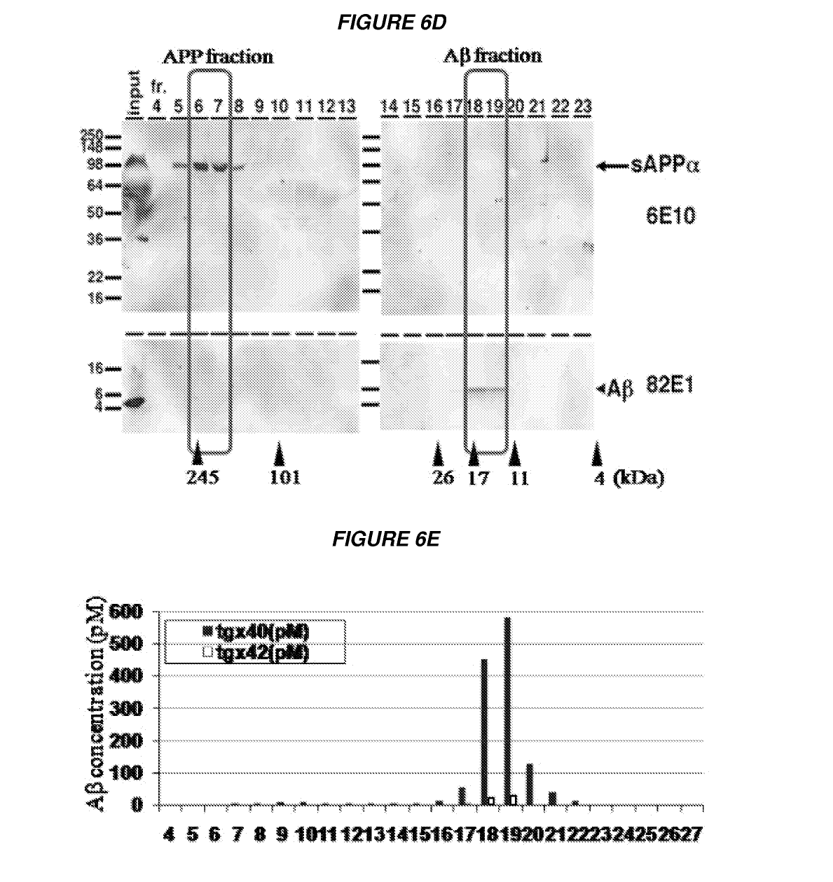

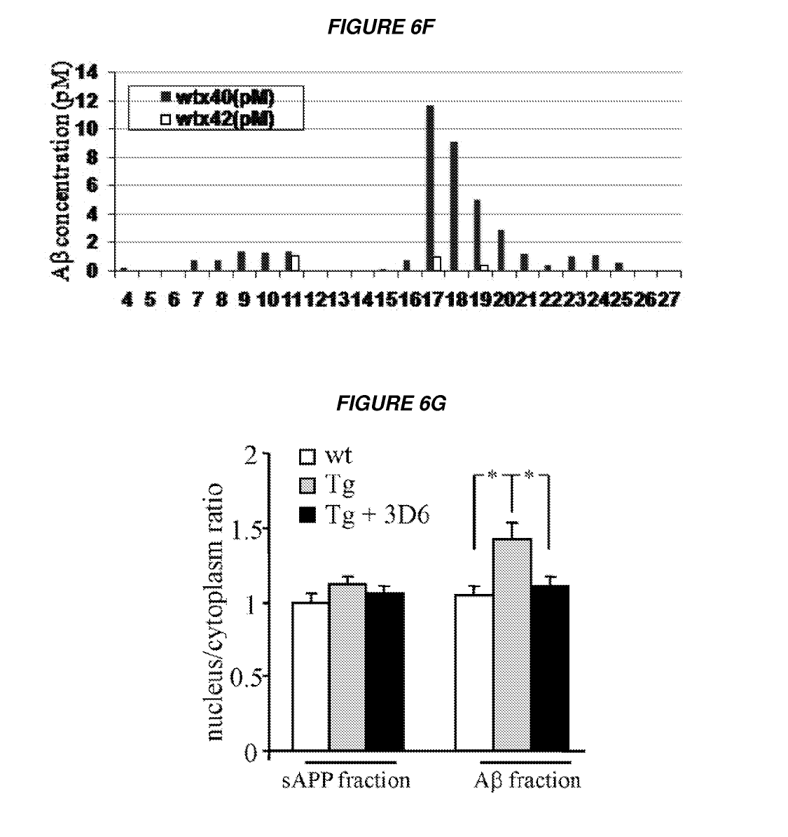

Soluble plaque-associated bioactive molecules, possibly assemblies of Aβ, may be responsible for the “halo” effect of neuritic change and [Ca²⁺]ᵢ alterations near plaques (Kuchibhotla et al., 2008; Meyer-Luehmann et al., 2008; Koffie et al., 2009). To examine this possibility, the effect of Tg neuronal culture conditioned medium on CaN activation and neurite properties of wild-type neurons was investigated. Wild-type cortical neurons were cultured in standard NB/B27 serum-free medium, and, at 14 DIV, the medium was replaced with diluted 1:4 CM from wild-type or Tg cultures for 24 h. The cultures were then stained with NFATc4, MAP2 and Hoechst nuclear counterstain. As shown in FIG. 6C, neurons treated with wtCM for 24 h exhibited a relatively low nucleus/cytoplasm ratio of NFATc4, which was similar to neurons in culture without CM replacement. In contrast, neurons treated with TgCM for 24 h demonstrated a significant increase in the nucleus/cytoplasm ratio of NFATc4 compared with neurons maintained in wtCM. The increased ratio was considerably reduced in neurons overexpressing an adeno-associated virus encoding an epitope-tagged (Flag) AKAP79 fragment (AAV-AKAP79), a potent CaN inhibitory peptide (Coghlan et al., 1995) (FIGS. 6A and 6B). Immunodepletion of Aβ from the TgCM with 3D6 prevented the increase in the nucleus/cytoplasm ratio of NFATc4 (FIG. 6C), suggesting that soluble Aβ species is involved in the TgCM-mediated activation of CaN and NFATc4 nuclear translocation. (NFATc4 and MAP2 images used for quantification of nucleus/cytoplasm ratio of NFATc4 are not shown.) To better characterize the specific role of Aβ oligomers in CaN-mediated NFATc4 activation, nondenaturing size exclusion chromatography (SEC) was used to isolate the various Aβ species from TgCM. Both ELISA and Western blot analysis showed that SEC fractions 17-20 of TgCM, but not the same fractions of wtCM, were markedly enriched in either oligomeric forms of Aβ40 and Aβ42 (FIGS. 6D to 6F). Application of SEC fractions 18-19 (oligomeric Aβ fraction) of TgCM onto wild-type neurons for 24 h caused a significant increase in translocation of NFATc4 to the nucleus, but no changes were observed in neurons treated with the same SEC fractions of wtCM. Immunodepletion of Aβ from the fractions 18-19 of TgCM with 3D6 prevented the increase in the nucleus/cytoplasm ratio of NFATc4 (FIG. 6G). Wild-type neurons treated with fractions 6-7 [soluble APP (sAPP) fraction] (FIGS. 6D to 6F) from either TgCM or wtCM showed no significant difference in the nucleus/cytoplasm ratio of NFATc4. These results suggest that either Aβ-containing CM from Tg culture or SEC-isolated Aβ oligomers is capable of inducing CaN-mediated NFATc4 activation.

### Example 5

### A β-Containing Conditioned Medium Causes Morphological Abnormalities in Wild-Type Neurons Identical to Those Observed in Tg Neurons in Culture

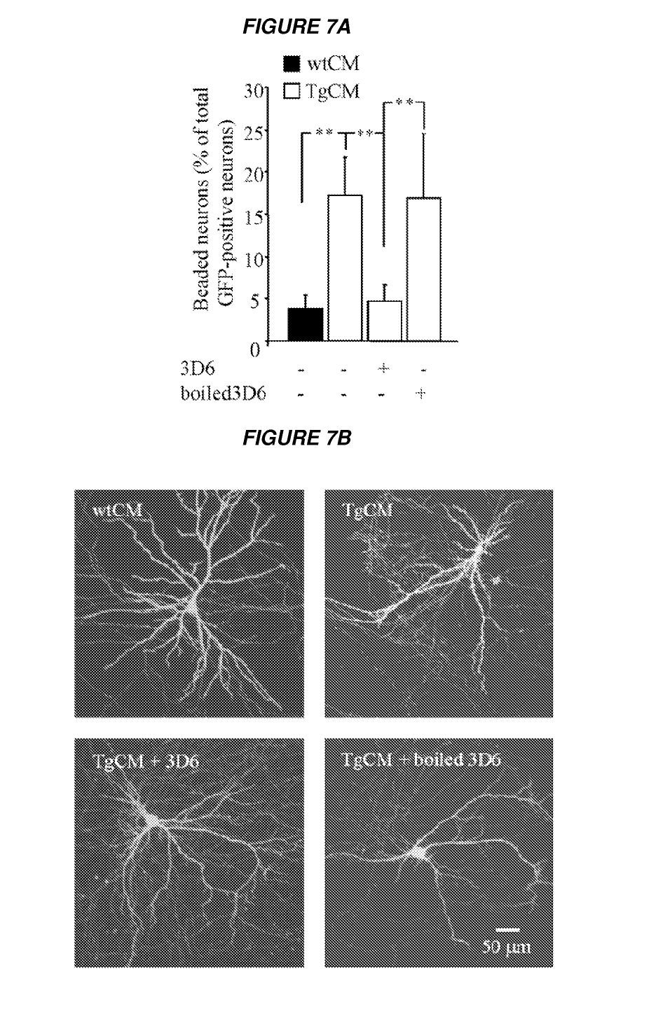

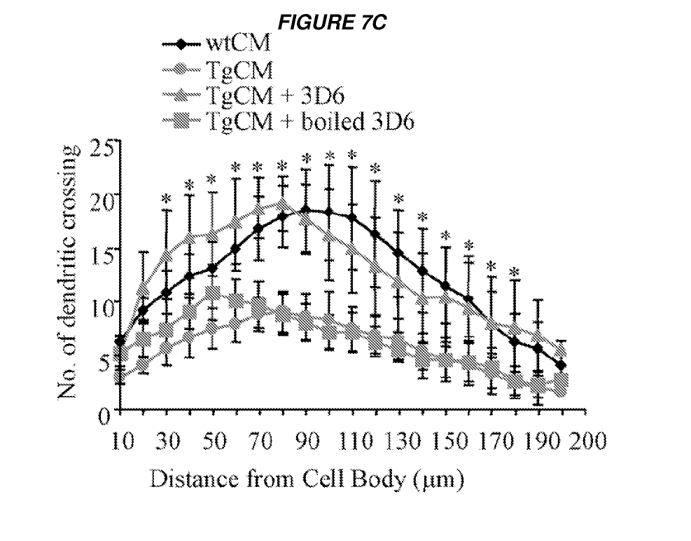

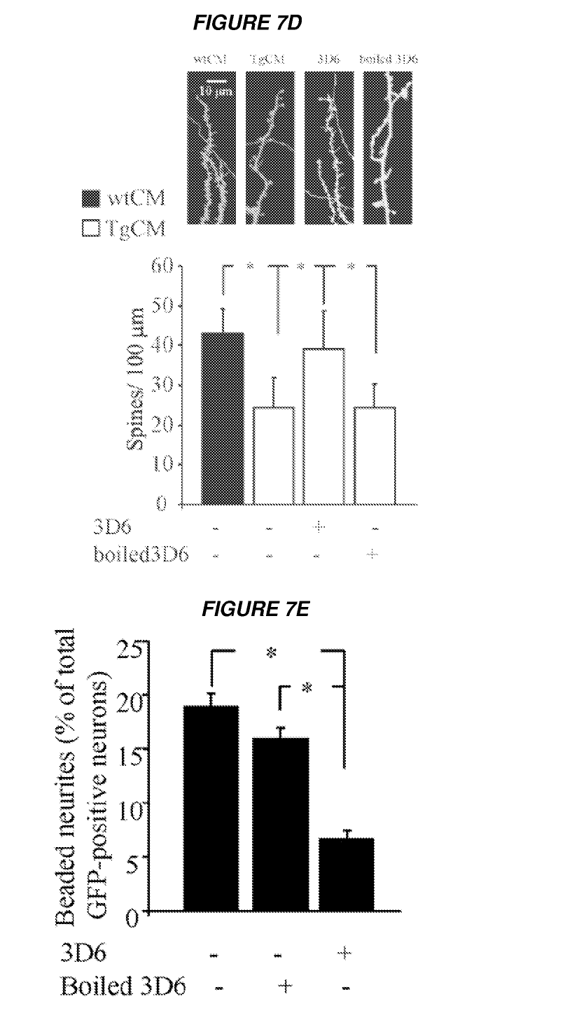

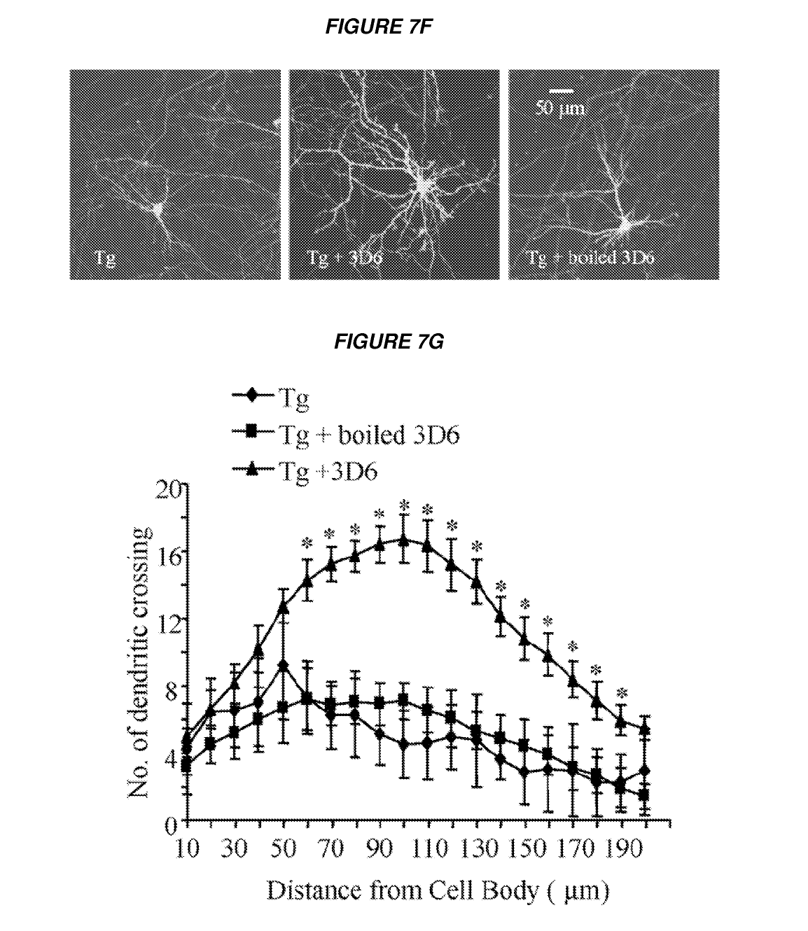

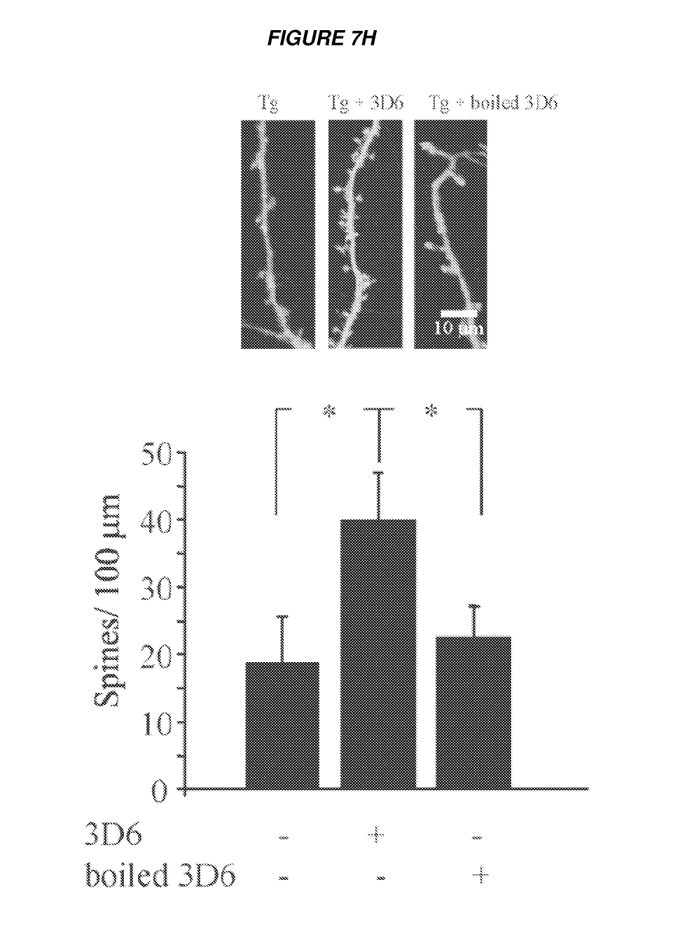

To assess whether the abnormal morphology observed in Tg cultures is caused by Aβ, it was sought to examine wild-type cultured cortical neurons growing in either Tg or wildtype CM starting 24 h after plating and maintained in CM until neuron maturity (24 DIV). As shown in FIG. 7A, wild-type cultures maintained in TgCM exhibited a significantly higher number of neurons with focal neuritic dystrophies compared with cultures maintained in wtCM. Cultures maintained in TgCM that was preimmunodepleted by 3D6, but not boiled 3D6, had a lower number of neurons with dystrophies similar to that seen in cultures growing in wtCM. Sholl analysis indicated that neurons growing in Aβ-containing TgCM also had dendritic simplification, but this is not the case in cultures maintained in TgCM that was immunodepleted by 3D6 (FIGS. 7B and 7C). A striking effect of TgCM on dendritic spine density was also noted. The density of dendritic spines in the neurons growing in TgCM was greatly decreased (FIG. 7D). There was no significant reduction of spine density between neurons growing in wtCM and TgCM that was immunodepleted by 3D6, whereas spine density from neurons growing in TgCM that was treated with inactive, boiled 3D6 was also significantly reduced. In addition, Tg neurons grown in media immunodepleted with 3D6 at 3 DIV no longer developed dystrophic morphology during maturation. No statistically significant difference was detected in the number of neurons with dystrophies (FIG. 7E), dendritic complexity (FIGS. 7F and 7G), or the mean spine density (FIG. 7H) between wild-type and Tg neurons (that overexpress APP) treated with media that had been immunodepleted by 3D6. These results suggest that Aβ immunodepletion blocks neurodegenerative morphologies that occur in neurons exposed to Aβ-containing CM or that overexpress APP.

### Example 6

### Inhibition of Either CaN Activity or CaN-Mediated NFAT Activation Abolishes TgCM- or APP Overexpression-Induced Morphological Abnormalities

Both CaN-mediated NFATc4 activation and abnormal morphologies were detected in Tg neurons and in wild-type neurons growing in TgCM. It was reasoned that Aβ might cause the morphological changes via CaN activation, or CaN activation and morphologic changes might each be unrelated effects of Aβ treatment. To address whether CaN is involved in the development of Aβ-related abnormal morphologies, wild-type neurons growing in TgCM at 3 DIV were transduced with an AAV encoding a CaN inhibitory peptide AKAP79. To characterize the wild-type neurons infected by AAV-AKAP79, the cells were stained for MAP2 and flag-tagged AKAP79. At 14 DIV, essentially 100% of neurons expressed AKAP79 by measuring the colocalization of Flag-tag-positive neurons and MAP2-stained neurons (data not shown). Tg neurons overexpressing AKAP79, but not a control vector (i.e. AAV-β-galactosidase), contained significantly fewer neurons with beaded processes at 21 DIV (FIG. 8A). AKAP79 overexpression abolished TgCM-induced dendritic simplification as assessed by Sholl analysis (FIGS. 8B and 8C) and reduction in dendritic spine density (FIG. 8D). In addition, Tg neurons overexpressing AKAP79 inhibitory peptide at an early time point (3 DIV) no longer developed dystrophic morphology during maturation. No statistically significant difference in the number of neurons with dystrophies (FIG. 8E), dendritic complexity (FIGS. 8F and 8G), and the mean spine density (FIG. 8H) were detected between wild-type and Tg neurons overexpressing AKAP79 inhibitory peptide. FK506, a potent inhibitor of CaN with an independent mechanism of action, also blocked TgCM-induced dendritic spine loss (FIG. 8I). These results indicate that CaN inhibition blocks neurodegenerative morphologies that occur in neurons exposed to Aβ-containing CM or that overexpress APP.

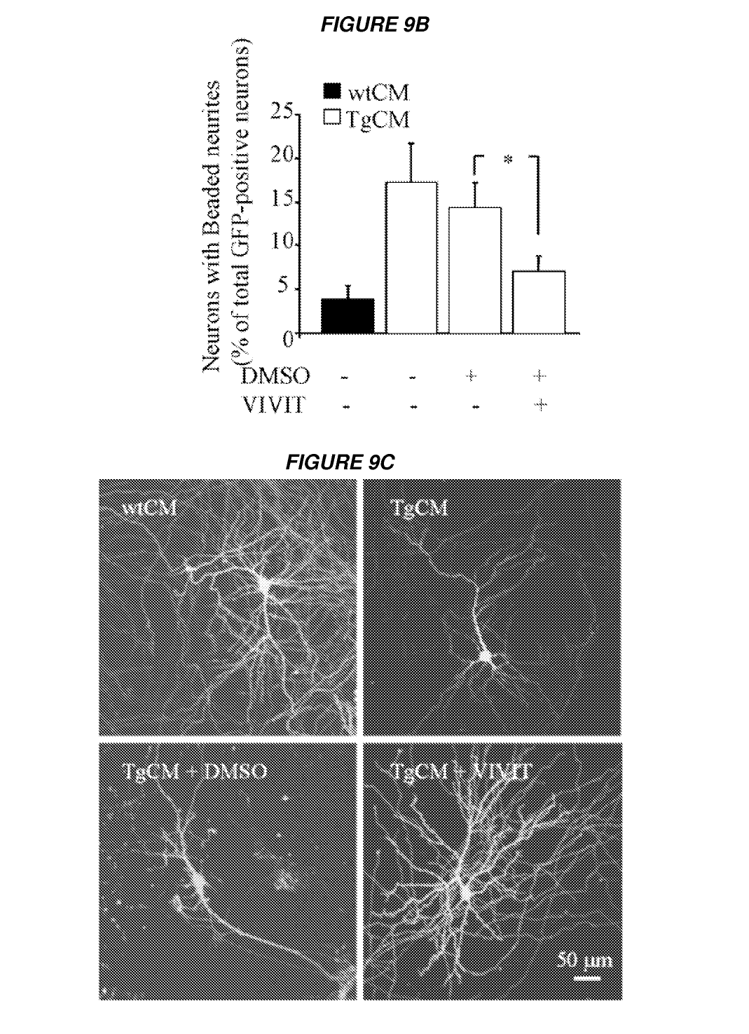

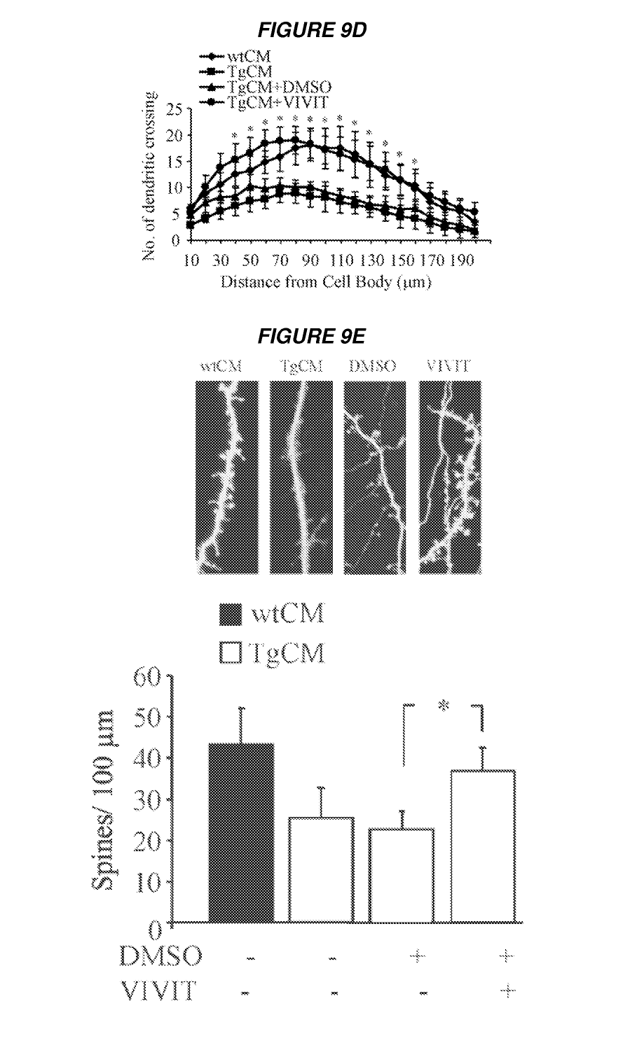

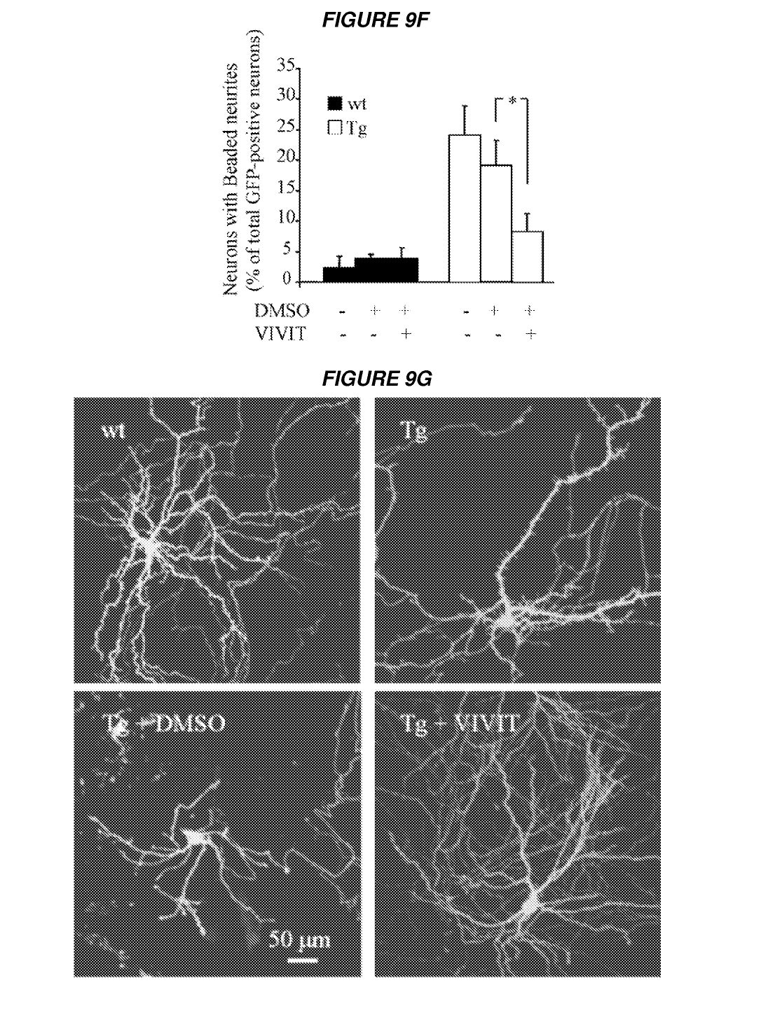

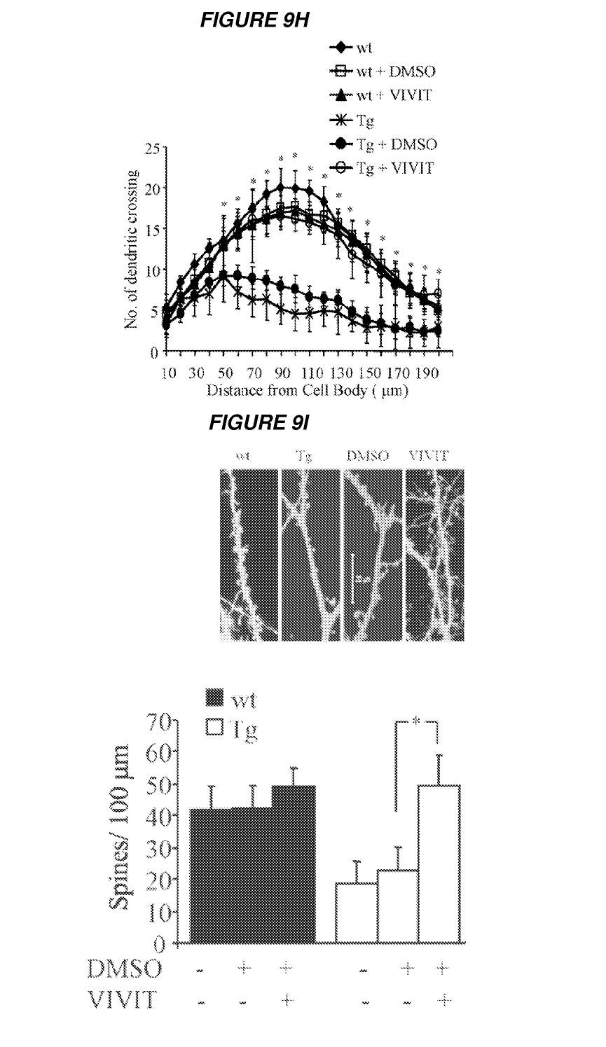

CaN-mediated activation of NFAT has been demonstrated to play an important role in axonal outgrowth and activity dependent dendritic structural changes during development (Graef et al., 2003; Groth and Mermelstein, 2003; Schwartz et al., 2009). The findings presented herein indicate that nuclear accumulation of NFATc4 occurs in neurons from Tg cultures and in human AD postmortem brain and that Aβ-containing CM from Tg culture is capable of inducing CaN-mediated NFATc4 nuclear localization in wild-type neurons. To assess the signaling pathway activated by CaN that can involve CaN-dependent morphological neurodegeneration during Aβ neurotoxicity, the effect of 11R-VIVIT, a cell-permeable VIVIT peptide that blocks the docking sites for CaN-NFAT interaction and therefore specifically inhibits CaN-mediated NFAT activation without impacting other CaN substrates (Aramburu et al., 1999), was examined. Application of 2 μM VIVIT (SEQ ID NO: 7) to wild-type neurons growing in TgCM abolished TgCM-induced NFAT nuclear accumulation (Quantification result is shown in FIG. 9A; NFATc4 and DAPI images used for quantification of nucleus/cytoplasm ratio of NFATc4 are not shown.) and blocked morphological deficits induced by Aβ, including reduced numbers of neurons with dendritic dystrophies (FIG. 9B), increased dendritic complexity (FIGS. 9C and 9D), and spine density (FIG. 9E). Similarly, Tg neurons treated with VIVIT (SEQ ID NO: 7) showed dramatically improved morphology compared with Tg neurons treated with carrier (DMSO) (FIGS. 9F to 9I). No significant changes in the number of neurons with dendritic dystrophies, dendritic processes, and spine density were detected in wild-type neurons treated with VIVIT (SEQ ID NO: 7) or DMSO, suggesting that inhibition of CaN-NFAT interaction, e.g., at a concentration of 2 μM, does not have a strong effect on baseline maturation. Thus, inhibition of CaN-NFAT interaction and subsequent NFAT nuclear accumulation by VIVIT (SEQ ID NO: 7) blocks TgCM or APP overexpression-induced morphological abnormalities in culture. These data indicate that upregulation of CaN activity by Aβ activates the NFAT cascade, resulting in a pathological triad of dendritic spine loss, dendritic simplification, and neuritic dystrophies.

### Example 7

### Transduction of a Constitutively-Active CaN (CaNCA) into Wild-Type Cultured Neurons Induces Abnormal Morphology that is a Phenocopy of the Aβ Effect

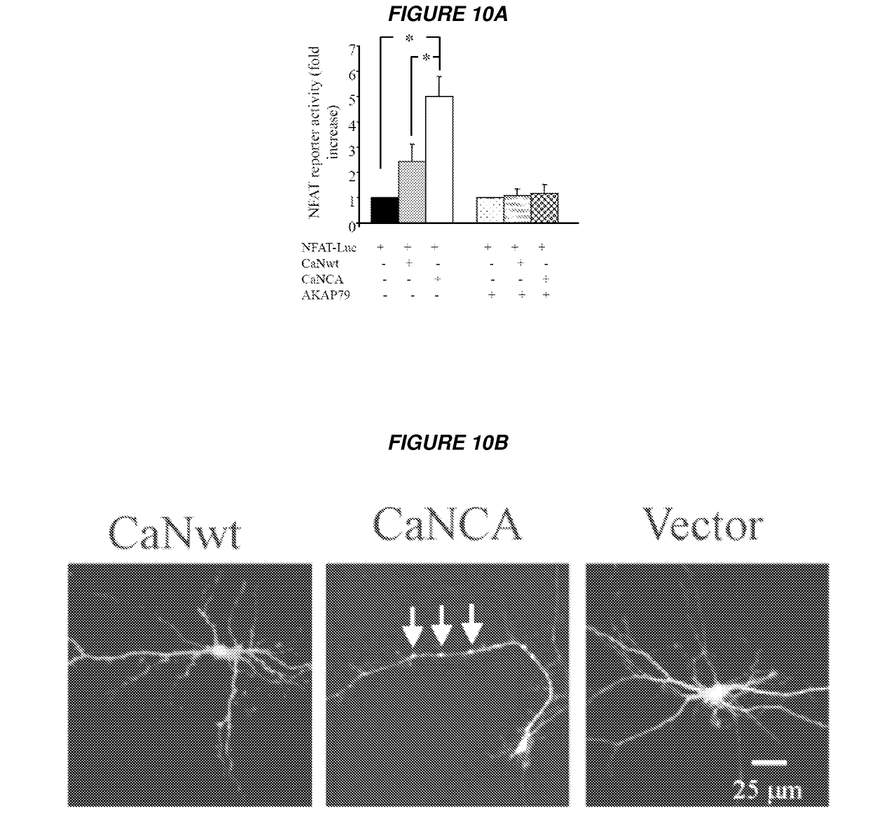

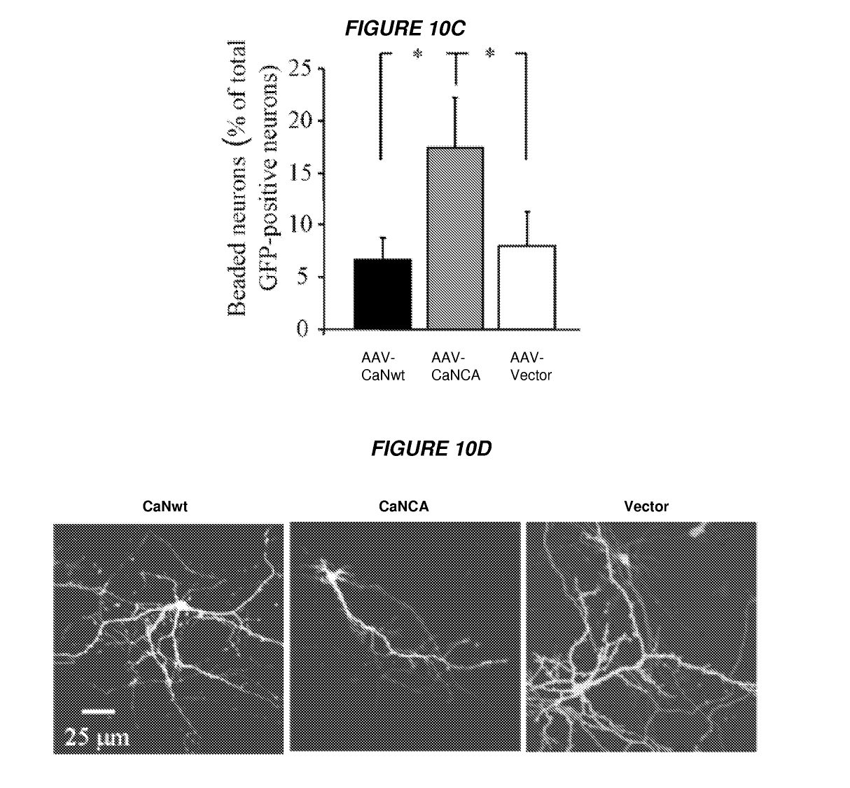

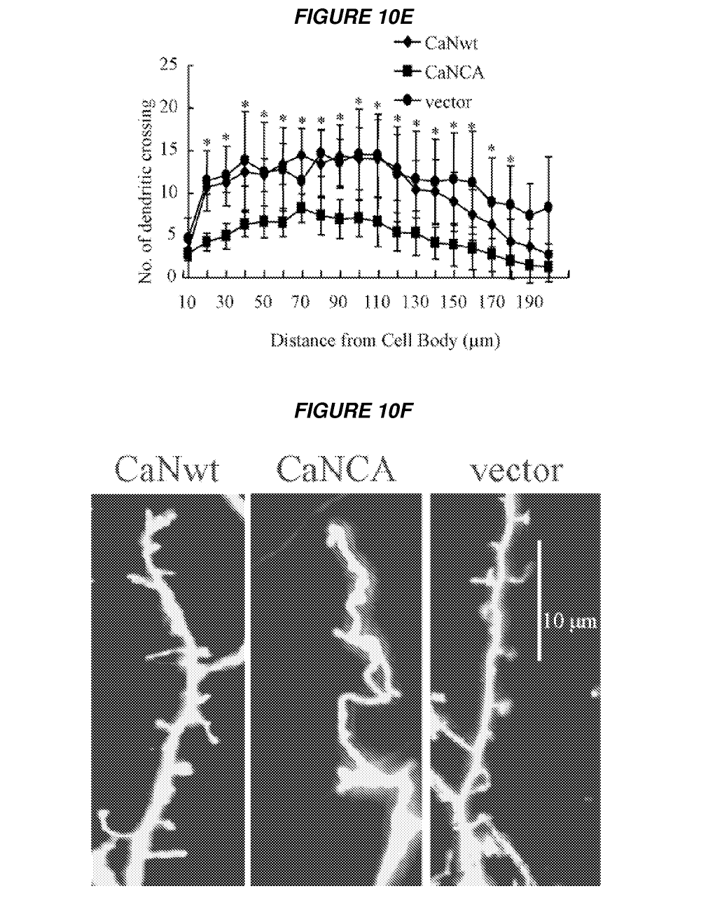

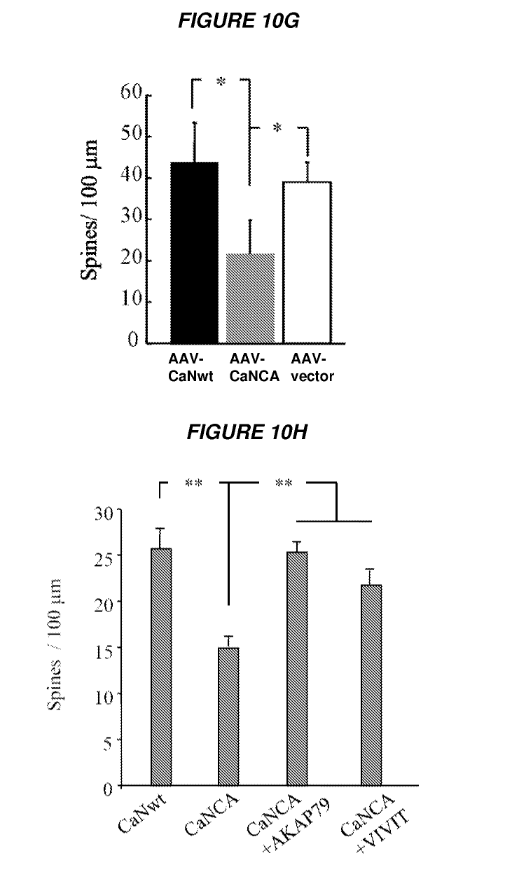

To examine whether CaN activation is sufficient to cause these morphologic changes, even in the absence of overexpressed APP or exogenously applied Aβ, it was sought to investigate whether the expression of a constitutively-active CaN (CaNCA) can favor morphological changes in wild-type cultured neurons. An AAV vector encoding an HA-tagged CaNCA (AAV-CaNCA) or wild-type CaN (AAV-CaNwt) or an AAV vector control was expressed in cortical cultures. HA-tag immunostaining showed that nearly all MAP2-stained neurons expressed AAV-CaNwt or AAV-CaNCA (data not shown), and neurons overexpressing CaNCA had significantly increased NFAT-luciferase activity, which was blocked by AKAP79 inhibitor peptide (FIG. 10A). Neurons with swollen dendrites were barely detectable in CaNwt-overexpressing cultures, but they were prominently detected in CaNCA-overexpressing cultures (FIG. 10B). Approximately 15% of total GFP-positive neurons were observed to have typical local neuritic dystrophies in cultures overexpressing CaNCA, whereas 6% of total GFP-positive neurons developed dystrophies in CaNwt-expressing cultures (FIG. 10C). The latter is similar to the number of dystrophic neurites found in cultures expressing β-galactosidase, an AAV-vector control. Dendritic complexity (FIGS. 10D and 10E) and mean spine density (FIGS. 10F and 100) were reduced in neurons overexpressing CaNCA but not in CaNwt- or vector-overexpressing neurons. Treatment of the neurons with AKAP79 inhibitory peptide or VIVIT (SEQ ID NO: 7) can virtually eliminate CaNCA overexpression-induced dendritic spine loss (FIG. 10H), suggesting that NFAT activation is critical for CaNCA-induced dendritic spine loss. These data indicate that ectopic expression of CaNCA is sufficient to cause neuritic dystrophies, dendritic simplification, and spine loss in neurons in culture.

In addition, the inventors have demonstrated that neurons in culture that over-express tau or a fragment of tau, previously observed in both Alzheimer's disease and frontotemporal dementia, leads to calcineurin activation (as assessed by translocation of endogenous NFAT to the nucleus), and a similar phenotype of dendritic alterations as demonstrated herein by other ways of activating calcineurin (i.e., via constitutively active calcineurin or via exposure to oligomeric Alpha-beta) (Data not shown). Thus, the findings indicate that tau triggers calcineurin/NFAT activation and neurodegeneration.

### Example 8

### CaNCA Induces Abnormal Neuronal Morphologies In Vivo

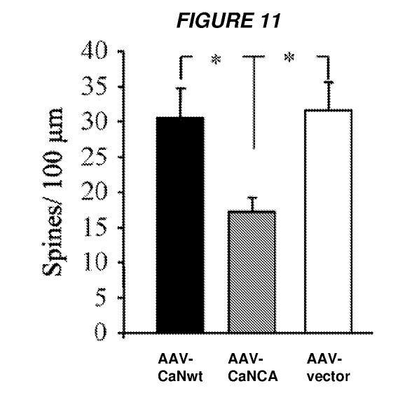

To investigate the role of elevated CaN activity in neuronal morphology in the intact adult mouse brain, high-titer AAV-CaNCA, AAV-CaNwt, or AAV-β-galactosidase, along with AAV-GFP, was introduced intracranially into the somatosensory cortex or hippocampus of C57BL/6J mice by stereotaxic injection, as described in Materials and Methods. Three to four weeks after intracranial injection, GFP-filled neurites and corresponding spines were detectable in the live animals using multiphoton microscopy (data not shown) (Spires et al., 2005). GFP-labeled neurons were also observed in limited cortical and hippocampal areas in postmortem sections stained with a GFP antibody (data not shown). Confirmed with HA-tag staining, almost all of the GFP-positive cells were immunoreactive for CaNwt (or CaNCA), showing that GFP-labeled neurons expressed CaNwt or CaNCA (data not shown). Compared with neurons expressing control vector or CaNwt, CaNCA-expressing neurons showed high levels of NFATc4 nuclear distribution (data not shown). The dendritic morphology of GFP-labeled neurites from cortical and hippocampal areas of wild-type mice injected with CaNCA displayed neurodegenerative alterations. Dystrophies were observed along the length of dendrites (data not shown) or axons (data not shown), whereas dendrites or axons from these areas of mice injected with CaNwt showed normal processes (data not shown), similar to those seen in vector control-injected mice. High-power images from multiphoton observations of the live brain allowed analysis of dendritic spines, which showed that, compared with the control, spine density was significantly decreased in mice injected with CaNCA, whereas CaNwt-injected mice showed mean spine density comparable with control vector-injected mice (Quantification result is shown in FIG. 11; Images for quantification of spine densities are not shown).

### Example 9

### Overexpression of a Genetically Encoded CaN Inhibitor AKAP79 Inhibitory Peptide Reduces Aβ-Associated Neurodegenerative Alterations In Vivo

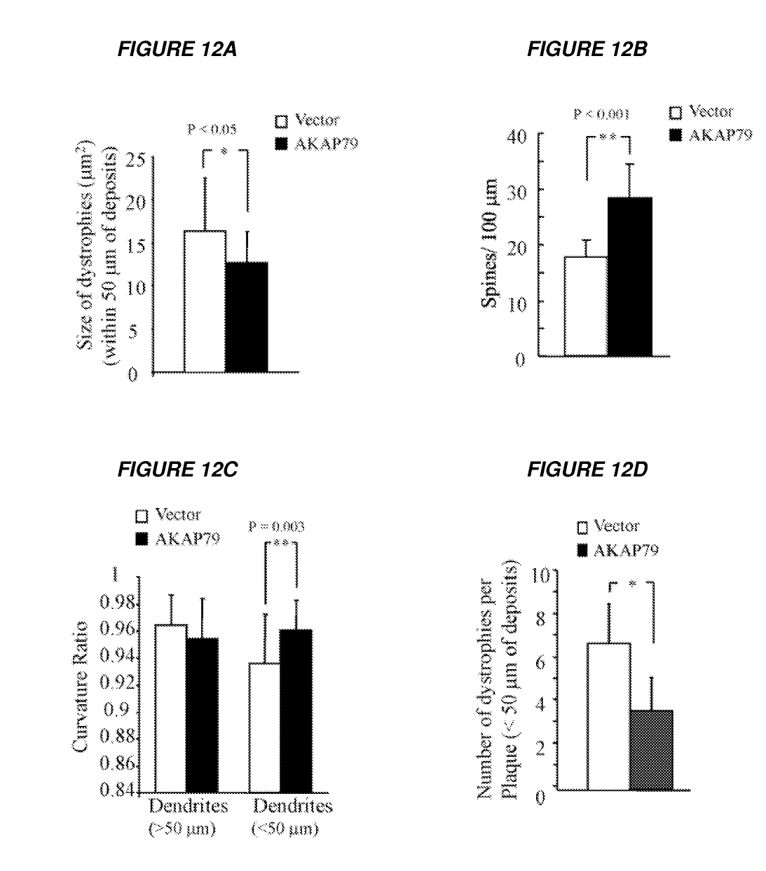

To directly determine the effect of CaN inhibition on the Aβ-related morphological abnormalities in the adult rodent brain in vivo, AAV-AKAP79 inhibitory peptide or a vector control was coinjected with AAV-GFP into somatosensory cortex in 6-month-old living APP/PS1 mice. With multiphoton imaging, neuritic dystrophies (size of dystrophies defined as areas of swelling >2.5 μm in diameter), neurite curvature, as well as spine density near to (<50 μm) or far from (>50 μm) amyloid deposits were quantitatively compared in living APP/PS1 mice. In vivo low-magnification image shows GFP-expressing neuritis and axons, blood vessels containing Texas Red, and amyloid deposition stained with methoxy-XO₄ (data not shown). High-magnification live images taken from ˜100 μm below the brain surface in the neocortex of adult APP/PS1 mice show dendritic spines and dystrophies in both vector- and AKAP79-expressing conditions (data not shown). These images were used for quantification of dendritic dystrophies, spine density, and neurite curvature in both vector- and AKAP79-expressing mice. The mean size of dystrophies near plaques from control vector-injected brain was significantly larger than that observed in brain injected with AKAP79 (FIG. 12A). Compared with the vector control, spine density was significantly increased near plaques in mice injected with AKAP79 inhibitory peptide (FIG. 12B).

Another well characterized morphological alteration in both APP overexpressing mouse brain and human AD is the development of tortuous, nonlinear trajectories for neurites, especially near plaques (Knowles et al., 1999; Le et al., 2001). This tortuosity is measured by “neuronal curvature,” a marker of how straight a neurite segment is (Knowles et al., 1999). Compared with the elevated neuritic curvature seen at baseline in the APP/PS1 mice, a significant improvement in neurite curvature was observed in AKAP79 inhibitory peptide-injected mice (FIG. 12C). Both GFP and AKAP79 inhibitory peptide (flag-tagged) were coexpressed in the vast majority of neurons (data not shown). Examination of Aβ deposits revealed no changes in the Aβ deposits themselves associated with AAV injection.

To verify in vivo imaging of GFP in neuritic dystrophies, SMI312 immunoreactivity, which recognizes a neurofilament protein that labels all axons, were examined in postmortem sections. The postmortem sections indicative of axonal dystrophies with SMI312 staining and plaques with thioflavin S show that plaque-associated axonal dystrophies are reduced in areas injected with AKAP79 (data not shown). In the postmortem samples, axons in control areas injected with the vector (i.e., not expressing AKAP79 inhibitory peptide) in close proximity to Aβ deposits had abnormal large dystrophies (data not shown). In contrast, substantially fewer axons in the AKAP79 inhibitory peptide injected areas in close proximity to Aβ deposits showed dystrophies (Quantification result is shown in FIG. 12D; Images for quantification of plaque-associated axonal dystrophies are not shown). Consistent with the live imaging data, the mean size of dystrophies from control vector-injected brain was significantly larger than those observed in areas injected with AKAP79 inhibitory peptide (data not shown). Together, these results show that CaN inhibition blocks the neurodegenerative changes that occur in the immediate vicinity of Aβ plaques in vivo, without affecting the Aβ deposits.

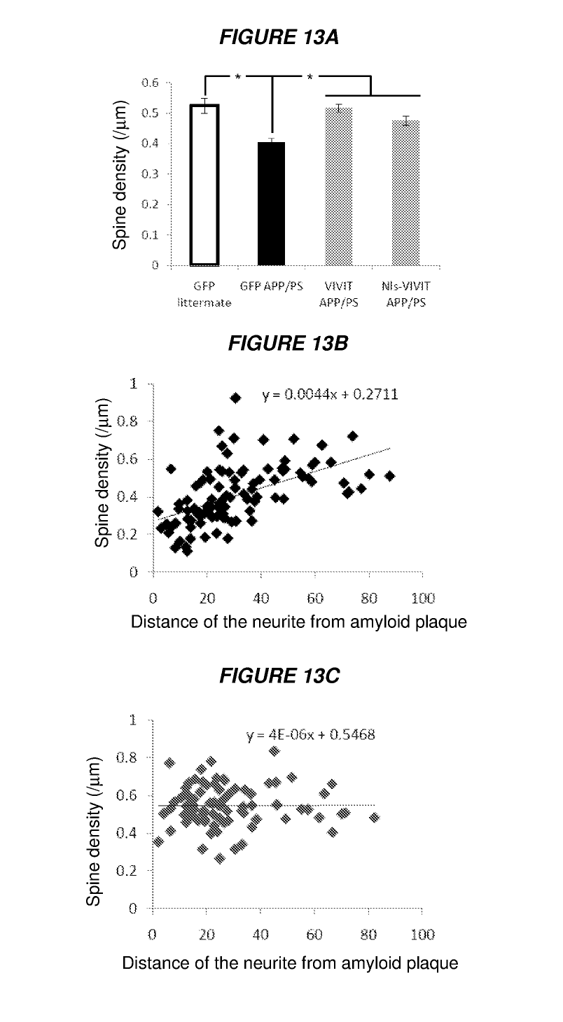

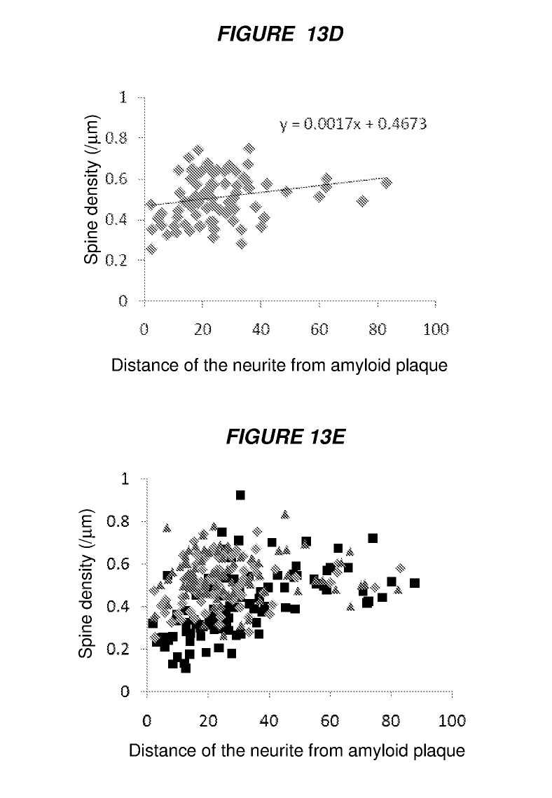

The inventors have also demonstrated that overexpression of a genetically encoded NFAT inhibitor VIVIT-containing inhibitory peptide improves dendritic spine density in neurons surrounding amyloid plaques in an in vivo AD model mouse (FIGS. 13A to 13E).

### Discussion

Although it has been clear for a century that amyloid plaques are surrounded by neuritic abnormalities and the amyloid hypothesis of AD suggests that Aβ peptide induces downstream neurodegenerative changes leading ultimately to collapse of neural networks and clinical dementia (Hardy and Allsop, 1991; Hardy and Selkoe, 2002), the molecules and mechanisms underlying the translation of Aβ neurotoxicity into morphological disruption remain undefined. Presented herein demonstrates that activation of CaN and subsequent NFAT-mediated downstream cascades are key molecular mechanisms linking Aβ to damage of the structural underpinnings of neural networks. This molecular model of neuronal degeneration is initiated by Aβ-inducing calcium influx and CaN activation, which causes NFAT nuclear accumulation, leading to a pathological triad of dendritic spine loss, dendritic simplification, and neuritic dystrophies. Importantly, presented herein shows reversal of morphological neurodegenerative phenotypes in vitro and in vivo with a neuroprotective strategy of CaN inhibition, providing an important proof in principle of a non-Aβ-directed therapeutic intervention that improves neuronal structure in an AD model.

Memory loss in AD patients is correlated strongly with synaptic dysfunction (DeKosky and Scheff, 1990; Terry et al., 1991; Sze et al., 1997). CaN is a protein phosphatase that plays a fundamental role in memory formation through mechanisms controlling synaptic function (Malleret et al., 2001; Winder and Sweatt, 2001; Mansuy, 2003). Mice with inducible, hippocampal-restricted overexpression of CaNCA exhibit pronounced spatial learning and memory deficits in the Morris water maze task (Mansuy, 2003). The defect of learning behaviors is reversible in transgenic mice expressing a CaN inhibitory domain or application of antisense oligonucleotides (Malleret et al., 2001; Mansuy, 2003). These results suggest that synaptic dysfunction and memory retention deficits that occur in AD patients and animal AD models may result, at least in part, from altered CaN activity. Indeed, CaN inhibition with FK506 has been reported to improve memory function in APP transgenic mice (Dineley et al., 2007; Taglialatela et al., 2009).

As demonstrated herein, neurons from Tg cultures exhibit increased NFATc4 nuclear translocation (FIG. 5A), which can be regulated by CaN activation. The NFATc4-aberrant nuclear localization can also be induced by TgCM, which contains high levels of naturally secreted Aβ, and this can be blocked by immunodepletion of the TgCM with an Aβ-specific antibody, by the potent CaN inhibitory peptide AKAP79, or by treatment with VIVIT (SEQ ID NO: 7), which blocks NFAT activation (FIGS. 6C and 9A). Importantly, the NFATc4-aberrant nuclear localization and a constitutively active form of CaN were also detected in AD postmortem brain, indicating that CaN activation and the resulting downstream NFAT transcriptional cascade can also occur in AD. Moreover, in cultures, CaN inhibition or blockade of NFAT activation using VIVIT (SEQ ID NO: 7) provided significant neuroprotection from TgCM or APP overexpression-induced morphological deficits (FIGS. 8A to 8I; FIGS. 9A to 9I).

CaN activation has profound effects on neuronal morphology. As demonstrated in both cultures and wild-type adult mouse brain in vivo, manipulation of CaN activity by ectopic expression of CaNCA is sufficient to cause segmental spine loss, dendritic simplification, and focal swelling similar to Aβ-induced morphological aberrations (FIGS. 10A to 10H; FIG. 11). In mouse models of AD, CaN inhibition potently reduces the Aβ-related morphological neurodegenerative changes that occur near plaques (FIGS. 12A to 12D). These data indicate a pivotal novel role of CaN activation and CaN-mediated NFAT signaling pathway in Aβ-related morphological neurodegeneration, providing a mechanistic link between calcium overload, activation of CaN, and Aβ-induced neuronal morphological destruction of neural systems.

There are three major implications of these studies. First, the findings presented herein confirm that Aβ induces CaN activation in vitro and in AD (Liu et al., 2005; Snyder et al., 2005; Hsieh et al., 2006; Agostinho et al., 2008; Reese et al., 2008; Abdul et al., 2009); and demonstrate that CaN activation in neurons causes the pathological triad of dendritic spine loss, dendritic simplification, and dystrophies. Although it seems that activation of CaN has multiple effects, including functions at the dendritic spine itself (Hsieh et al., 2006), and it remains possible that VIVIT (SEQ ID NO: 7) disrupts CaN functions other than NFAT dephosphorylation (Oliveria et al., 2007), the data presented herein are consistent with the possibility that NFAT-mediated cascades play a prominent role in the neurodegenerative process. These results indicate a soluble form of Aβ, likely oligomeric, as the responsible bioactive molecule present in the vicinity of plaques that mediates the neuronal alterations that occur within a halo near plaques (Koffie et al., 2009). Importantly, these results allow the development of a simple in vitro model of Aβ-induced neurodegeneration (TgCM treatment of wild-type neurons for 24 h) that has predictive value for plaque-induced neurodegeneration in 8- to 12-month-old transgenic animals.

Second, activated CaN produces a phenocopy of these Aβ effects. Importantly, immunodepletion of Aβ, inhibition of CaN, or blockade of NFAT alone can inhibit these changes and lead to recovery of neuronal structure. Previous studies have shown some recovery of neuronal lesions after treatment with antibodies directed against Aβ (Lombardo et al., 2003; Brendza et al., 2005; Spires-Jones et al., 2008). Because the data herein show that CaN activation is downstream of soluble Aβ neurotoxic effects, a strategy aimed at preventing or restoring CaN-mediated neural system damage, combined with approaches that reduce Aβ generation or promote its clearance, can be more effective than either strategy alone. The higher enrichment of activated CaN and NFATc4 in the nuclear fraction of cortex from AD patients (FIGS. 5C to 5G) reinforces the importance and disease relevance of these observations. Thus, CaN inhibition or blockade of NFAT or its targets can be an important avenue for consideration in AD therapeutics.

Third, the results demonstrated herein indicate for the first time that CaN and NFAT play a major role in sculpting neural systems in the mature brain, indicating that the adult brain is not “hardwired.” A prominent role for CaN has been long established in learning and memory (Malleret et al., 2001; Winder and Sweatt, 2001; Zeng et al., 2001; Mansuy, 2003), and CaN activation is important in phenomena such as long-term depression. A recent study suggests that CaN-NFAT signaling is responsible for dendritic simplification during developmental pruning of the Xenopus neural system (Schwartz et al., 2009). However, a major role for CaN-NFAT-mediated transcriptional events in dendrite remodeling in the adult brain or in disease conditions, e.g., AD, has not been reported previously. Because aberrant CaN activation has been implicated in CNS trauma, ischemia, kainate injury, and glaucoma (Morioka et al., 1999; Springer et al., 2000; Wu et al., 2004; Huang et al., 2005; Uchino et al., 2008), each of which leads to neural system degeneration, it is envisioned that the type of structural remodeling and morphological degenerative changes observed in multiple conditions can be a consequence of CaN activation. Intriguingly, the major effects of CaN in AD can be mediated NFAT, indicating a potential unexplored role for transcriptional cascades in these conditions. Thus, a molecular mechanism of neurodegeneration in AD can be Aβ-induced aberrant activation of potent NFAT-mediated developmental program of neural system remodeling, indicating possible therapeutic avenues for rescue.

### REFERENCES

Abdul H M, Sama M A, Furman J L, Mathis D M, Beckett T L, Weidner A M, Patel E S, Baig I, Murphy M P, LeVine H 3rd, Kraner S D, Norris C M (2009) Cognitive decline in Alzheimer's disease is associated with selective changes in calcineurin/NFAT signaling. J Neurosci 29:12957-12969.
Agostinho P, Lopes J P, Velez Z, Oliveira C R (2008) Overactivation of calcineurin induced by amyloid-beta and prion proteins. Neurochem Int 52:1226-1233.
Aramburu J, Yaffe M B, López-Rodríguez C, Cantley L C, Hogan P G, Rao A (1999) Affinity-driven peptide selection of an NFAT inhibitor more selective than cyclosporin A. Science 285:2129-2133.
Bacskai B J, Hickey G A, Skoch J, Kajdasz S T, Wang Y, Huang G F, Mathis C A, Klunk W E, Hyman B T (2003) Four-dimensional multiphoton imaging of brain entry, amyloid binding, and clearance of an amyloid-beta ligand in transgenic mice. Proc Natl Acad Sci USA 100:12462-12467.
Berridge M J, Lipp P, Bootman M D (2000) The versatility and universality of calcium signalling. Nat Rev Mol Cell Biol 1:11-21.
Bezprozvanny I, Mattson M P (2008) Neuronal calcium mishandling and the pathogenesis of Alzheimer's disease. Trends Neurosci 31:454-463.
Brendza R P, Bacskai B J, Cirrito J R, Simmons K A, Skoch J M, Klunk W E, Mathis C A, Bales K R, Paul S M, Hyman B T, Holtzman D M (2005) Anti-Abeta antibody treatment promotes the rapid recovery of amyloidassociated neuritic dystrophy in PDAPP transgenic mice. J Clin Invest 115:428-433.
Burkard N, Becher J, Heindl C, Neyses L, Schuh K, Ritter O (2005) Targeted proteolysis sustains calcineurin activation. Circulation 111:1045-1053.
Busche M A, Eichhoff G, Adelsberger H, Abramowski D, Wiederhold K H, Haass C, Staufenbiel M, Konnerth A, Garaschuk O (2008) Clusters of hyperactive neurons near amyloid plaques in a mouse model of Alzheimer's disease. Science 321:1686-1689.
Celsi F, Svedberg M, Unger C, Cotman C W, Carrì M T, Ottersen O P, Nordberg A, Torp R (2007) Beta-amyloid causes downregulation of calcineurin in neurons through induction of oxidative stress. Neurobiol Dis 26:342-352.
-Coghlan V M, Perrino B A, Howard M, Langeberg L K, Hicks J B, Gallatin W M, Scott J D (1995) Association of protein kinase A and protein phosphatase 2B with a common anchoring protein. Science 267:108-111.
DeKosky S T, Scheff S W (1990) Synapse loss in frontal cortex biopsies in Alzheimer's disease: correlation with cognitive severity. Ann Neurol 27:457-464.
Dineley K T, Hogan D, Zhang W R, Taglialatela G (2007) Acute inhibition of calcineurin restores associative learning and memory in Tg2576 APP transgenic mice. Neurobiol Learn Mem 88:217-224.
Geula C, Wu C K, Saroff D, Lorenzo A, Yuan M, Yankner B A (1998) Aging renders the brain vulnerable to amyloid beta-protein neurotoxicity. Nat Med 4:827-831.
Graef I A, Wang F, Charron F, Chen L, Neilson J, Tessier-Lavigne M, Crabtree G R (2003) Neurotrophins and netrins require calcineurin/NFAT signaling to stimulate outgrowth of embryonic axons. Cell 113:657-670.
Groth R D, Mermelstein P G (2003) Brain-derived neurotrophic factor activation of NFAT (nuclear factor of activated T-cells)-dependent transcription: a role for the transcription factor NFATc4 in neurotrophin-mediated gene expression. J Neurosci 23:8125-8134.
Grynkiewicz G, Poenie M, Tsien R Y (1985) A new generation of Ca²⁺ indicators with greatly improved flourescence properties. J Biol Chem 260: 3440-3450.
Halpain S, Hipolito A, Saffer L (1998) Regulation of F-actin stability in dendritic spines by glutamate receptors and calcineurin. J Neurosci 18:9835-9844.
Hardy J, Allsop D (1991) Amyloid deposition as the central event in the aetiology of Alzheimer's disease. Trends Pharmacol Sci 12:383-388.
Hardy J, Selkoe D J (2002) The amyloid hypothesis of Alzheimer's disease: progress and problems on the road to therapeutics. Science 297:353-356.
Hsiao K, Chapman P, Nilsen S, Eckman C, Harigaya Y, Younkin S, Yang F, Cole G (1996) Correlative memory deficits, Abeta elevation, and amyloid plaques in transgenic mice. Science 274:99-102.
Hsieh H, Boehm J, Sato C, Iwatsubo T, Tomita T, Sisodia S, Malinow R (2006) AMPAR removal underlies Abeta-induced synaptic depression and dendritic spine loss. Neuron 52:831-843.
Huang W, Fileta J B, Dobberfuhl A, Filippopolous T, Guo Y, Kwon G, Grosskreutz C L (2005) Calcineurin cleavage is triggered by elevated intraocular pressure, and calcineurin inhibition blocks retinal ganglion cell death in experimental glaucoma. Proc Natl Acad Sci USA 102: 12242-12247.
Johnson-Wood K, Lee M, Motter R, Hu K, Gordon G, Barbour R, Khan K, Gordon M, Tan H, Games D, Lieberburg I, Schenk D, Seubert P, McConlogue L (1997) Amyloid precursor protein processing and A beta42 deposition in a transgenic mouse model of Alzheimer disease. Proc Natl Acad Sci USA 94:1550-1555.
Klee C B, Crouch T H, Krinks M H (1979) Calcineurin: a calcium- and calmodulin-binding protein of the nervous system. Proc Natl Acad Sci USA 76:6270-6273.
Klunk W E, Bacskai B J, Mathis C A, Kajdasz S T, McLellan M E, Frosch M P, Debnath M L, Holt D P, Wang Y, Hyman B T (2002) Imaging Abeta plaques in living transgenic mice with multiphoton microscopy and methoxy-X04, a systemically administered Congo red derivative. J Neuropathol Exp Neurol 61:797-805.
Knowles R B, Wyart C, Buldyrev S V, Cruz L, Urbanc B, Hasselmo M E, Stanley H E, Hyman B T (1999) Plaque-induced neurite abnormalities: implications for disruption of neural networks in Alzheimer's disease. Proc Natl Acad Sci USA 96:5274-5279.
Koffie R M, Meyer-Luehmann M, Hashimoto T, Adams K W, Mielke M L, Garcia-Alloza M, Micheva K D, Smith S J, Kim M L, Lee V M, Hyman B T, Spires-Jones T L (2009) Oligomeric amyloid beta associates with postsynaptic densities and correlates with excitatory synapse loss near senile plaques. Proc Natl Acad Sci USA 106:4012-4017.
Kuchibhotla K V, Goldman S T, Lattarulo C R, Wu H Y, Hyman B T, Bacskai B J (2008) Abeta plaques lead to aberrant regulation of calcium homeostasis in vivo resulting in structural and functional disruption of neuronal networks. Neuron 59:214-225.
Le R, Cruz L, Urbanc B, Knowles R B, Hsiao-Ashe K, Duff K, Irizarry M C, Stanley H E, Hyman B T (2001) Plaque-induced abnormalities in neurite geometry in transgenic models of Alzheimer disease: implications for neural system disruption. J Neuropathol Exp Neurol 60:753-758.
Liu F, Grundke-Iqbal I, Iqbal K, Oda Y, Tomizawa K, Gong C X (2005) Truncation and activation of calcineurin A by calpain I in Alzheimer disease brain. J Biol Chem 280:37755-37762.
Lombardo J A, Stern E A, McLellan M E, Kajdasz S T, Hickey G A, Bacskai B J, Hyman B T (2003) Amyloid-β antibody treatment leads to rapid normalization of plaque-induced neuritic alterations. J Neurosci 23:10879-10883.
Lorenzo A, Yankner B A (1994) Beta-amyloid neurotoxicity requires fibril formation and is inhibited by congo red. Proc Natl Acad Sci USA 91:12243-12247.
Malleret G, Haditsch U, Genoux D, Jones M W, Bliss T V, Vanhoose A M, Weitlauf C, Kandel E R, Winder D G, Mansuy I M (2001) Inducible and reversible enhancement of learning, memory, and long-term potentiation by genetic inhibition of calcineurin. Cell 104:675-686.
Mansuy E M (2003) Calcineurin in memory and bidirectional plasticity. Biochem Biophys Res Commun 311:1195-1208.
Mattson M P (2004) Pathways towards and away from Alzheimer's disease. Nature 430:631-639.
Meyer-Luehmann M, Spires-Jones T L, Prada C, Garcia-Alloza M, de Calignon A, Rozkalne A, Koenigsknecht-Talboo J, Holtzman D M, Bacskai B J, Hyman B T (2008) Rapid appearance and local toxicity of amyloid-beta plaques in a mouse model of Alzheimer's disease. Nature 451:720-724.
Morioka M, Hamada J, Ushio Y, Miyamoto E (1999) Potential role of calcineurin for brain ischemia and traumatic injury. Prog Neurobiol 58:1-30.
Mulkey R M, Endo S, Shenolikar S, Malenka R C (1994) Involvement of a calcineurin/inhibitor-1 phosphatase cascade in hippocampal long-term depression. Nature 369:486-488.
Oliveria S F, Dell'Acqua M L, Sather W A (2007) AKAP79/150 anchoring of calcineurin controls neuronal L-type Ca²⁺ channel activity and nuclear signaling. Neuron 55:261-275.
Palotás A, Kálmán J, Palotás M, Juhász A, Janka Z, Penke B (2002) Fibroblasts and lymphocytes from Alzheimer patients are resistant to beta-amyloid-induced increase in the intracellular calcium concentration. Prog Neuropsychopharmacol Biol Psychiatry 26:971-974.
Pike C J, Burdick D, Walencewicz A J, Glabe C G, Cotman C W (1993) Neurodegeneration induced by β-amyloid peptides in vitro: the role of peptide assembly state. J Neurosci 13:1676-1687.
Reese L C, Zhang W, Dineley K T, Kayed R, Taglialatela G (2008) Selective induction of calcineurin activity and signaling by oligomeric amyloid beta. Aging Cell 7:824-835.
Schwartz N, Schohl A, Ruthazer E S (2009) Neural activity regulates synaptic properties and dendritic structure in vivo through calcineurin/NFAT signaling. Neuron 62:655-669.
Shankar G M, Bloodgood B L, Townsend M, Walsh D M, Selkoe D J, Sabatini B L (2007) Natural oligomers of the Alzheimer amyloid-┌ protein induce reversible synapse loss by modulating an NMDA-type glutamate receptor-dependent signaling pathway. J Neurosci 27:2866-2875.
Shankar G M, Li S, Mehta T H, Garcia-Munoz A, Shepardson N E, Smith I, Brett F M, Farrell M A, Rowan M J, Lemere C A, Regan C M, Walsh D M, Sabatini B L, Selkoe D J (2008) Amyloid-beta protein dimers isolated directly from Alzheimer's brains impair synaptic plasticity and memory. Nat Med 14:837-842.
Smith I F, Green K N, LaFerla F M (2005) Calcium dysregulation in Alzheimer's disease: recent advances gained from genetically modified animals. Cell Calcium 38:427-437.
Snyder E M, Nong Y, Almeida C G, Paul S, Moran T, Choi E Y, Nairn A C, Salter M W, Lombroso P J, Gouras G K, Greengard P (2005) Regulation of NMDA receptor trafficking by amyloid-beta. Nat Neurosci 8:1051-1058.
Spires-Jones T L, de Calignon A, Matsui T, Zehr C, Pitstick R, Wu H Y, Osetek J D, Jones P B, Bacskai B J, Feany M B, Carlson G A, Ashe K H, Lewis J, Hyman B T (2008) In vivo imaging reveals dissociation between caspase activation and acute neuronal death in tangle-bearing neurons. J Neurosci 28:862-867.
Spires T L, Meyer-Luehmann M, Stern E A, McLean P J, Skoch J, Nguyen P T, Bacskai B J, Hyman B T (2005) Dendritic spine abnormalities in amyloid precursor protein transgenic mice demonstrated by gene transfer and intravital multiphoton microscopy. J Neurosci 25:7278-7287.
Springer J E, Azbill R D, Nottingham S A, Kennedy S E (2000) Calcineurinmediated BAD dephosphorylation activates the caspase-3 apoptotic cascade in traumatic spinal cord injury. J Neurosci 20:7246-7251.
Stutzmann G E (2005) Calcium dysregulation, IP3 signaling, and Alzheimer's disease. Neuroscientist 11:110-115.
Sze C I, Troncoso J C, Kawas C, Mouton P, Price D L, Martin L J (1997) Loss of the presynaptic vesicle protein synaptophysin in hippocampus correlates with cognitive decline in Alzheimer disease. J Neuropathol Exp Neurol 56:933-944.
Taglialatela G, Hogan D, Zhang W R, Dineley K T (2009) Intermediate- and long-term recognition memory deficits in Tg2576 mice are reversed with acute calcineurin inhibition. Behav Brain Res 200:95-99.
Terry R D, Masliah E, Salmon D P, Butters N, DeTeresa R, Hill R, Hansen L A, Katzman R (1991) Physical basis of cognitive alterations in Alzheimer's disease: synapse loss is the major correlate of cognitive impairment. Ann Neurol 30:572-580.
Townsend M, Shankar G M, Mehta T, Walsh D M, Selkoe D J (2006) Effects of secreted oligomers of amyloid beta-protein on hippocampal synaptic plasticity: a potent role for trimers. J Physiol 572:477-492.
Uchino H, Kuroda Y, Morota S, Hirabayashi G, Ishii N, Shibasaki F, Ikeda Y, Hansson M J, Elmér E (2008) Probing the molecular mechanisms of neuronal degeneration: importance of mitochondrial dysfunction and calcineurin activation. J Anesth 22:253-262.
Walsh D M, Selkoe D J (2004) Deciphering the molecular basis of memory failure in Alzheimer's disease. Neuron 44:181-193.
Wang J H, Kelly P T (1996) The balance between postsynaptic Ca²⁺-dependent protein kinase and phosphatase activities controlling synaptic strength. Learn Mem 3:170-181.
Winder D G, Sweatt J D (2001) Roles of serine/threonine phosphatases in hippocampal synaptic plasticity. Nat Rev Neurosci 2:461-474.
Wu H Y, Tomizawa K, Oda Y, Wei F Y, Lu Y F, Matsushita M, Li S T, Moriwaki A, Matsui H (2004) Critical role of calpain-mediated cleavage of calcineurin in excitotoxic neurodegeneration. J Biol Chem 279:4929-4940.
Yaari R, Corey-Bloom J (2007) Alzheimer's disease. Semin Neurol 27: 32-41.
Zeng H, Chattarji S, Barbarosie M, Rondi-Reig L, Philpot B D, Miyakawa T, Bear M F, Tonegawa S (2001) Forebrain-specific calcineurin knockout selectively impairs bidirectional synaptic plasticity and working/episodic like memory. Cell 107:617-629.

SEQUENCES
(SEQ ID NO: 1)
VIVIT
(SEQ ID NO: 2)
5′-GAATTCATGTATCCGTATGACGTACCAGAGTACGCCATGTCCGAGCCCAAGGCGATTGATCC
(SEQ ID NO: 3)
3′GCTAGCTCACGTACTGTCGAGTCCCAGGAGAGGGTTTGGGATCGGCTTGCCCTGGATATTGCTGCTATTACTG
CCATTGC
(SEQ ID NO: 4)
3′-CTAGTTCTGATGACTTCCTTCCGGGCTGCGGCCGTC
(SEQ ID NO: 5)
5′-GAAGTTATCAGTCGACATGGACTACAAAGACGATGACGACA-AGGGCAGGAAGTGTCCACAA
(SEQ ID NO: 6)
3′-ATG-GTCTAGAAAGCTTCTAGACATTTTTAGATTTTGTAACATCAAATTCACTGATTTC.

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating Alzheimer's disease (AD) in a subject in need thereof, comprising contacting a population of neuronal cells in the subject with an effective amount of a nuclear factor of activated T cells (NFAT) antagonist.

2. The method of claim 1, wherein the population of neuronal cells is in proximity to an amyloid-beta deposit.

3. (canceled)

4. (canceled)

5. (canceled)

6. The method of claim 1, wherein the effective amount is sufficient to increase dendritic spine density of one or more neuronal cells by at least about 5%, as compared to neuronal cells in the absence of the NFAT antagonist.

7. (canceled)

8. (canceled)

9. The method of claim 1, wherein the NFAT antagonist is selected from the group consisting of a small molecule, a nucleic acid, a protein, a peptide, and an intrabody.

10. The method of claim 1, wherein the NFAT antagonist is a peptide comprising an amino acid sequence of VIVIT (SEQ ID NO: 7).

11. (canceled)

12. (canceled)

13. (canceled)

14. The method of claim 1, further comprising a step of diagnosing a subject with AD prior to the contacting.

15. The method of claim 1, wherein the subject is a human.

16. (canceled)

17. A method of inhibiting neurodengeneration, comprising contacting a population of neuronal cells with an effective amount of a nuclear factor of activated T cells (NFAT) antagonist.

18. (canceled)

19. (canceled)

20. The method of claim 17, wherein the effective amount is sufficient to increase dendritic spine density of one or more neuronal cells by at least about 5%, as compared to neuronal cells in the absence of the NFAT antagonist.

21. (canceled)

22. (canceled)

23. The method of claim 17, wherein the NFAT antagonist is selected from the group consisting of a small molecule, a nucleic acid, a protein, a peptide and an intrabody.

24. The method of claim 17, wherein the NFAT antagonist is a peptide comprising an amino acid sequence of VIVIT (SEQ ID NO: 7).

25. (canceled)

26. (canceled)

27. (canceled)

28. The method of claim 17, wherein the contacting is in vitro.

29. The method of claim 17, wherein the contacting is ex vivo.

30. The method of claim 17, wherein the contacting is in vivo.

31. (canceled)

32. The method of claim 30, wherein the in vivo contacting is in a subject diagnosed with or predisposed to a neurodegenerative disorder.

33. (canceled)

34. The method of claim 30, wherein the in vivo contacting is in a subject suffering from encephalitis or brain trauma.

35. The method of claim 30, wherein the in vivo contacting is in a subject suffering from tau-mediated synaptic degeneration.

36. The method of claim 30, wherein the in vivo contacting is in a subject suffering from frontotemporal dementia.

37. (canceled)

38. The method of claim 32, wherein the subject is a human.

39. The method claim 17, wherein the population of neuronal cells is in proximity to an amyloid-beta deposit.
